(19) United States
(12) Patent Application Publication
(10) Pub. No.: US 2010/0257778 A1
(43) Pub. Date: Oct. 14, 2010

(54) PRODUCTION OF FATTY ACID DERIVATIVES

(75) Inventors: Alfred Gaertner, South San Francisco, CA (US); Andreas Schirmer, South San Francisco, CA (US); Fernando Valle, Burlingame, CA (US); Stephen Del Cardayre, South San Francisco, CA (US)

Correspondence Address: LS9, Inc., 600 Gateway Boulevard, South San Francisco, CA, 94080, US

(73) Assignee: LS9, Inc., South San Francisco, CA (US)

(21) Appl. No.: 12/758,001

(22) Filed: Apr. 11, 2010

Related U.S. Application Data
(60) Provisional application No. 61/168,293, filed on Apr. 10, 2009.
(60) Provisional application No. 61/226,749, filed on Jul. 20, 2009.
(60) Provisional application No. 61/227,025, filed on Jul. 20, 2009.
(60) Provisional application No. 61/262,544, filed on Nov. 19, 2009.

(51) Int. Cl. C10L 1/19; C12P 7/62; C07C 69/003; C12P 7/64; C12N 1/00; C12N 1/21; C07C 53/00

(52) U.S. Cl. 44/388; 435/135; 560/129; 435/134; 435/243; 435/252.3; 435/252.35; 435/252.34; 435/252.33; 554/1

(57) ABSTRACT

Methods and compositions for producing fatty acid derivatives, for example, fatty esters, are described.

### CROSS REFERENCE TO RELATED APPLICATIONS

This application claims the benefit of U.S. Provisional Application Nos. 61/168,293, filed Apr. 10, 2009, 61/266,749, filed Jul. 20, 2009, 61/227,025, filed Jul. 20, 2009, and 61/262,544, filed Nov. 19, 2009, the entire content of each is hereby incorporated by reference.

### BACKGROUND OF THE INVENTION

Petroleum is a limited, natural resource found in the Earth in liquid, gaseous, or solid forms. Petroleum is primarily composed of hydrocarbons, which are comprised mainly of carbon and hydrogen. It also contains significant amounts of other elements, such as, nitrogen, oxygen, or sulfur, in different forms.

Petroleum is a valuable resource, but petroleum products are developed at considerable costs, both financial and environmental. First, sources of petroleum must be discovered. Petroleum exploration is an expensive and risky venture. The cost of exploring deep water wells can exceed $100 million. In addition to the economic cost, petroleum exploration carries a high environmental cost. For example, offshore exploration disturbs the surrounding marine environments.

After a productive well is discovered, the petroleum must be extracted from the Earth at great expense. Even under the best circumstances, only 50% of the petroleum in a well can be extracted. Petroleum extraction also carries an environmental cost. For example, petroleum extraction can result in large seepages of petroleum rising to the surface. Offshore drilling involves dredging the seabed which disrupts or destroys the surrounding marine environment.

After extraction, petroleum must be transported over great distances from petroleum producing regions to petroleum consuming regions. In addition to the shipping costs, there is also the environmental risk of devastating oil spills.

In its natural form, crude petroleum extracted from the Earth has few commercial uses. It is a mixture of hydrocarbons (e.g., paraffins (or alkanes), olefins (or alkenes), alkynes, napthenes (or cylcoalkanes), aliphatic compounds, aromatic compounds, etc.) of varying length and complexity. In addition, crude petroleum contains other organic compounds (e.g., organic compounds containing nitrogen, oxygen, sulfur, etc.) and impurities (e.g., sulfur, salt, acid, metals, etc.).

Hence, crude petroleum must be refined and purified before it can be used commercially. Due to its high energy density and its easy transportability, most petroleum is refined into fuels, such as transportation fuels (e.g., gasoline, diesel, aviation fuel, etc.), heating oil, liquefied petroleum gas, etc.

Crude petroleum is also a primary source of raw materials for producing petrochemicals. The two main classes of raw materials derived from petroleum are short chain olefins (e.g., ethylene and propylene) and aromatics (e.g., benzene and xylene isomers). These raw materials are derived from the longer chain hydrocarbons in crude petroleum by cracking the long chain hydrocarbons at considerable expense using a variety of methods, such as catalytic cracking, steam cracking, or catalytic reforming. These raw materials are used to make petrochemicals, which cannot be directly refined from crude petroleum, such as monomers, solvents, detergents, or adhesives.

One example of a raw material derived from crude petroleum is ethylene. Ethylene is used to produce petrochemicals such as, polyethylene, ethanol, ethylene oxide, ethylene glycol, polyester, glycol ether, ethoxylate, vinyl acetate, 1,2-dichloroethane, trichloroethylene, tetrachloroethylene, vinyl chloride, and polyvinyl chloride. Another example of a raw material derived from crude petroleum is propylene. Propylene is used to produce isopropyl alcohol, acrylonitrile, polypropylene, propylene oxide, propylene glycol, glycol ethers, butylene, isobutylene, 1,3-butadiene, synthetic elastomers, polyolefins, alpha-olefins, fatty alcohols, acrylic acid, acrylic polymers, allyl chloride, epichlorohydrin, and epoxy resins.

Petrochemicals can be used to make specialty chemicals, such as plastics, resins, fibers, elastomers, pharmaceuticals, lubricants, or gels. Examples of specialty chemicals which can be produced from petrochemical raw materials are: fatty acids, hydrocarbons (e.g., long chain hydrocarbons, branched chain hydrocarbons, saturated hydrocarbons, unsaturated hydrocarbons, etc.), fatty alcohols, esters, fatty aldehydes, ketones, lubricants, etc.

Specialty chemicals have many commercial uses. Fatty acids are used commercially as surfactants. Surfactants can be found in detergents and soaps. Fatty acids can also be used as additives in fuels, lubricating oils, paints, lacquers, candles, salad oils, shortenings, cosmetics, and emulsifiers. In addition, fatty acids are used as accelerator activators in rubber products. Fatty acids can also be used as a feedstock to produce methyl esters, amides, amines, acid chlorides, anhydrides, ketene dimers, and peroxy acids and esters.

Esters have many commercial uses. For example, biodiesel, an alternative fuel, is comprised of esters (e.g., fatty acid methyl ester, fatty acid ethyl esters, etc.). Some low molecular weight esters are volatile with a pleasant odor which makes them useful as fragrances or flavoring agents. In addition, esters are used as solvents for lacquers, paints, and varnishes. Furthermore, some naturally occurring substances, such as waxes, fats, and oils are comprised of esters. Esters are also used as softening agents in resins and plastics, plasticizers, flame retardants, and additives in gasoline and oil. In addition, esters can be used in the manufacture of polymers, films, textiles, dyes, and pharmaceuticals.

In addition, crude petroleum is a source of lubricants. Lubricants derived petroleum are typically composed of olefins, particularly polyolefins and alpha-olefins. Lubricants can either be refined from crude petroleum or manufactured using the raw materials refined from crude petroleum.

Obtaining these specialty chemicals from crude petroleum requires a significant financial investment as well as a great deal of energy. It is also an inefficient process because frequently the long chain hydrocarbons in crude petroleum are cracked to produce smaller monomers. These monomers are then used as the raw material to manufacture the more complex specialty chemicals.

In addition to the problems with exploring, extracting, transporting, and refining petroleum, petroleum is a limited and dwindling resource. One estimate of current world petroleum consumption is 30 billion barrels per year. By some estimates, it is predicted that at current production levels, the world's petroleum reserves could be depleted before the year 2050.

Finally, the burning of petroleum based fuels releases greenhouse gases (e.g., carbon dioxide) and other forms of air pollution (e.g., carbon monoxide, sulfur dioxide, etc.). As the world's demand for fuel increases, the emission of greenhouse gases and other forms of air pollution also increases. The accumulation of greenhouse gases in the atmosphere leads to an increase in global warming. Hence, in addition to damaging the environment locally (e.g., oil spills, dredging of marine environments, etc.), burning petroleum also damages the environment globally.

Due to the inherent challenges posed by petroleum, there is a need for a renewable petroleum source which does not need to be explored, extracted, transported over long distances, or substantially refined like petroleum. There is also a need for a renewable petroleum source that can be produced economically. In addition, there is a need for a renewable petroleum source that does not create the type of environmental damage produced by the petroleum industry and the burning of petroleum based fuels. For similar reasons, there is also a need for a renewable source of chemicals that are typically derived from petroleum.

Renewable energy sources, such as sunlight, water, wind, and biomass, are a potential alternative to petroleum fuels. Biofuel is a biodegradable, clean-burning combustible fuel produced from biomass, and can be made of alkanes and esters. An exemplary biofuel is biodiesel. Biodiesel can be used in most internal combustion diesel engines in either a pure form, which is referred to as “neat” biodiesel, or as a mixture in any concentration with regular petroleum diesel.

Biodiesel offers advantages compared to petroleum-based diesel, including reduced emissions (e.g., carbon monoxide, sulphur, aromatic hydrocarbons, soot particles) during combustion. Biodiesel also maintains a balanced carbon dioxide cycle because it is based on renewable biological materials. Biodiesel is typically biodegradable, and imparts enhanced safety due to its high flash point and low flammability. Furthermore, biodiesel provides good lubrication properties, thereby reducing wear and tear on engines.

Current methods of making biodiesel involve transesterification of triacylglycerides from vegetable oil feedstocks, such as rapeseed in Europe, soybean in North America, and palm oil in South East Asia. Industrial-scale biodiesel production is thus geographically and seasonally restricted to areas where vegetable oil feedstocks are produced. The transesterification process leads to a mixture of fatty esters which can be used as biodiesel. However, glycerin is an undesirable byproduct of the transesterification process. To be usable as biodiesel, the fatty esters must be further purified from the heterogeneous product. This increases costs and the amount of energy required for fatty ester production and, ultimately, biodiesel production as well. Furthermore, vegetable oil feedstocks are inefficient sources of energy because they require extensive acreage for cultivation. For example, the yield of biodiesel from rapeseed is only 1300 L/hectare because only the seed oil is used for biodiesel production, while the rest of the rapeseed biomass is discarded. Additionally, cultivating some vegetable oil feedsocks, such as rapeseed and soybean, requires frequent crop rotation to prevent nutrient depletion of the land.

Thus there is a need for an economically- and energy-efficient biofuel, and methods of making biofuels from renewable energy sources such as biomass.

### SUMMARY OF THE INVENTION

The invention is based, at least in part, on the production of fatty esters, such as fatty esters, including, for example fatty acid methyl esters (“FAME”) and fatty acid ethyl esters (“FAEE”), from genetically engineered microorganisms. Accordingly, in one aspect, the invention features a method of producing a fatty ester. The method comprises culturing a host cell in the presence of a carbon source, wherein the host cell is genetically engineered to overexpress a gene encoding a thioesterase, a gene encoding an acyl-CoA synthase, and a gene encoding an ester synthase. In some embodiments, the method further comprises isolating the fatty ester.

In some embodiments, the fatty ester is present in the extracellular environment. In some embodiments, the fatty ester is isolated from the extracellular environment of the host cell. In some embodiments, the fatty ester is spontaneously secreted, partially or completely, from the host cell. In alternative embodiments, the fatty ester is transported into the extracellular environment, optionally with the aid of one or more suitable transport proteins. In other embodiments, the fatty ester is passively transported into the extracellular environment.

In some embodiments, the method further comprises culturing the host cell in the presence of an alcohol. In certain embodiments, the alcohol is methanol or ethanol. In some embodiments, the methanol or ethanol is present at a concentration of about 1 mL/L to about 100 mL/L. For example, the methanol or ethanol is present at a concentration of about 1 mL/L or more (e.g., about 1 mL/L or more, about 5 mL/L or more, about 10 mL/L or more). In alternative embodiments, the methanol or ethanol is present at a concentration of about 100 mL/L or less (e.g., about 100 mL/L or less, about 90 mL/L or less, about 80 mL/L or less).

In certain embodiments, the gene encoding a thioesterase may be, for example, tesA, 'tesA, tesB, fatB, fatB2, fatB3, fatA1, or fatA. In some embodiments, the gene encoding an acyl-CoA synthase may be, for example, fadD, fadK, BH3103, pfl-4354, EAV15023, fadD1, fadD2, RPC_{—}4074, fadDD35, fadDD22, faa39, the gene encoding the protein of GenBank Accession No. ZP_{—}01644857, or yhfL. In yet other embodiments, the gene encoding an ester synthase may be, for example, one encoding an enzyme of enzyme classification EC 2.3.1.75 or EC 2.3.1.20.

In other embodiments, the host cell is genetically engineered to express, relative to a wild type host cell, a decreased level of at least one of a gene encoding an acyl-CoA dehydrogenase, a gene encoding an outer membrane protein receptor, and a gene encoding a transcriptional regulator of fatty acid biosynthesis.

In some embodiments, the gene encoding an acyl-CoA dehydrogenase is fadE. In some embodiments, the gene encoding an outer membrane protein receptor encodes an outer membrane ferrichrome transporter, for example, fhuA. Yet in other embodiments, the gene encoding a transcriptional regulator of fatty acid biosynthesis encodes a DNA transcription repressor, for example, fabR.

In some embodiments, the host cell is cultured in a culture medium comprising an initial concentration of the carbon source of about 2 g/L to about 100 g/L. In other embodiments, the culture medium comprises an initial concentration of about 2 g/L to about 10 g/L of a carbon source, of about 10 g/L to about 20 g/L of a carbon source, of about 20 g/L to about 30 g/L of a carbon source, of about 30 g/L to about 40 g/L of a carbon source, or of about 40 g/L to about 50 g/L of a carbon source. In exemplary embodiments, the culture medium comprises an initial concentration of about 2 g/L or more (e.g., about 2 g/L or more, about 5 g/L or more, about 10 g/L or more) of a carbon source.

In some embodiments, the method further includes the step of monitoring the level of the carbon source in the culture medium. In some embodiments, the method further includes adding a supplemental carbon source to the culture medium when the level of the carbon source in the medium is less than about 0.5 g/L. In some embodiments, supplemental carbon source is added to the culture medium when the level of the carbon source in the medium is less than about 0.4 g/L, less than about 0.3 g/L, less than about 0.2 g/L, or less than about 0.1 g/L.

In some embodiments, the supplemental carbon source is added to maintain a carbon source level of about 2 g/L to about 5 g/L. In some embodiments, the supplemental carbon source is added to maintain a carbon source level of about 2 g/L or more (e.g., about 2 g/L or more, about 3 g/L or more, about 4 g/L or more). In certain embodiments, the supplemental carbon source is added to maintain a carbon source level of about 5 g/L or less (e.g., about 5 g/L or less, about 4 g/L or less, about 3 g/L or less). In some embodiments, the supplemental carbon source is added to maintain a carbon source level of about 2 g/L to about 3 g/L, of about 3 g/L to about 4 g/L, or of about 4 g/L to about 5 g/L. In some embodiments, the carbon source is glucose.

In some embodiments, the fatty acid methyl ester is produced at a concentration of about 1 g/L to about 200 g/L. In some embodiments, the fatty acid methyl ester is produced at a concentration of about 1 g/L or more (e.g., about 1 g/L or more, about 5 g/L or more, about 10 g/L or more, about 20 g/L or more, about 30 g/L or more). In some embodiments, the fatty acid methyl ester is produced at a concentration of about 1 g/L to about 170 g/L, of about 1 g/L to about 10 g/L, of about 40 g/L to about 170 g/L, of about 100 g/L to about 170 g/L, of about 10 g/L to about 100 g/L, of about 1 g/L to about 40 g/L, of about 40 g/L to about 100 g/L, or of about 1 g/L to about 100 g/L.

In some embodiments, the host cell is selected from the group consisting of a mammalian cell, plant cell, insect cell, yeast cell, fungus cell, filamentous fungi cell, and bacterial cell.

In particular embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In other embodiments, the host cell is a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell. In other embodiments, the host cell is an Actinomycetes cell. In other embodiments, the host cell is a Saccharomyces cerevisiae cell.

In yet other embodiments, the host cell is a cell from an eukaryotic plant, algae, cyanobacterium, green-sulfur bacterium, green non-sulfur bacterium, purple sulfur bacterium, purple non-sulfur bacterium, extremophile, yeast, fungus, engineered organisms thereof, or a synthetic organism. In some embodiments, the host cell is light dependent or fixes carbon. In some embodiments, the host cell has autotrophic activity. In some embodiments, the host cell has photoautotrophic activity, such as in the presence of light. In certain embodiments, the host cell is a cell from Arabidopsis thaliana, Panicum virgatums, Miscanthus giganteus, Zea mays, botryococcuse braunii, Chalamydomonas reinhardtii, Dunaliela salina, Thermosynechococcus elongatus, Chlorobium tepidum, Chloroflexus auranticus, Chromatiumm vinosum, Rhodospirillum rubrum, Rhodobacter capsulatus, Rhodopseudomonas palusris, Clostridium ljungdahlii, Clostridiuthermocellum, or Pencillium chrysogenum. In certain other embodiments, the host cell is from Pichia pastories, Saccharomyces cerevisae, Yarrowia lipolytica, Schizosaccharomyces pombe, Pseudomonas fluorescens, or Zymomonas mobilis. In yet further embodiments, the host cell is a cell from Synechococcus sp. PCC 7002, Synechococcus sp. PCC 7942, or Synechocystis sp. PCC6803.

In some embodiments, the host cell is a CHO cell, a COS cell, a VERO cell, a BHK cell, a HeLa cell, a Cv1 cell, an MDCK cell, a 293 cell, a 3T3 cell, or a PC12 cell.

In particular embodiments, the host cell is an E. coli cell. In some embodiments, the E. coli cell is a strain B, a strain C, a strain K, or a strain W E. coli cell.

In some embodiments, the invention pertains to a genetically engineered microorganism capable of producing fatty esters under conditions that allow product production. In some embodiments, the genetically engineered microorganism comprises one or more of at least one or more of at least one of a gene encoding a thioesterase, a gene encoding an acyl-CoA synthase, and a gene encoding an ester synthase. In certain embodiments, the genetically engineered microorganism comprises a gene encoding a thioesterase, a gene encoding an acyl-CoA synthase, and a gene encoding an ester synthase. In certain embodiments, two or more of the genes encoding a thioesterase, a gene encoding an acyl-CoA synthase, and a gene encoding an ester synthase are linked in a single operon. In certain other embodiments, all three of the genes encoding a thioesterase, a gene encoding an acyl-CoA synthase, and a gene encoding an ester synthase are linked into a single operon.

In certain embodiment, the genetically engineered microorganism comprises an exogenous control sequence stably incorporated into the genomic DNA of the microorganism upstream of one or more of at least one of a gene encoding a thioesterase, a gene encoding an acyl-CoA synthase, and a gene encoding an ester synthase. In an exemplary embodiment, the microorganism is engineered such that it comprises an exogenous control sequence stably incorporated into the genomic DNA of the microorganism up stream of a gene encoding a thioesterase, an acyl-CoA synthase and a gene encoding an ester synthase. In certain embodiments, the microorganism engineered as such produces an increased level of a fatty ester relative to a wild-type microorganism. In certain embodiments, the exogenous control sequence is, for example, a promoter. Exemplary promoters include, without limitation, a developmentally-regulated, organelle-specific, tissue-specific, inducible, constitutive, or cell-specific promoter.

In further embodiments, the microorganism is genetically engineered to express, relative to a wild type microorganism, a decreased level of at least one of a gene encoding an acyl-CoA dehydrogenase, a gene encoding an outer membrane protein receptor, and a gene encoding a transcriptional regulator of fatty acid biosynthesis. In certain embodiments, the microorganism is genetically engineered such that at least one of a gene encoding an acyl-CoA dehydrogenase, a gene encoding an outer membrane protein receptor, and a gene encoding a transcriptional regulator of fatty acid biosynthesis is deleted. In certain embodiments, the gene encoding the acyl-CoA dehydrogenase is fadE. In other embodiments, the gene encoding an outer membrane protein receptor encodes an outer membrane ferrichrome transporter, for example, fhuA. In further embodiments, the gene encoding a transcriptional regulator of fatty acid biosynthesis encodes a DNA transcription repressor, for example, fabR.

In certain embodiments, the genetically engineered microorganism can suitably be selected from a Gram-negative or a Gram-positive bacterium. In some embodiments, the genetically engineered microorganism is selected from an E. coli, mycrobacterium, Nocardia sp., Nocardia farcinica, Streptomyces griseus, Salinispora arenicola, Clavibacter michiganenesis, Acinetobacter, Alcanivorax, Alcaligenes, Arabidopsis, Fundibacter, Marinobacter, Mus musculus , Pseudomonas, or Simmodsia, Yarrowia, Candida, Rhodotorula, Rhodosporidium, Cryptococcus, Trichosporon, or Lipomyces. In certain embodiments, the genetically engineered microorganism is selected from an E. coli strain B, strain C, strain K or strain W. In further embodiments, the genetically engineered microorganism is selected from Synechococcus sp. PCC7002, Synechococcus elongatus PCC 7942, or Synechoystis sp. PCC 6803.

In some embodiments, the invention further features a method of producing a fatty ester comprising culturing the genetically engineered microorganism in the presence of a suitable alcohol substrate. In certain embodiments, the alcohol substrate can be selected from ethanol or methanol. In a preferred embodiment, the alcohol substrate is a methanol. In further embodiments, the method further includes culturing the genetically engineered microorganism under conditions that allows it to produce the fatty ester product.

In some embodiments, the fatty ester is produced at a yield of about 0.5 g to about 50 g of fatty ester per 100 g of glucose in the culture medium. For example, the fatty ester is produced at a yield of about 0.5 g of fatty ester per 100 g of glucose or more (e.g., about 0.5 g of fatty ester per 100 g of glucose or more, about 2 g of fatty ester per 100 g of glucose or more, about 5 g of fatty ester per 100 g of glucose or more, about 5 g of fatty ester per 100 g of glucose or more, about 10 g of fatty ester per 100 g of glucose or more). In particular embodiments, the fatty ester is produced at a yield of about 0.5 g to about 40 g of fatty ester per 100 g of glucose, about 0.5 g to about 30 g of fatty ester per 100 g of glucose, about 0.5 g to about 20 g of fatty ester per 100 g of glucose, about 0.5 g to about 10 g of fatty ester per 100 g of glucose, about 0.5 g to about 5 g of fatty ester per 100 g of glucose, or about 0.5 g to about 4 g of fatty ester per 100 g of glucose in the culture medium. In particular embodiments, the fatty ester is produced at a yield of at least 0.5 g of fatty ester, at least 4 g of fatty ester, at least 5 g of fatty ester, at least 10 g of fatty ester, at least 20 g of fatty ester, at least 30 g of fatty ester, at least 40 g of fatty ester, or at least 50 g of fatty ester per 100 g of glucose in the culture medium. In particular embodiments, the fatty ester is produced at a yield of no more than 50 g of fatty ester per 100 g of glucose in the culture medium.

In some embodiments, the fatty ester is produced at a yield of about 0.5% to about 50% by mass of the glucose in the culture medium. For example, the fatty ester is produced at a yield of about 0.5% or more (e.g., about 0.5% or more, about 2% or more, about 5% or more) by mass of the glucose in the culture medium. In particular embodiments, the fatty ester is produced at a yield of about 0.5% to about 40%, about 0.5% to about 30%, about 0.5% to about 20%, about 0.5% to about 10%, about 0.5% to about 5%, or about 0.5% to about 4% by mass of the glucose in the culture medium. In particular embodiments, the fatty ester is produced at a yield of at least about 0.5%, at least about 4%, at least about 5%, at least about 10%, at least about 20%, at least about 30%, at least about 40%, or at least about 50% by mass of glucose in the culture medium. In particular embodiments, the fatty ester is produced at a yield of no more than 50% by mass of glucose in the culture medium.

In some embodiments, the fatty ester is produced at a yield of about 10% to about 95% by mass of carbon in the carbon source in the culture medium. In particular embodiments, the fatty ester is produced at a yield of about 15% to about 90%, about 20% to about 80%, or about 30% to about 70% by mass of carbon in the carbon source in the culture medium. In particular embodiments, the fatty ester is produced at a yield of at least about 10%, at least about 20%, at least about 30%, at least about 40%, at least about 50%, at least about 60%, at least about 70%, at least about 80%, at least about 90%, or at least about 95% by mass of carbon in the carbon source in the culture medium. In particular embodiments, the fatty ester is produced at a yield of no more than 95% by mass of carbon in the carbon source in the culture medium.

In some embodiments, the fatty ester is a fatty acid ethyl ester and is produced at a yield of about 0.5 g to about 50 g of fatty acid ethyl ester per 100 g of glucose in the culture medium. For example, the fatty acid ethyl ester and is produced at a yield of about 0.5 g or more (e.g., about 0.5 g or more, about 2 g or more, about 5 g or more, about 10 g or more, about 15 g or more) per 100 g of glucose in the culture medium. In particular embodiments, the fatty acid ethyl ester is produced at a yield of about 0.5 g to about 40 g of fatty acid ethyl ester per 100 g of glucose, about 0.5 g to about 30 g of fatty acid ethyl ester per 100 g of glucose, about 0.5 g to about 20 g of fatty acid ethyl ester per 100 g of glucose, about 0.5 g to about 10 g of fatty acid ethyl ester per 100 g of glucose, about 0.5 g to about 5 g of fatty acid ethyl ester per 100 g of glucose, or about 0.5 g to about 4 g of fatty acid ethyl ester per 100 g of glucose in the culture medium. In particular embodiments, the fatty acid ethyl ester is produced at a yield of at least 0.5 g of fatty acid ethyl ester, at least 4 g of fatty acid ethyl ester, at least 5 g of fatty acid ethyl ester, at least 10 g of fatty acid ethyl ester, at least 20 g of fatty acid ethyl ester, at least 30 g of fatty acid ethyl ester, at least 40 g of fatty acid ethyl ester, or at least 50 g of fatty acid ethyl ester per 100 g of glucose in the culture medium. In particular embodiments, the fatty acid ethyl ester is produced at a yield of no more than 50 g of fatty acid ethyl ester per 100 g of glucose in the culture medium.

In some embodiments, the fatty acid ethyl ester is produced at a yield of about 0.5% to about 50% by mass of the glucose in the culture medium. For example, the fatty acid ethyl ester is produced at a yield of about 0.5% or more (e.g., of about 0.5% or more, of about 1% or more, of about 2% or more of about 5% or more of about 10% or more) by mass of the glucose in the culture medium. In particular embodiments, the fatty acid ethyl ester is produced at a yield of about 0.5% to about 40%, about 0.5% to about 30%, about 0.5% to about 20%, about 0.5% to about 10%, about 0.5% to about 5%, or about 0.5% to about 4% by mass of the glucose in the culture medium. In particular embodiments, the fatty acid ethyl ester is produced at a yield of at least about 0.5%, at least about 4%, at least about 5%, at least about 10%, at least about 20%, at least about 30%, at least about 40%, or at least about 50% by mass of glucose in the culture medium. In particular embodiments, the fatty acid ethyl ester is produced at a yield of no more than 50% by mass of glucose in the culture medium.

In some embodiments, the fatty acid ethyl ester is produced at a yield of about 10% to about 95% by mass of carbon in the carbon source in the culture medium. For example, the fatty acid ethyl ester is produced at a yield of about 10% or more (e.g., of about 10% or more, of about 15% or more, of about 20% or more, of about 25% or more) by pass of carbon in the carbon source in the culture medium. In particular embodiments, the fatty acid ethyl ester is produced at a yield of about 15% to about 90%, about 20% to about 80%, or about 30% to about 70% by mass of carbon in the carbon source in the culture medium. In particular embodiments, the fatty acid ethyl ester is produced at a yield of at least about 10%, at least about 20%, at least about 30%, at least about 40%, at least about 50%, at least about 60%, at least about 70%, at least about 80%, at least about 90%, or at least about 95% by mass of carbon in the carbon source in the culture medium. In particular embodiments, the fatty acid ethyl ester is produced at a yield of no more than 95% by mass of carbon in the carbon source in the culture medium.

In some embodiments, the fatty ester is a fatty acid methyl ester and is produced at a yield of about 0.5 g to about 50 g of fatty acid methyl ester per 100 g of glucose in the culture medium. For example, the fatty ester is a fatty acid methyl ester and is produced at a yield of about 0.5 g or more (e.g., about 0.5 g or more, about 1 g or more, about 2 g or more, about 5 g or more, about 10 g or more) of fatty acid methyl ester per 100 g of glucose in the culture medium. In particular embodiments, the fatty acid methyl ester is produced at a yield of about 0.5 g to about 40 g of fatty acid methyl ester per 100 g of glucose, about 0.5 g to about 30 g of fatty acid methyl ester per 100 g of glucose, about 0.5 g to about 20 g of fatty acid methyl ester per 100 g of glucose, about 0.5 g to about 10 g of fatty acid methyl ester per 100 g of glucose, about 0.5 g to about 5 g of fatty acid methyl ester per 100 g of glucose, or about 0.5 g to about 4 g of fatty acid methyl ester per 100 g of glucose in the culture medium. In particular embodiments, the fatty acid methyl ester is produced at a yield of at least 0.5 g of fatty acid methyl ester, at least 4 g of fatty acid methyl ester, at least 5 g of fatty acid methyl ester, at least 10 g of fatty acid methyl ester, at least 20 g of fatty acid methyl ester, at least 30 g of fatty acid methyl ester, at least 40 g of fatty acid methyl ester, or at least 50 g of fatty acid methyl ester per 100 g of glucose in the culture medium. In particular embodiments, the fatty acid methyl ester is produced at a yield of no more than 50 g of fatty acid methyl ester per 100 g of glucose in the culture medium.

In some embodiments, the fatty acid methyl ester is produced at a yield of about 0.5% to about 50% by mass of the glucose in the culture medium. For example, fatty acid methyl ester is produced at a yield of about 0.5% or more (e.g., about 0.5% or more, about 1% or more, about 2% or more, about 5% or more, about 10% or more, about 15% or more) by mass of the glucose in the culture medium. In particular embodiments, the fatty acid methyl ester is produced at a yield of about 0.5% to about 40%, about 0.5% to about 30%, about 0.5% to about 20%, about 0.5% to about 10%, about 0.5% to about 5%, or about 0.5% to about 4% by mass of the glucose in the culture medium. In particular embodiments, the fatty acid methyl ester is produced at a yield of at least about 0.5%, at least about 4%, at least about 5%, at least about 10%, at least about 20%, at least about 30%, at least about 40%, or at least about 50% by mass of glucose in the culture medium. In particular embodiments, the fatty acid methyl ester is produced at a yield of no more than 50% by mass of glucose in the culture medium.

In some embodiments, the fatty acid methyl ester is produced at a yield of about 10% to about 95% by mass of carbon in the carbon source in the culture medium. For example, the fatty acid methyl ester is produced at a yield of about 10% or more (e.g., about 10% or more, about 20% or more, about 25% or more, about 30% or more, about 35% or more, about 40% or more) by mass of carbon in the carbon source in the culture medium. In particular embodiments, the fatty acid methyl ester is produced at a yield of about 15% to about 90%, about 20% to about 80%, or about 30% to about 70% by mass of carbon in the carbon source in the culture medium. In particular embodiments, the fatty acid methyl ester is produced at a yield of at least about 10%, at least about 20%, at least about 30%, at least about 40%, at least about 50%, at least about 60%, at least about 70%, at least about 80%, at least about 90%, or at least about 95% by mass of carbon in the carbon source in the culture medium. In particular embodiments, the fatty acid methyl ester is produced at a yield of no more than 95% by mass of carbon in the carbon source in the culture medium.

In another aspect, the invention features a fatty ester, such as a fatty acid ester, produced by a method described herein. In some embodiments, the fatty acid ester is a fatty acid methyl ester. In some embodiments, the fatty acid methyl ester is at least about 4, 6, 8, 10, 12, 14, 16, or 18 carbons in length.

In some embodiments, the fatty acid methyl ester comprises an A side and a B side. In some embodiments, the B side of the fatty acid methyl ester is at least about 4, 6, 8, 10, 12, 14, 16, or 18 carbons in length. In some embodiments, the B side of the fatty acid methyl ester includes a straight chain. In other embodiments, the B side of the fatty acid methyl ester includes a branched chain. In still other embodiments, the B side of the fatty acid methyl ester comprises at least one cyclic moiety. In further embodiments, the fatty ester is selected from methyl dedecanoate, methyl 5-dodecenoate, methyl tetradecanoate, methyl 7-tetradecenoate, methyl hexadecanoate, methyl 9-hexadecenoate, methyl octadecanoate, methyl 11-octadecenoate, or combinations thereof. In yet further embodiments, the fatty ester is selected from ethyl dedecanoate, ethyl 5-dodecenoate, ethyl tetradecanoate, ethyl 7-tetradecenoate, ethyl hexadecanoate, ethyl 9-hexadecenoate, ethyl octadecanoate, ethyl 11-octadecenoate, or combinations thereof.

In some embodiments, the fatty acid methyl ester is saturated. In other embodiments, the fatty acid methyl ester is unsaturated. In other embodiments, the fatty acid methyl ester is monounsaturated. In certain embodiments, the fatty acid ethyl ester is saturated. In other embodiments, the fatty acid ethyl ester is unsaturated. In other embodiments, the fatty acid ethyl ester is monounsaturated.

The drawings and examples provided herein are intended solely to illustrate the features of the present invention. They are not intended to be limiting.

### BRIEF DESCRIPTION OF THE DRAWING

FIG. 1 is a diagram illustrating the cloning methods used to generate the plasmid pCLTFWcat.

FIG. 2 is a diagram illustrating the cloning methods used to generate the integration fragment lacZ:: tesA fadD atfA1.

FIG. 3 is the nucleotide sequence of the integration fragment lacZ:: tesA fadD atfA1 .

FIG. 4 lists the nucleotide sequence of the pLacZ plasmid (SEQ ID NO:28)

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DEFINITIONS

The articles “a” and “an” are used herein to refer to one or to more than one (i.e., to at least one) of the grammatical object of the article. By way of example, “an element” means one element or more than one element.

The term “about” is used herein to mean a value±20% of a given numerical value. Thus, “about 60%” means a value of between 60±(20% of 60) (i.e., between 48 and 70).

As used herein, the term “attenuate” means to weaken, reduce, or diminish. For example, a polypeptide can be attenuated by modifying the polypeptide to reduce its activity (e.g., by modifying a nucleotide sequence that encodes the polypeptide).

As used herein, the term “biocrude” refers to a product derived from biomass, biomass derivatives, or other biological sources that, like petroleum crude, can be converted into other fuels. For example, biocrude can be converted into gasoline, diesel, jet fuel, or heating oil. Moreover, biocrude, like petroleum crude, can be converted into other industrially useful chemicals for use in, for example, pharmaceuticals, cosmetics, consumer goods, industrial processes, and the like.

Biocrude may include, for example, hydrocarbons, hydrocarbon products, fatty acid esters, and/or aliphatic ketones. In a preferred embodiment, biocrude is comprised of hydrocarbons, for example aliphatic (e.g., alkanes, alkenes, alkynes) or aromatic hydrocarbons.

As used herein, the term “biodiesel” means a biofuel that can be a substitute of diesel, which is derived from petroleum. Biodiesel can be used in internal combustion diesel engines in either a pure form, which is referred to as “neat” biodiesel, or as a mixture in any concentration with petroleum-based diesel. In one embodiment, biodiesel can include esters or hydrocarbons, such as aldehydes, alkanes, or alkenes.

As used herein, the term “biofuel” refers to any fuel derived from biomass, biomass derivatives, or other biological sources. Biofuels can be substituted for petroleum based fuels. For example, biofuels are inclusive of transportation fuels (e.g., gasoline, diesel, jet fuel, etc.), heating fuels, and electricity-generating fuels. Biofuels are a renewable energy source.

As used herein, the term “biomass” refers to a carbon source derived from biological material. Biomass can be converted into a biofuel. One exemplary source of biomass is plant matter. For example, corn, sugar cane, or switchgrass can be used as biomass. Another non-limiting example of biomass is animal matter, for example cow manure. Biomass also includes waste products from industry, agriculture, forestry, and households. Examples of such waste products that can be used as biomass are fermentation waste, straw, lumber, sewage, garbage, and food leftovers. Biomass also includes sources of carbon, such as carbohydrates (e.g., monosaccharides, disaccharides, or polysaccharides).

As used herein, the phrase “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). These include, for example, various monosaccharides, such as glucose, fructose, mannose, and galactose; oligosaccharides, such as fructo-oligosaccharide and galacto-oligosaccharide; polysaccharides such as xylose and arabinose; disaccharides, such as sucrose, maltose, and turanose; cellulosic material, such as methyl cellulose and sodium carboxymethyl cellulose; saturated or unsaturated fatty acid esters, such as succinate, lactate, and acetate; alcohols, such as methanol, ethanol, propanol, or mixtures thereof. The carbon source can also be a product of photosynthesis, including, but not limited to, glucose. A preferred carbon source is biomass. Another preferred carbon source is glucose.

As used herein, a “cloud point lowering additive” is an additive added to a composition to decrease or lower the cloud point of a solution.

As used herein, the phrase “cloud point of a fluid” means the temperature at which dissolved solids are no longer completely soluble. Below this temperature, solids begin precipitating as a second phase giving the fluid a cloudy appearance. In the petroleum industry, cloud point refers to the temperature below which a solidified material or other heavy hydrocarbon crystallizes in a crude oil, refined oil, or fuel to form a cloudy appearance. The presence of solidified materials influences the flowing behavior of the fluid, the tendency of the fluid to clog fuel filters, injectors, etc., the accumulation of solidified materials on cold surfaces (e.g., a pipeline or heat exchanger fouling), and the emulsion characteristics of the fluid with water.

A nucleotide sequence is “complementary” to another nucleotide sequence if each of the bases of the two sequences matches (e.g., is capable of forming Watson Crick base pairs). The term “complementary strand” is used herein interchangeably with the term “complement”. The complement of a nucleic acid strand can be the complement of a coding strand or the complement of a non-coding strand.

The terms “comprising,” “having,” “including,” and “containing” are to be construed as open-ended terms (e.g., meaning “including, but not limited to,”) unless otherwise noted.

As used herein, the term “conditions sufficient to allow expression” means any conditions that allow a host cell to produce a desired product, such as a polypeptide, aldehyde, or alkane described herein. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source, such as glucose, fructose, cellulose, or the like, that can be metabolized by a host cell directly. In addition, enzymes can be used in the medium to facilitate the mobilization (e.g., the depolymerization of starch or cellulose to fermentable sugars) and subsequent metabolism of the carbon source.

To determine if conditions are sufficient to allow expression, a host cell can be cultured, for example, for about 4, 8, 12, 24, 36, or 48 hours. During and/or after culturing, samples can be obtained and analyzed to determine if the conditions allow expression. For example, the host cells in the sample or the medium in which the host cells were grown can be tested for the presence of a desired product. When testing for the presence of a product, assays, such as, but not limited to, TLC, HPLC, GC/FID, GC/MS, LC/MS, MS, can be used.

It is understood that the polypeptides described herein may have additional conservative or non-essential amino acid substitutions, which do not have a substantial effect on the polypeptide functions. Whether or not a particular substitution will be tolerated (e.g., will not adversely affect desired biological properties, such as decarboxylase activity) can be determined as described in Bowie et al., Science (1990) 247:1306 1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine), and aromatic side chains (e.g., tyrosine, phenylalanine, tryptophan, and histidine).

As used herein, “conditions that permit product production” refers to any fermentation conditions that allow a production host to produce a desired product, such as acyl-CoA or fatty acid derivatives (e.g., fatty acids, hydrocarbons, fatty alcohols, waxes, or fatty esters). Fermentation conditions usually comprise many parameters. Exemplary conditions include, but are not limited to, temperature ranges, levels of aeration, and media composition. Each of these conditions, individually and/or in combination, allows the production host to grow.

Exemplary media include broths and/or gels. Generally, a suitable medium includes a carbon source (e.g., glucose, fructose, cellulose, etc.) that can be metabolized by the microorganism directly. In addition, enzymes can be used in the medium to facilitate the mobilization (e.g., the depolymerization of starch or cellulose to fermentable sugars) and subsequent metabolism of the carbon source.

To determine if the fermentation conditions permit product production, the production host can be cultured for about 4, 8, 12, 24, 36, or 48 hours. During culturing or after culturing, samples can be obtained and analyzed to determine if the fermentation conditions have permitted product production. For example, the production hosts in the sample or the medium in which the production hosts are grown can be tested for the presence of the desired product. Exemplary assays, such as TLC, HPLC, GC/FID, GC/MS, LC/MS, MS, as well as those provided herein, can be used identify and quantify the presence of a product.

As used herein, “control element” means a transcriptional control element. Control elements include promoters and enhancers. The term “promoter element,” “promoter,” or “promoter sequence” refers to a DNA sequence that functions as a switch that activates the expression of a gene. If the gene is activated, it is said to be transcribed or participating in transcription. Transcription involves the synthesis of mRNA from the gene. A promoter, therefore, serves as a transcriptional regulatory element and also provides a site for initiation of transcription of the gene into mRNA. Control elements interact specifically with cellular proteins involved in transcription (Maniatis et al., Science 236:1237, 1987).

As used herein, the term “deletion,” or “knockout” means modifying or inactivating a polynucleotide sequence that encodes a target protein in order to reduce or eliminate the function of the target protein. A polynucleotide deletion can be performed by methods well known in the art (See, e.g., Datsenko et al., Proc. Nat. Acad. Sci. USA, 97:6640-45, 2000 or International Patent Application Nos. PCT/US2007/011923 and PCT/US2008/058788).

As used herein, the term “endogenous” means a polynucleotide that is in the cell and was not introduced into the cell using recombinant genetic engineering techniques. For example, a gene that was present in the cell when the cell was originally isolated from nature. A polynucleotide is still considered endogenous if the control sequences, such as a promoter or enhancer sequences which activate transcription or translation, have been altered through recombinant techniques.

As used herein, the term “ester synthase” means a peptide capable of producing fatty esters. More specifically, an ester synthase is a peptide which converts a thioester to a fatty ester. In a preferred embodiment, the ester synthase converts a thioester (e.g., acyl-CoA) to a fatty ester.

In an alternate embodiment, an ester synthase uses a thioester and an alcohol as substrates to produce a fatty ester. Ester synthases are capable of using short and long chain thioesters as substrates. In addition, ester synthases are capable of using short and long chain alcohols as substrates.

Non-limiting examples of ester synthases are wax synthases, wax-ester synthases, acyl CoA:alcohol transacylases, acyltransferases, and fatty acyl-coenzyme A:fatty alcohol acyltransferases. Exemplary ester synthases are classified in enzyme classification number EC 2.3.1.75. A number of these enzymes, as well as other useful enzymes for making the products described herein, have been disclosed in, for example, International Patent Application Nos. PCT/US2007/011923 and PCT/US2008/058788, which are incorporated herein by reference.

As used herein, the term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. In a preferred embodiment, the fatty acid is made from a fatty acid biosynthetic pathway.

As used herein, the term “fatty acid biosynthetic pathway” means a biosynthetic pathway that produces fatty acids. The fatty acid biosynthetic pathway includes fatty acid enzymes that can be engineered, as described herein, to produce fatty acids, and in some embodiments can be expressed with additional enzymes to produce fatty acids having desired carbon chain characteristics.

As used herein, the term “fatty acid degradation enzyme” means an enzyme involved in the breakdown or conversion of a fatty acid or fatty acid derivative into another product. A nonlimiting example of a fatty acid degradation enzyme is an acyl-CoA synthase. A number of these enzymes, as well as other useful enzymes for making the products described herein, have been disclosed in, for example, International Patent Application Nos. PCT/US2007/011923 and PCT/US2008/058788, which are incorporated herein by reference. Additional examples of fatty acid degradation enzymes are described herein.

As used herein, the term “fatty acid derivative” means products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered as described herein to produce fatty acid derivatives, and in some examples can be expressed with additional enzymes to produce fatty acid derivatives having desired carbon chain characteristics. Exemplary fatty acid derivatives include for example, fatty acids, acyl-CoAs, fatty aldehydes, short and long chain alcohols, hydrocarbons, fatty alcohols, ketones, and esters (e.g., waxes, fatty acid esters, or fatty esters).

As used herein, the term “fatty acid derivative enzymes” means all enzymes that may be expressed or overexpressed in the production of fatty acid derivatives. These enzymes are collectively referred to herein as fatty acid derivative enzymes. These enzymes may be part of the fatty acid biosynthetic pathway. Non-limiting examples of fatty acid derivative enzymes include fatty acid synthases, thioesterases, acyl-CoA synthases, acyl-CoA reductases, alcohol dehydrogenases, alcohol acyltransferases, carboxylic acid reductases, fatty alcohol-forming acyl-CoA reductase, ester synthases, aldehyde biosynthetic polypeptides, and alkane biosynthetic polypeptides. Fatty acid derivative enzymes convert a substrate into a fatty acid derivative. In some examples, the substrate may be a fatty acid derivative which the fatty acid derivative enzyme converts into a different fatty acid derivative. A number of these enzymes, as well as other useful enzymes for making the products described herein, have been disclosed in, for example, International Patent Application Nos. PCT/US2007/011923 and PCT/US2008/058788, which are incorporated herein by reference.

As used herein, “fatty acid enzyme” means any enzyme involved in fatty acid biosynthesis. Fatty acid enzymes can be expressed or overexpressed in host cells to produce fatty acids. Non-limiting examples of fatty acid enzymes include fatty acid synthases and thioesterases. A number of these enzymes, as well as other useful enzymes for making the products described herein, have been disclosed in, for example, International Patent Application Nos. PCT/US2007/011923 and PCT/US2008/058788, which are incorporated herein by reference.

As used herein, the term “fatty ester” means an ester. In a preferred embodiment, a fatty ester is any ester made from a fatty acid to produce, for example, a fatty acid ester. In one embodiment, a fatty ester contains an A side (i.e., the carbon chain attached to the carboxylate oxygen) and a B side (i.e., the carbon chain comprising the parent carboxylate). In a preferred embodiment, when the fatty ester is derived from the fatty acid biosynthetic pathway, the A side is contributed by an alcohol, and the B side is contributed by a fatty acid. Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism that can also produce the fatty acid. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. The B side of the ester is at least about 4, 6, 8, 10, 12, 14, 16, 18, 20, 22, 24, or 26 carbons in length. The A side and/or the B side can be straight or branched chain. The branched chains may have one or more points of branching. In addition, the branched chains may include cyclic branches. Furthermore, the A side and/or B side can be saturated or unsaturated. If unsaturated, the A side and/or B side can have one or more points of unsaturation.

In one embodiment, the fatty ester is produced biosynthetically. In this embodiment, first the fatty acid is “activated.” Non-limiting examples of “activated” fatty acids are acyl-CoA, acyl-ACP, and acyl phosphate. Acyl-CoA can be a direct product of fatty acid biosynthesis or degradation. In addition, acyl-CoA can be synthesized from a free fatty acid, a CoA, or an adenosine nucleotide triphosphate (ATP). An example of an enzyme which produces acyl-CoA is acyl-CoA synthase

After the fatty acid is activated, it can be readily transferred to a recipient nucleophile. Exemplary nucleophiles are alcohols, thiols, or phosphates.

In one embodiment, the fatty ester is a wax. The wax can be derived from a long chain alcohol and a long chain fatty acid. In another embodiment, the fatty ester can be derived from a fatty acyl-thioester and an alcohol. In another embodiment, the fatty ester is a fatty acid thioester, for example fatty acyl Coenzyme A (CoA). In other embodiments, the fatty ester is a fatty acyl panthothenate, an acyl carrier protein (ACP), or a fatty phosphate ester. Fatty esters have many uses. For example, fatty esters can be used as biofuels, surfactants, or formulated into additives that provide lubrication and other benefits to fuels and industrial chemicals.

As used herein, “fraction of modern carbon” or “f_{M}” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (e.g., plant material), f_{M} is approximately 1.1.

The genes “fhuA” and “tonA” are used interchangeably herein.

“Gene knockout”, as used herein, refers to a procedure by which a gene encoding a target protein is modified or inactivated so to reduce or eliminate the function of the intact protein. Inactivation of the gene may be performed by general methods such as mutagenesis by UV irradiation or treatment with N-methyl-N′-nitro-N-nitrosoguanidine, site-directed mutagenesis, homologous recombination, insertion-deletion mutagenesis, or “Red-driven integration” (Datsenko et al., Proc. Natl. Acad. Sci. USA, 97:6640-45, 2000). For example, in one embodiment, a construct is introduced into a host cell, such that it is possible to select for homologous recombination events in the host cell. One of skill in the art can readily design a knock-out construct including both positive and negative selection genes for efficiently selecting transfected cells that undergo a homologous recombination event with the construct. The alteration in the host cell may be obtained, for example, by replacing through a single or double crossover recombination a wild type DNA sequence by a DNA sequence containing the alteration. For convenient selection of transformants, the alteration may, for example, be a DNA sequence encoding an antibiotic resistance marker or a gene complementing a possible auxotrophy of the host cell. Mutations include, but are not limited to, deletion-insertion mutations. An example of such an alteration includes a gene disruption, i.e., a perturbation of a gene such that the product that is normally produced from this gene is not produced in a functional form. This could be due to a complete deletion, a deletion and insertion of a selective marker, an insertion of a selective marker, a frameshift mutation, an in-frame deletion, or a point mutation that leads to premature termination. In some instances, the entire mRNA for the gene is absent. In other situations, the amount of mRNA produced varies.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444 453, algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna. CMP matrix and a gap weight of about 40, 50, 60, 70, or 80 and a length weight of about 1, 2, 3, 4, 5, or 6. A particularly preferred set of parameters (and the one that should be used if the practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5.

Other methods for aligning sequences for comparison are well known in the art. Various programs and alignment algorithms are described in, for example, Smith & Waterman, Adv. Appl. Math. 2:482, 1981; Pearson & Lipman, Proc. Natl. Acad. Sci. USA 85:2444, 1988; Higgins & Sharp, Gene 73:237 244, 1988; Higgins & Sharp, CABIOS 5:151-153, 1989; Corpet et al., Nucleic Acids Research 16:10881-10890, 1988; Huang et al., CABIOS 8:155-165, 1992; and Pearson et al., Methods in Molecular Biology 24:307-331, 1994. and Altschul et al., J. Mol. Biol. 215:403-410, 1990.

As used herein, a “host cell” is a cell used to produce a product described herein (e.g., an aldehyde or alkane). A host cell can be modified to express or overexpress selected genes or to have attenuated expression of selected genes. Non-limiting examples of host cells include plant, animal, human, bacteria, cyanobacteria, yeast, or filamentous fungi cells.

As used herein, the term “hybridizes under low stringency, medium stringency, high stringency, or very high stringency conditions” describes conditions for hybridization and washing. Guidance for performing hybridization reactions can be found, for example, in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified.

The term “isolated” as used herein with respect to nucleic acids, such as DNA or RNA, refers to molecules separated from other DNAs or RNAs, respectively, that are present in the natural source of the nucleic acid. Moreover, an “isolated nucleic acid” includes nucleic acid fragments, such as fragments that are not naturally occurring. The term “isolated” is also used herein to refer to polypeptides, which are isolated from other cellular proteins, and encompasses both purified endogenous polypeptides and recombinant polypeptides. The term “isolated” as used herein also refers to a nucleic acid or polypeptide that is substantially free of cellular material, viral material, or culture medium when produced by recombinant DNA techniques. The term “isolated” as used herein also refers to a nucleic acid or polypeptide that is substantially free of chemical precursors or other chemicals when chemically synthesized.

As used herein, the “level of expression of a gene in a cell” refers to the level of mRNA, pre-mRNA nascent transcript(s), transcript processing intermediates, mature mRNA(s), and/or degradation products encoded by the gene in the cell.

As used herein, the term “microorganism” means prokaryotic and eukaryotic microbial species from the domains Archaea, Bacteria and Eucarya, the latter including yeast and filamentous fungi, protozoa, algae, or higher Protista. The term “microbial cell”, as used herein, means a cell from a microorganism.

As used herein, the term “nucleic acid” refers to a polynucleotide, such as deoxyribonucleic acid (DNA), and, where appropriate, ribonucleic acid (RNA). The term also includes analogs of either RNA or DNA made from nucleotide analogs, and, as applicable to the embodiment being described, single (sense or antisense) and double-stranded polynucleotides, ESTs, chromosomes, cDNAs, mRNAs, and rRNAs. The term “nucleic acid” may be used interchangeably with “polynucleotide,” “DNA,” “nucleic acid molecule,” “nucleotide sequence,” and/or “gene” unless otherwise indicated herein or otherwise clearly contradicted by context.

As used herein, the term “operably linked” means that a selected nucleotide sequence (e.g., encoding a polypeptide described herein) is in proximity with a promoter to allow the promoter to regulate expression of the selected nucleotide sequence. In addition, the promoter is located upstream of the selected nucleotide sequence in terms of the direction of transcription and translation. By “operably linked” is meant that a nucleotide sequence and a regulatory sequence(s) are connected in such a way as to permit gene expression when the appropriate molecules (e.g., transcriptional activator proteins) are bound to the regulatory sequence(s).

The term “or” is used herein to mean, and is used interchangeably with, the term “and/or,” unless context clearly indicates otherwise.

As used herein, “overexpress” means to express or cause to be expressed or produced a nucleic acid, polypeptide, or hydrocarbon in a cell at a greater concentration than is normally expressed in a corresponding wild-type cell. For example, a polypeptide can be “overexpressed” in a recombinant host cell when the polypeptide is present in a greater concentration in the recombinant host cell compared to its concentration in a non-recombinant host cell of the same species.

As used herein, “partition coefficient” or “P,” is defined as the equilibrium concentration of a compound in an organic phase divided by the concentration at equilibrium in an aqueous phase (e.g., fermentation broth). In one embodiment of a bi-phasic system described herein, the organic phase is formed by the aldehyde or alkane during the production process. However, in some examples, an organic phase can be provided, such as by providing a layer of octane, to facilitate product separation. When describing a two phase system, the partition characteristics of a compound can be described as logP. For example, a compound with a logP of 1 would partition 10:1 to the organic phase. A compound with a logP of −1 would partition 1:10 to the organic phase. By choosing an appropriate fermentation broth and organic phase, an organic fatty acid derivative or product with a high logP value can separate into the organic phase even at very low concentrations in the fermentation vessel.

As used herein, the term “polypeptide” may be used interchangeably with “protein,” “peptide,” and/or “enzyme” unless otherwise indicated herein or otherwise clearly contradicted by context.

As used herein, the term “production host” means a cell used to produce the products disclosed herein. The production host is modified to express, overexpress, attenuate or delete expression of selected polynucleotides. Non-limiting examples of production hosts include plant, algal, animal, human, bacteria, yeast, and filamentous fungi cells.

As used herein, the term “purify,” “purified,” or “purification” means the removal or isolation of a molecule from its environment by, for example, isolation or separation. “Substantially purified” molecules are at least about 60% free, preferably at least about 75% free, and more preferably at least about 90% free from other components with which they are associated. As used herein, these terms also refer to the removal of contaminants from a sample. For example, the removal of contaminants can result in an increase in the percentage of a fatty acid derivative or product in a sample. For example, when a fatty acid derivatives or products are produced in a host cell, the fatty acid derivatives or products can be purified by the removal of host cell proteins. After purification, the percentage of fatty acid derivatives or products in the sample is increased.

The terms “purify,” “purified,” and “purification” do not require absolute purity. They are relative terms. Thus, for example, when the fatty acid derivatives or products are produced in host cells, a purified fatty acid derivative or product is one that is substantially separated from other cellular components (e.g., nucleic acids, polypeptides, lipids, carbohydrates, or other fatty acid derivatives or products). In another example, a purified fatty acid derivative or purified product preparation is one in which the fatty acid derivative or product is substantially free from contaminants, such as those that might be present following fermentation. In some embodiments, a fatty acid derivative or product is purified when at least about 50% by weight of a sample is composed of the fatty acid derivative or product. In other embodiments, a fatty acid derivative or product is purified when at least about 60%, 70%, 80%, 85%, 90%, 92%, 95%, 98%, or 99% or more by weight of a sample is composed of the fatty acid derivative or product.

As used herein, the term “recombinant polypeptide” refers to a polypeptide that is produced by recombinant DNA techniques, wherein generally DNA encoding the expressed polypeptide or RNA is inserted into a suitable expression vector and that is in turn used to transform a host cell to produce the polypeptide or RNA.

As used herein, the term “substantially identical” (or “substantially homologous”) is used to refer to a first amino acid or nucleotide sequence that contains a sufficient number of identical or equivalent (e.g., with a similar side chain) amino acid residues (e.g., conserved amino acid substitutions) or nucleotides to a second amino acid or nucleotide sequence such that the first and second amino acid or nucleotide sequences have similar activities.

As used herein, the term “synthase” means an enzyme which catalyzes a synthesis process. As used herein, the term synthase includes synthases, synthetases, and ligases.

As used herein, the term “transfection” means the introduction of a nucleic acid (e.g., via an expression vector) into a recipient cell by nucleic acid-mediated gene transfer.

As used herein, the term “transformation” refers to a process in which a cell's genotype is changed as a result of the cellular uptake of exogenous nucleic acid. This may result in the transformed cell expressing a recombinant form of a RNA or polypeptide. In the case of antisense expression from the transferred gene, the expression of a naturally-occurring form of the polypeptide is disrupted.

As used herein, the term “transport protein” means a polypeptide that facilitates the movement of one or more compounds in and/or out of a cellular organelle and/or a cell. A number of these proteins, as well as other useful proteins for making the products described herein, have been disclosed in, for example, International Patent Application Nos. PCT/US2007/011923 and PCT/US2008/058788, which are incorporated herein by reference.

As used herein, a “variant” of polypeptide X refers to a polypeptide having the amino acid sequence of polypeptide X in which one or more amino acid residues is altered. The variant may have conservative changes or nonconservative changes. Guidance in determining which amino acid residues may be substituted, inserted, or deleted without affecting biological activity may be found using computer programs well known in the art, for example, LASERGENE software (DNASTAR).

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. A splice variant may have significant identity to a reference polynucleotide, but will generally have a greater or fewer number of polynucleotides due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

As used herein, the term “vector” refers to a nucleic acid molecule capable of transporting another nucleic acid to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids,” which refer generally to circular double stranded DNA loops that, in their vector form, are not bound to the chromosome. In the present specification, “plasmid” and “vector” are used interchangeably, as the plasmid is the most commonly used form of vector. However, also included are such other forms of expression vectors that serve equivalent functions and that become known in the art subsequently hereto.

As used herein, the term “wax” means a composition comprised of fatty esters. In a preferred embodiment, the fatty ester in the wax is comprised of medium to long carbon chains. In addition to fatty esters, a wax may comprise other components (e.g., hydrocarbons, sterol esters, aliphatic aldehydes, alcohols, ketones, beta-diketones, triacylglycerols, etc.).

Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides that have the same activity (e.g., that catalyze the same fundamental chemical reaction).

Unless otherwise indicated, the accession numbers referenced herein are derived from the NCBI database (National Center for Biotechnology Information) maintained by the National Institute of Health, U.S.A. Unless otherwise indicated, the accession numbers are as provided in the database as of October 2009.

EC numbers are established by the Nomenclature Committee of the International Union of Biochemistry and Molecular Biology (NC-IUBMB) (available at http://www.chem.qmul.ac.uk/iubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of October 2009.

Unless otherwise defined, all technical and scientific terms used herein have the same meaning as commonly understood by one of ordinary skill in the art to which this invention belongs.

Although methods and materials similar or equivalent to those described herein can be used in the practice or testing of the present invention, suitable methods and materials are described below. All methods described herein can be performed in any suitable order unless otherwise indicated herein or otherwise clearly contradicted by context.

Unless otherwise stated, amounts listed in percentage (%) are in weight percent, based on the total weight of the composition.

All publications, patent applications, patents, and other references mentioned herein are incorporated by reference in their entirety. In case of conflict, the present specification, including definitions, will control. In addition, the materials, methods, and examples are illustrative only and not intended to be limiting.

Recitation of ranges of values herein are merely intended to serve as a shorthand method of referring individually to each separate value falling within the range, unless otherwise indicated herein, and each separate value is incorporated into the specification as if it were individually recited herein.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Other features and advantages of the invention will be apparent from the following detailed description and from the claims.

### Fatty Esters

This disclosure relates to the production of fatty esters, such as fatty acid esters including, for example fatty acid methyl esters (“FAME”) and fatty acid ethyl esters (“FAEE”), in host cells. In particular embodiments, the methods described herein are used to produce fatty acid methyl esters, which can be used in biodiesels.

Fatty esters produced by the methods described herein are not limited to esters of any particular length or other characteristics. For example, a microorganism can be genetically engineered to produce any of the fatty esters described in Knothe, Fuel Processing Technology 86:1059-1070 (2005), using the teachings provided herein. Such fatty esters can be characterized, for example, by centane number (CN), viscosity, melting point, and heat of combustion, as described by Knothe.

Fatty esters that are produced in accordance with the methods, cells, or microorganisms herein comprise, consist essentially of, or consist of the following formula: BCOOA, having an A side and a B side, where the “A side” refers to the carbon chain attached to the carboxylate oxygen of the ester, and the “B side” refers to the carbon chain comprising the parent carboxylate of the ester. The A side is contributed by an alcohol, such as a fatty alcohol, and the B side is contributed by an acid, such as a fatty acid. B is an aliphatic group. In some embodiments, B is a carbon chain. In some embodiments, B comprises a carbon chain that is at least 5, 6, 7, 8, 9, 10, 11, 12, 13, 14, 15, 16, 17, 18, 19, 20, 21, 22, 23, 24, 25, 26, 27, 28, 29, or 30 carbons in length. A comprises at least one carbon atom. In some embodiments, A is an aliphatic group. In some embodiments, A is an alkyl group. In some embodiments, the alkyl group comprises, consists essentially of, or consists of 1, 2, 3, 4, 5, 6, 7, 8, 9, 10, 11, 12, 13, 14, 15, 16, 17, 18, 19, or 20 carbon atoms. In some embodiments, any of the above B groups can be combined with any of the above A groups. In some embodiments, A comprises, consists essentially of, or consists of a carbon chain having a number of carbons selected from the group consisting of 1, 2, 3, 4, and 5 carbon atoms, while B comprises, consists essentially of, or consists of at least 12, 13, 14, 15, 16, 17, 18, 19, or 20 carbon atoms.

In some embodiments, the fatty esters of the invention comprise a plurality of individual fatty esters. In some embodiments, the methods described herein permit production of a plurality of fatty esters of varied length. In some embodiments, the fatty ester product comprises saturated or unsaturated fatty esters product(s) having a carbon atom content limited to between 5 and 25 carbon atoms. In other words, the invention provides a composition comprising C₅-C₂₅ fatty esters (e.g., C₁₀-C₂₀ fatty esters, or C₁₂-C₁₈ fatty esters).

In some embodiments, the fatty esters comprise one or more fatty esters having a double bond at one or more points in the carbon chain. Thus, in some embodiments, a 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, or 30-carbon chain can have 1, 2, 3, 4, 5, 6, 7, 8, 9, 10, 11, 12, 13, 14, 15, 16, 17, 18, 19, 20, 21, 22, 23, or 24 double bonds, and 1-24 of the aforesaid double bonds can be located following carbon 1, 2, 3, 4, 5, 6, 7, 8, 9, 10, 11, 12, 13, 14, 15, 16, 17, 18, 19, 20, 21, 22, 23, 24, 25, 26, 27, 28, or 29. In some embodiments, a 1-, 2-, 3-, 4-, or 5-carbon chain for A can have 1, 2, 3, or 4 double bonds and 1-4 of the double bonds can be located following carbon 1, 2, 3, or 4. In some embodiments, any of the above A groups can be combined with any of the above B groups.

In certain preferred embodiments, the B group can have 12, 13, 14, 15, 16, 17, 18 carbon atoms in a chain. In other embodiments, the A group can have one or two carbon atoms.

In some preferred embodiments, the B group can have one double bond at one or more points in the carbon chain. In more preferred embodiments, the B group can have one double bond at position 7 of the carbon chain, numbering from the reduced end of the carbon chain. One of ordinary skill in the art will recognize that one end of the B group will have a methyl group, and the other end of the B group will have a carboxyl group (C(═O)O—). The end of the B group which is a methyl group is the reduced end of the carbon chain comprising the B group, thus, the double bond is at carbon 7 counting from the methyl group terminus of the B group (e.g., at between carbons 7 and 8 of the B group). The double bond can have any geometry, thus, the double bond in the B group can be cis or trans.

In some embodiments, the fatty esters comprise straight chain fatty esters. In some embodiments, the fatty esters comprise branched chain fatty esters. In some embodiments, the fatty esters comprise cyclic moieties.

In certain preferred embodiments, the fatty esters can be selected from the group consisting of methyl dodecanoate, methyl 5-dodecenoate, methyl tetradecanoate, methyl 7-tetradecenoate, methyl hexadecanoate, methyl 9-hexadecenoate, methyl octadecanoate, methyl 11-octadecenoate, and combinations thereof.

In some embodiments, the fatty ester composition comprises about 5 wt. % or more methyl dedecanoate. In some embodiments, the fatty ester composition comprises about 25% or more methyl dedecanoate. In some embodiments, the fatty ester composition comprises about 5 wt. % to about 25 wt. % methyl dodecanoate.

In some embodiments, the fatty ester composition comprises about 10 wt. % or less methyl dodec-7-enoate. In some embodiments, the fatty ester composition comprises about 0 wt. % to about 10 wt. % methyl dodec-7-enoate.

In some embodiments, the fatty ester composition comprises about 30 wt. % or more methyl tetradecanoate. In some embodiments, the fatty ester composition comprises about 50 wt. % or less methyl tetradecanoate. In some embodiments, the fatty ester composition comprises about 30 wt. % to about 50 wt. % methyl tetradecanoate.

In some embodiments, the fatty ester composition comprises about 10 wt. % or less methyl tetradec-7-enoate. In some embodiments, the fatty ester composition comprises about 0 wt. % to about 10 wt. % methyl tetradec-7-enoate.

In some embodiments, the fatty ester composition comprises about 15 wt. % or less methyl hexadecanoate. In some embodiments, the fatty ester composition comprises about 0 wt. % to about 15 wt. % methyl hexadecanoate.

In some embodiments, the fatty ester composition comprises about 10 wt. % or more methyl hexadec-7-enoate. In some embodiments, the fatty ester composition comprises about 40 wt. % or less methyl hexadec-7-enoate. In some embodiments, the fatty ester composition comprises about 10 wt. % to about 40 wt. % methyl hexadec-7-enoate.

In some embodiments, the fatty ester composition comprises about 15 wt. % or less methyl octadec-7-enoate. In some embodiments, the fatty ester composition comprises about 0 wt. % to about 15 wt. % methyl octadec-7-enoate.

These exemplary fatty ester products, having characteristic features of A side and/or B side, can be prepared and produced using substrates having similar or the same features. Accordingly, each step within a biosynthetic pathway that leads to the production of a fatty acid derivative can be modified to produce or overproduce a substrate leading to a fatty alcohol and/or a fatty acid. For example, known genes involved in the fatty acid biosynthetic pathway or the fatty alcohol pathway can be expressed, overexpressed, or attenuated in host cells to produce a desired substrate (see, e.g., WO2008/119082, the disclosure of which is incorporated by reference herein).

### Synthesis of Substrates

Fatty acid synthase (FAS) is a group of polypeptides that catalyze the initiation and elongation of acyl chains (Marrakchi et al., Biochemical Society, 30:1050-1055, 2002). The acyl carrier protein (ACP) along with the enzymes in the FAS pathway control the length, degree of saturation, and branching of the fatty acid derivatives produced. The fatty acid biosynthetic pathway involves the precursors acetyl-CoA and malonyl-CoA. The steps in this pathway are catalyzed by enzymes of the fatty acid biosynthesis (fab) and acetyl-CoA carboxylase (acc) gene families (see, e.g., Heath et al., Prog. Lipid Res. 40(6):467-97 (2001)).

Host cells can be engineered to express fatty acid derivative substrates by recombinantly expressing or overexpressing one or more fatty acid synthase genes, such as acetyl-CoA and/or malonyl-CoA synthase genes. For example, to increase acetyl-CoA production, one or more of the following genes can be expressed in a host cell: pdh (a multienzyme complex comprising aceEF (which encodes the Elp dehydrogenase component, the E2p dihydrolipoamide acyltransferase component of the pyruvate and 2-oxoglutarate dehydrogenase complexes, and lpd), panK, fabH, fabB, fabD, fabG, acpP, and fabF. Exemplary GenBank accession numbers for these genes are: pdh (BAB34380, AAC73227, AAC73226), panK (also known as CoA, AAC76952), aceEF (AAC73227, AAC73226), fabH (AAC74175), fabB (P0A953), fabD (AAC74176), fabG (AAC74177), acpP (AAC74178), fabF (AAC74179). Additionally, the expression levels of fadE, gpsA, ldhA, pflb, adhE, pta, poxB, ackA, and/or ackB can be attenuated or knocked-out in an engineered host cell by transformation with conditionally replicative or non-replicative plasmids containing null or deletion mutations of the corresponding genes or by substituting promoter or enhancer sequences. Exemplary GenBank accession numbers for these genes are: fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430). The resulting host cells will have increased acetyl-CoA production levels when grown in an appropriate environment.

Malonyl-CoA overexpression can be affected, for example, by introducing one or more or all subunits of a four-subunit protein accABCD (e.g., accession number AAC73296, EC 6.4.1.2) into a host cell. Fatty acids can be further produced in host cells by introducing into the host cell a DNA sequence encoding a lipase (e.g., accession numbers CAA89087, CAA98876).

In addition, inhibiting PlsB can lead to an increase in the levels of long chain acyl-ACP, which will inhibit early steps in the pathway (e.g., the expression of accABCD, fabH, or fabI). The plsB (e.g., accession number AAC77011) D311E mutation can be used to increase the amount of available fatty acids.

In addition, a host cell can be engineered to overexpress a sfa gene (suppressor of fabA, e.g., accession number AAN79592) to increase production of monounsaturated fatty acids (Rock et al., J. Bacteriology 178:5382-5387 (1996)).

The chain length of a fatty acid derivative substrate can be selected for by modifying the expression of a thioesterase, which influences the chain length of fatty acids produced. Hence, host cells can be engineered to express, overexpress, have attenuated expression, or not to express one or more selected thioesterases to increase the production of a preferred fatty acid derivative substrate. For example, C₁₀ fatty acids can be produced by expressing a thioesterase that has a preference for producing C₁₀ fatty acids and attenuating thioesterases that have a preference for producing fatty acids other than C₁₀ fatty acids (e.g., a thioesterase which prefers to produce C₁₄ fatty acids). This would result in a relatively homogeneous population of fatty acids that have a carbon chain length of, for example, 10. In other instances, C₁₄ fatty acids can be produced by attenuating endogenous thioesterases that produce non-C₁₄ fatty acids and expressing the thioesterases that use C₁₄-ACP. In some situations, C₁₂ fatty acids can be produced by expressing thioesterases that use C₁₂-ACP, while in parallel, attenuating thioesterases that produce non-C₁₂ fatty acids. Acetyl-CoA, malonyl-CoA, and fatty acid overproduction can be verified using methods known in the art, for example, by using radioactive precursors, HPLC, or GC-MS subsequent to cell lysis. Non-limiting examples of thioesterases that can be used in the methods described herein are listed in Table 1.

TABLE 1
Thioesterases

| Accession Number | Source Organism | Gene |
|---|---|---|
| AAC73596 | E. coli | tesA without |
| | | leader sequence |
| AAC73555 | E. coli | tesB |
| Q41635, AAA34215 | Umbellularia california | fatB |
| AAC49269 | Cuphea hookeriana | fatB2 |
| Q39513; AAC72881 | Cuphea hookeriana | fatB3 |
| Q39473, AAC49151 | Cinnamonum camphorum | fatB |
| CAA85388 | Arabidopsis thaliana | fatB [M141T]* |
| NP 189147; NP 193041 | Arabidopsis thaliana | fatA |
| CAC39106 | Bradyrhiizobium japonicum | fatA |
| AAC72883 | Cuphea hookeriana | fatA |
| AAL79361 | Helianthus annus | fatA1 |

*Mayer et al., BMC Plant Biology 7: 1-11, (2007)

In other instances, fatty esters are produced in a host cell that contains a naturally occurring mutation that results in an increased level of fatty acids in the host cell. In some instances, the host cell is genetically engineered to increase the level of fatty acids in the host cell relative to a corresponding wild-type host cell. For example, the host cell can be genetically engineered to express a reduced or attenuated level of an acyl-CoA synthase relative to a corresponding wild-type host cell. In a particular embodiment, the level of expression of one or more genes (e.g., an acyl-CoA synthase gene) can be eliminated by genetically engineering a “knock out” host cell, wherein the one or more genes are deleted.

Any known acyl-CoA synthase gene can be reduced or knocked out in a host cell. Non-limiting examples of acyl-CoA synthase genes include fadD, fadK, BH3103, yhfL, Pfl-4354, EAV15023, fadD1, fadD2, RPC_{—}4074, fadDD35, fadDD22, faa3p or the gene encoding the protein ZP_{—}01644857. Specific examples of acyl-CoA synthase genes include fadDD35 from M. tuberculosis H37Rv [NP_{—}217021], fadDD22 from M. tuberculosis H37Rv [NP_{—}217464],fadD from E. coli [NP_{—}416319], fadK from E. coli [YP_{—}416216], fadD from Acinetobacter sp. ADP1 [YP_{—}045024],fadD from Haemophilus influenza RdkW20 [NP_{—}438551], fadD from Rhodopseudomonas palustris Bis B18 [YP_{—}533919], BH3101 from Bacillus halodurans C-125 [NP_{—}243969], Pfl-4354 from Pseudomonas fluorescens Pfo-1 [YP_{—}350082], EAV15023 from Comamonas testosterone KF-1 [ZP_{—}01520072], yhfL from B. subtilis [NP_{—}388908],fadD1 from P. aeruginosa PAO1 [NP_{—}251989],fadD1 from Ralstonia solanacearum GM1 1000 [NP_{—}520978],fadD2 from P. aeruginosa PAO1 [NP_{—}251990], the gene encoding the protein ZP_{—}01644857 from Stenotrophomonas maltophilia R551-3, faa3p from Saccharomyces cerevisiae [NP_{—}012257], faa1p from Saccharomyces cerevisiae [NP_{—}014962], lcfA from Bacillus subtilis [CAA99571], or those described in Shockey et al., Plant. Physiol. 129:1710-1722 (2002); Caviglia et al., J. Biol. Chem. 279:1163-1169 (2004); Knoll et al., J. Biol. Chem. 269(23):16348-56 (1994); Johnson et al., J. Biol. Chem. 269: 18037-18046 (1994); and Black et al., J. Biol Chem. 267: 25513-25520 (1992).

### Formation of Branched Fatty Esters

Fatty esters can be produced that contain branch points by using branched fatty acid derivatives as substrates or precursors. For example, although E. coli naturally produces straight chain fatty acids (sFAs), E. coli can be engineered to produce branched chain fatty acids (brFAs) by introducing and expressing or overexpressing genes that provide branched precursors in the E. coli (e.g., bkd, ilv, icm, and fab gene families). Additionally, a host cell can be engineered to express or overexpress one or more genes encoding one or more proteins for the elongation of brFAs (e.g., ACP, FabF, etc.) and/or to delete or attenuate the corresponding host cell genes that normally lead to sFAs.

The first step in forming brFAs is the production of the corresponding α-keto acids by a branched-chain amino acid aminotransferase. Host cells may endogenously include genes encoding such enzymes or such genes can be recombinantly introduced. E. coli, for example, endogenously expresses such an enzyme, INE (EC 2.6.1.42; GenBank accession YP_{—}026247). In some host cells, a heterologous branched-chain amino acid aminotransferase may not be expressed. However, E. coli IlvE or any other branched-chain amino acid aminotransferase (e.g., IlvE from Lactococcus lactis (GenBank accession AAF34406), IlvE from Pseudomonas putida (GenBank accession NP_{—}745648), or IlvE from Streptomyces coelicolor (GenBank accession NP_{—}629657)), if not endogenous, can be introduced.

In another embodiment, the production of α-keto acids can be achieved by using the methods described in Atsumi et al., Nature 451:86-89, 2008. For example, 2-ketoisovalerate can be produced by overexpressing the genes encoding IlvI, INH, IlvC, or IlvD. In another example, 2-keto-3-methyl-valerate can be produced by overexpressing the genes encoding IlvA and IlvI, IlvH (or AlsS of Bacillus subtilis), INC, IlvD, or their corresponding homologs. In a further embodiment, 2-keto-4-methyl-pentanoate can be produced by overexpressing the genes encoding IlvI, IlvH, IlvC, IlvD and LeuA, LeuB, LeuC, LeuD, or their corresponding homologs.

The second step is the oxidative decarboxylation of the α-keto acids to the corresponding branched-chain acyl-CoA. This reaction can be catalyzed by a branched-chain α-keto acid dehydrogenase complex (bkd; EC 1.2.4.4.) (Denoya et al., J. Bacteriol. 177:3504, 1995), which consists of E1α/β (decarboxylase), E2 (dihydrolipoyl transacylase), and E3 (dihydrolipoyl dehydrogenase) subunits. These branched-chain α-keto acid dehydrogenase complexes are similar to pyruvate and α-ketoglutarate dehydrogenase complexes. Any microorganism that possesses brFAs and/or grows on branched-chain amino acids can be used as a source to isolate bkd genes for expression in host cells, for example, E. coli. Furthermore, E. coli has the E3 component as part of its pyruvate dehydrogenase complex (lpd, EC 1.8.1.4, GenBank accession NP_{—}414658). Thus, it may be sufficient to express only the E1 α/β and E2 bkd genes. Table 2 lists non-limiting examples of bkd genes from several microorganisms that can be recombinantly introduced and expressed in a host cell to provide branched-chain acyl-CoA precursors.

TABLE 2
Bkd genes from selected microorganisms

| | | GenBank |
|---|---|---|
| Organism | Gene | Accession # |
| Streptomyces coelicolor | bkdA1 (E1α) | NP_628006 |
| | bkdB1 (E1β) | NP_628005 |
| | bkdC1 (E2) | NP_638004 |
| Streptomyces coelicolor | bkdA2 (E1α) | NP_733618 |
| | bkdB2 (E1β) | NP_628019 |
| | bkdC2 (E2) | NP_628018 |
| Streptomyces avermitilis | bkdA (E1a) | BAC72074 |
| | bkdB (E1b) | BAC72075 |
| | bkdC (E2) | BAC72076 |
| Streptomyces avermitilis | bkdF (E1α) | BAC72088 |
| | bkdG (E1β) | BAC72089 |
| | bkdH (E2) | BAC72090 |
| Bacillus subtilis | bkdAA (E1α) | NP_390288 |
| | bkdAB (E1β) | NP_390288 |
| | bkdB (E2) | NP_390288 |
| Pseudomonas putida | bkdA1 (E1α) | AAA65614 |
| | bkdA2 (E1β) | AAA65615 |
| | bkdC (E2) | AAA65617 |

In another example, isobutyryl-CoA can be made in a host cell, for example in E. coli, through the coexpression of a crotonyl-CoA reductase (Ccr, EC 1.6.5.5, 1.1.1.1) and isobutyryl-CoA mutase (large subunit IcmA, EC 5.4.99.2; small subunit IcmB, EC 5.4.99.2) (Han and Reynolds, J. Bacteriol. 179:5157 (1997)). Crotonyl-CoA is an intermediate in fatty acid biosynthesis in E. coli and other microorganisms. Non-limiting examples of ccr and icm genes from selected microorganisms are listed in Table 3.

TABLE 3
Ccr and icm genes from selected microorganisms

| | | GenBank |
|---|---|---|
| Organism | Gene | Accession # |
| Streptomyces coelicolor | ccr | NP_630556 |
| | icmA | NP_629554 |
| | icmB | NP_630904 |
| Streptomyces cinnamonensis | ccr | AAD53915 |
| | icmA | AAC08713 |
| | icmB | AJ246005 |

In addition to expression of the bkd genes, the initiation of brFA biosynthesis utilizes β-ketoacyl-acyl-carrier-protein synthase III (FabH, EC 2.3.1.41) with specificity for branched chain acyl-CoAs (Li et al., J. Bacteriol. 187:3795-3799 (2005)). Non-limiting examples of such FabH enzymes are listed in Table 4. fabH genes that are involved in fatty acid biosynthesis of any brFA-containing microorganism can be expressed in a host cell. The Bkd and FabH enzymes from host cells that do not naturally make brFA may not support brFA production. Therefore, bkd and fabH can be expressed recombinantly. Vectors containing the bkd and fabH genes can be inserted into such a host cell. Similarly, the endogenous level of Bkd and FabH production may not be sufficient to produce brFA. In this case, they can be overexpressed. Additionally, other components of the fatty acid biosynthesis pathway can be expressed or overexpressed, such as acyl carrier proteins (ACPs) and β-ketoacyl-acyl-carrier-protein synthase II (fabF, EC 2.3.1.41) (non-limiting examples of FabH, ACP, and fabF genes from select microorganisms are listed in Table 4). In addition to expressing these genes, some genes in the endogenous fatty acid biosynthesis pathway can be attenuated in the host cell (e.g., the E. coli genes fabH (GenBank accession #NP_{—}415609) and/or fabF (GenBank accession #NP_{—}415613)).

TABLE 4
FabH, ACP and fabF genes from selected
microorganisms with brFAs

| | | GenBank |
|---|---|---|
| Organism | Gene | Accession # |
| Streptomyces coelicolor | fabH1 | NP_626634 |
| | acp | NP_626635 |
| | fabF | NP_626636 |
| Streptomyces avermitilis | fabH3 | NP_823466 |
| | fabC3 (acp) | NP_823467 |
| | fabF | NP_823468 |
| Bacillus subtilis | fabH_A | NP_389015 |
| | fabH_B | NP_388898 |
| | acp | NP_389474 |
| | fabF | NP_389016 |
| Stenotrophomonas | SmalDRAFT_0818 (fabH) | ZP_01643059 |
| maltophilia | SmalDRAFT_0821 (acp) | ZP_01643063 |
| | SmalDRAFT_0822 (fabF) | ZP_01643064 |
| Legionella pneumophila | fabH | YP_123672 |
| | acp | YP_123675 |
| | fabF | YP_123676 |

### Formation of Cyclic Fatty Esters

Cyclic fatty esters can be produced by using cyclic fatty acid derivatives as substrates. To produce cyclic fatty acid derivative substrates, genes that provide cyclic precursors (e.g., the ans, chc, and plm gene families) can be introduced into the host cell and expressed to allow initiation of fatty acid biosynthesis from cyclic precursors. For example, to convert a host cell, such as E. coli, into one capable of synthesizing ω-cyclic fatty acids (cyFA), a gene that provides the cyclic precursor cyclohexylcarbonyl-CoA (CHC-CoA) (Cropp et al., Nature Biotech. 18:980-983 (2000)) can be introduced and expressed in the host cell. Non-limiting examples of genes that provide CHC-CoA in E. coli include: ansJ, ansK, ansL, chcA, and ansM from the ansatrienin gene cluster of Streptomyces collinus (Chen et al., Eur. J. Biochem. 261: 98-107 (1999)) or plmJ, plmK, plmL, chcA, and plmM from the phoslactomycin B gene cluster of Streptomyces sp. HK803 (Palaniappan et al., J. Biol. Chem. 278:35552-35557 (2003)) together with the chcB gene (Patton et al., Biochem. 39:7595-7604 (2000)) from S. collinus, S. avermitilis, or S. coelicolor (see Table 5). Furthermore, the genes listed in Table 4, which tend to have broad substrate specificity, can then be expressed to allow initiation and elongation of w-cyclic fatty acids. Alternatively, the homologous genes can be isolated from microorganisms that make cyFA and expressed in a host cell (e.g., E. coli).

TABLE 5
Genes for the synthesis of CHC-CoA

| | | GenBank |
|---|---|---|
| Organism | Gene | Accession # |
| Streptomyces collinus | ansJK | U72144* |
| | ansL | |
| | chcA | |
| | ansM | |
| | chcB | AF268489 |
| Streptomyces sp. HK803 | pmlJK | AAQ84158 |
| | pmlL | AAQ84159 |
| | chcA | AAQ84160 |
| | pmlM | AAQ84161 |
| Streptomyces coelicolor | chcB/caiD | NP_629292 |
| Streptomyces avermitilis | chcB/caiD | NP_629292 |

*Only chcA is annotated in GenBank entry U72144, ansJKLM are according to Chen et al. (Eur. J. Biochem. 261: 98-107, (1999)).

The genes listed in Table 4 (fabH, acp, and fabF) allow initiation and elongation of w-cyclic fatty acids because they have broad substrate specificity. If the coexpression of any of these genes with the genes listed in Table 5 does not yield cyFA, then fabH, acp, and/or fabF homologs from microorganisms that make cyFAs (e.g., those listed in Table 6) can be isolated (e.g., by using degenerate PCR primers or heterologous DNA sequence probes) and coexpressed.

TABLE 6
Non-limiting examples of microorganisms
that contain ω-cyclic fatty acids

| Organism | Reference |
|---|---|
| Curtobacterium pusillum | ATCC19096 |
| Alicyclobacillus acidoterrestris | ATCC49025 |
| Alicyclobacillus acidocaldarius | ATCC27009 |
| Alicyclobacillus cycloheptanicus * | Moore, J. Org. Chem. 62: pp. |
| | 2173, 1997 |

* Uses cycloheptylcarbonyl-CoA and not cyclohexylcarbonyl-CoA as precursor for cyFA biosynthesis.

### Fatty Ester Saturation Levels

The degree of saturation in fatty acids can be controlled by regulating the degree of saturation of fatty acid intermediates. For example, the sfa, gns, and fab families of genes can be expressed, overexpressed, or expressed at reduced levels, to control the saturation of fatty acids. A number of those genes have been described in, for example, WO 2008/119082, the disclosure of which is incorporated by reference. Non-limiting examples of genes in these gene families include GenBank Accession No: AAN79592, AAC44390, ABD18647.1, AAC74076.1, BAA16180, AAF98273, AAU39821, or DDA05501.

For example, host cells can be engineered to produce unsaturated fatty acids by engineering the production host to overexpress fabB or by growing the production host at low temperatures (e.g., less than 37° C.). FabB has preference to cis-δ3decenoyl-ACP and results in unsaturated fatty acid production in E. coli. Overexpression of fabB results in the production of a significant percentage of unsaturated fatty acids (de Mendoza et al., J. Biol. Chem. 258:2098-2101 (1983)). In some embodiments, an endogenous fabB gene may be modified such that it is overexpressed. In some other embodiments, a heterologous fabB gene may be inserted into and expressed in host cells not naturally having the gene. These unsaturated fatty acids can then be used as intermediates in host cells that are engineered to produce fatty acid derivatives, such as fatty esters.

In other instances, a repressor of fatty acid biosynthesis, for example, fabR (GenBank accession NP_{—}418398), can be deleted, which will also result in increased unsaturated fatty acid production in E. coli (Zhang et al., J. Biol. Chem. 277:15558, (2002)). Similar deletions may be made in other host cells. A further increase in unsaturated fatty acids may be achieved, for example, by overexpressing fabM (trans-2, cis-3-decenoyl-ACP isomerase, GenBank accession DAA05501) and controlled expression of fabK (trans-2-enoyl-ACP reductase II, GenBank accession NP_{—}357969) from Streptococcus pneumoniae (Marrakchi et al., J. Biol. Chem. 277: 44809 (2002)), while deleting E. coli fabI (trans-2-enoyl-ACP reductase, GenBank accession NP_{—}415804). In some examples, the endogenous fabF gene can be attenuated, thus increasing the percentage of palmitoleate (C16:1) produced.

In yet other examples, host cells can be engineered to produce saturated fatty acids by reducing the expression of an sfa, gns, or fab gene.

For example, a host cell can be engineered to express a decreased level of fabA and/or fabB. In some instances, the host cell can be grown in the presence of unsaturated fatty acids. In other instances, the host cell can be further engineered to express or overexpress a gene encoding a desaturase enzyme. One non-limiting example of a desaturase is B. subtilis DesA (AF037430). Other genes encoding desaturase enzymes are known in the art and can be used in the host cells and methods described herein, such as desaturases that use acyl-ACP, such as hexadecanoyl-ACP or octadecanoyl-ACP.

### Ester Synthases

Fatty esters can be synthesized by acyl-CoA:fatty alcohol acyltransferase, which conjugates an alcohol to a fatty acyl-CoA via an ester linkage. Ester synthases and encoding genes are known from the jojoba plant and the bacterium Acinetobacter sp. strain ADP1 (formerly Acinetobacter calcoaceticus ADP1). The bacterial ester synthase is a bifunctional enzyme, exhibiting ester synthase activity and the ability to form triacylglycerols from diacylglycerol substrates and fatty acyl-CoAs (acyl-CoA:diglycerol acyltransferase (DGAT) activity). The gene wax/dgat encodes both ester synthase and DGAT. See Cheng et al., J. Biol. Chem. 279(36):37798-37807 (2004); Kalscheuer and Steinbuchel, J. Biol. Chem. 278:8075-8082 (2003).

Other ester synthases (EC 2.3.1.20, 2.3.1.75) are known in the art, and exemplary GenBank Accession Numbers include, without limitation, NP_{—}190765, AAA16514, AAF19262, AAX48018, AA017391, or AAD38041. Methods to identify ester synthase activity are provided in, e.g., U.S. Pat. No. 7,118,896, which is herein incorporated by reference in its entirety. Other ester synthases include bifunctional ester synthase/acyl-CoA:diacylglycerol acyltransferases, nonlimiting examples of which include the multienzyme complexes from Simmondsia chinensis, Acinetobacter sp. strain ADP1 (formerly Acinetobacter calcoaceticus ADP1), Alcanivorax borkumensis, Pseudomonas aeruginosa, Fundibacter jadensis, Arabidopsis thaliana, or Alcaligenes eutrophus (later renamed Ralstonia eutropha). In one embodiment, the fatty acid elongases, acyl-CoA reductases or wax synthases can be from a multienzyme complex from Alcaligenes eutrophus (later renamed Ralstonia eutropha) or other organisms known in the literature to produce esters, such as wax or fatty esters. Additional sources of heterologous DNA sequence encoding ester synthesis proteins that can be used in fatty ester production include, but are not limited to, Mortierella alpina (e.g., ATCC 32222), Cryptococcus curvatus (also referred to as Apiotricum curvatum), Alcanivorax jadensis (for example T9T=DSM 12718=ATCC 700854), Acinetobacter sp. HO1-N, (e.g., ATCC 14987), Rhodococcus opacus (e.g., PD630, DSMZ 44193), and the ester synthases from Marinobacter hydrocarbonoclastics (e.g., DSM 8798).

### Genetic Engineering of Host Cells to Produce Fatty Esters

Various host cells can be used to produce fatty esters, as described herein. A host cell can be any prokaryotic or eukaryotic cell. For example, a polypeptide described herein can be expressed in bacterial cells (such as E. coli), insect cells, yeast, or mammalian cells (such as Chinese hamster ovary cells (CHO) cells, COS cells, VERO cells, BHK cells, HeLa cells, Cvl cells, MDCK cells, 293 cells, 3T3 cells, or PC12 cells). Other exemplary host cells include cells from the members of the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Schizosaccharomyces, Yarrowia, or Streptomyces. Yet other exemplary host cells can be a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhizomucor miehei cell, a Mucor michei cell, a Streptomyces lividans cell, a Streptomyces murinus cell, or an Actinomycetes cell. Other host cells are cyanobacterial host cells.

In certain embodiments, the host cell is an Actinomycetes, Saccharomyces cerevisiae, Candida lipolytica (or Yarrowia lipolytica), E. coli, Arthrobacter AK 19, Rhodotorula glutinims, Acintobacter sp. M-1 cell, or a cell from other oleaginous microorganisms.

In particular embodiments, the host cell is a cell from an eukaryotic plant, algae, cyanobacterium, green-sulfur bacterium, green non-sulfur bacterium, purple sulfur bacterium, purple non-sulfur bacterium, extremophile, yeast, fungus, engineered organisms thereof, or a synthetic organism. In some embodiments, the host cell is light dependent or fixes carbon. In some embodiments, the host cell has autotrophic activity. In some embodiments, the host cell has photoautotrophic activity, such as in the presence of light. In certain embodiments, the host cell is a cell from Arabidopsis thaliana, Panicum virgatums, Miscanthus giganteus, Zea mays, botryococcuse braunii, Chalamydomonas reinhardtii, Dunaliela salina, Thermosynechococcus elongatus, Chlorobium tepidum, Chloroflexus auranticus, Chromatiumm vinosum, Rhodospirillum rubrum, Rhodobacter capsulatus, Rhodopseudomonas palusris, Clostridium ljungdahlii, Clostridiuthermocellum, or Pencillium chrysogenum. In certain other embodiments, the host cell is from Pichia pastories, Saccharomyces cerevisae, Yarrowia lipolytica, Schizosaccharomyces pombe, Pseudomonas fluorescens, or Zymomonas mobilis. In yet further embodiments, the host cell is a cell from Synechococcus sp. PCC 7002, Synechococcus elongatus. PCC 7942, or Synechocystis sp. PCC6803.

In a preferred embodiment, the host cell is an E. coli cell, a Saccharomyces cerevisiae cell, or a Bacillus subtilis cell. In a more preferred embodiment, the host cell is from E. coli strains B, C, K, or W. Other suitable host cells are known to those skilled in the art.

Various methods well known in the art can be used to genetically engineer host cells to produce fatty esters. The methods can include the use of vectors, preferably expression vectors, containing a nucleic acid encoding a fatty acid biosynthetic polypeptide described herein, polypeptide variant, or a fragment thereof. Those skilled in the art will appreciate a variety of viral vectors (for example, retroviral vectors, lentiviral vectors, adenoviral vectors, and adeno-associated viral vectors) and non-viral vectors can be used in the methods described herein.

The recombinant expression vectors described herein include a nucleic acid described herein in a form suitable for expression of the nucleic acid in a host cell. The recombinant expression vectors can include one or more control sequences, selected on the basis of the host cell to be used for expression. The control sequence is operably linked to the nucleic acid sequence to be expressed. Such control sequences are described, for example, in Goeddel, Gene Expression Technology: Methods in Enzymology 185, Academic Press, San Diego, Calif. (1990). Control sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors described herein can be introduced into host cells to produce polypeptides, including fusion polypeptides, encoded by the nucleic acids as described herein.

Recombinant expression vectors can be designed for expression of a fatty acid biosynthetic polypeptide or variant in prokaryotic or eukaryotic cells (e.g., bacterial cells, such as E. coli, insect cells (e.g., using baculovirus expression vectors), yeast cells, or mammalian cells). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example, by using T7 promoter regulatory sequences and T7 polymerase.

Expression of polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech Inc; Smith et al., Gene 67:31-40 (1988)), pMAL (New England Biolabs, Inc., Ipswich, Mass.), and pRITS (Pharmacia, Piscataway, N.J.), which fuse glutathione S-transferase (GST), maltose E binding protein, or protein A, respectively, to the target recombinant polypeptide.

Examples of inducible, non-fusion E. coli expression vectors include pTrc (Amann et al., Gene 69:301-315 (1988)) and pET 11d (Studier et al., Gene Expression Technology: Methods in Enzymology 185, Academic Press, San Diego, Calif. 60-89 (1990)). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gni). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant polypeptide expression is to express the polypeptide in a host cell with an impaired capacity to proteolytically cleave the recombinant polypeptide (see, Gottesman, Gene Expression Technology: Methods in Enzymology 185, Academic Press, San Diego, Calif. 119-128 (1990)). Another strategy is to alter the nucleic acid sequence to be inserted into an expression vector so that the individual codons for each amino acid are those preferentially utilized in the host cell (Wada et al., Nucleic Acids Res. 20:2111-2118 (1992)). Such alteration of nucleic acid sequences can be carried out by standard DNA synthesis techniques.

In another embodiment, the host cell is a yeast cell. In this embodiment, the expression vector is a yeast expression vector. Examples of vectors for expression in yeast S. cerevisiae include pYepSec1 (Baldari et al., EMBO J. (1987) 6:229-234), pMFa (Kurjan et al., Cell 30:933-943 (1982)), pJRY88 (Schultz et al., Gene 54:113-123 (1987)), pYES2 (Invitrogen Corporation, San Diego, Calif.), and picZ (Invitrogen Corp, San Diego, Calif.).

Alternatively, a polypeptide described herein can be expressed in insect cells using baculovirus expression vectors. Baculovirus vectors available for expression of proteins in cultured insect cells (e.g., Sf9 cells) include, for example, the pAc series (Smith et al., Mol. Cell Biol. 3:2156-2165 (1983)) and the pVL series (Lucklow et al., Virology 170:31-39 (1989)).

In yet another embodiment, the nucleic acids described herein can be expressed in mammalian cells using a mammalian expression vector. Examples of mammalian expression vectors include pCDM8 (Seed, Nature 329:840 (1987)) and pMT2PC (Kaufman et al., EMBO J. 6:187-195 (1987)). When used in mammalian cells, the expression vector's control functions can be provided by viral regulatory elements. For example, commonly used promoters are derived from polyoma, Adenovirus 2, cytomegalovirus, and Simian Virus 40. Other suitable expression systems for both prokaryotic and eukaryotic cells are described in chapters 16 and 17 of Sambrook et al., eds., Molecular Cloning: A Laboratory Manual. 2nd, ed., Cold Spring Harbor Laboratory, Cold Spring Harbor Laboratory Press, Cold Spring Harbor, N.Y. (1989).

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. supra.

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to antibiotics) can be introduced into the host cells along with the gene of interest. Selectable markers include those that confer resistance to drugs, such as ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

### Transport Proteins

Transport proteins can export polypeptides and organic compounds (e.g., fatty esters) out of a host cell. Many transport and efflux proteins serve to excrete a wide variety of compounds and can be naturally modified to be selective for particular types of fatty esters.

Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabditis elegans, Arabidopsis thalania, Alkaligenes eutrophus, and Rhodococcus erythropolis. Exemplary ABC transport proteins that can be used have been described in, for example, WO 08/119,082, the disclosure of which is incorporated herein by reference. Exemplary GenBank Accession numbers include, without limitation, CER5 [GenBank Accession No: At1g51500, AY734542, At3g21090, or At1g51460], AtMRP5 [GenBank Accession No: NP_{—}171908], AmiS2 [GenBank Accession No. JC5491], or AtPGP1 [GenBank Accession No. NP_{—}181228]. Host cells can also be chosen for their endogenous ability to secrete organic compounds. The efficiency of organic compound production and secretion into the host cell environment (e.g., culture medium, fermentation broth) can be expressed as a ratio of intracellular product to extracellular product. In some examples, the ratio can be about 5:1, 4:1, 3:1, 2:1, 1:1, 1:2, 1:3, 1:4, or 1:5.

In certain embodiments, the fatty esters produced by the host cells herein are present in the extracellular environment. In some embodiments, the fatty esters are isolated from the extracellular environment of the host cell. In some other embodiments, the fatty esters are spontaneously secreted, partially or completely, from the host cell. In further embodiments, the fatty esters are transported into the extracellular environment, optionally with the aid of one or more suitable transport proteins as described herein. In other embodiments, the fatty esters are passively transported into the extracellular environment.

### Fermentation

The production and isolation of fatty esters can be enhanced by employing beneficial fermentation techniques. One method for maximizing production while reducing costs is increasing the percentage of the carbon source that is converted to fatty ester products.

During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311:1113, 2006; Venturi FEMS Microbio. Rev. 30:274-291 (2006); and Reading et al., FEMS Microbiol. Lett. 254:1-11 (2006)) can be used to activate checkpoint genes, such as p53, p21, or other checkpoint genes.

Genes that can be activated to stop cell replication and growth in E. coli include umuDC genes. The overexpression of umuDC genes stops the progression from stationary phase to exponential growth (Murli et al., J. of Bact. 182:1127 (2000)). UmuC is a DNA polymerase that can carry out translesion synthesis over non-coding lesions—the mechanistic basis of most UV and chemical mutagenesis. The umuDC gene products are involved in the process of translesion synthesis and also serve as a DNA sequence damage checkpoint. The umuDC gene products include UmuC, UmuD, umuD′, UmuD′₂C, UmuD′₂, and UmuD₂. Simultaneously, product-producing genes can be activated, thus minimizing the need for replication and maintenance pathways to be used while a fatty ester is being made. Host cells can also be engineered to express umuC and umuD from E. coli in pBAD24 under the prpBCDE promoter system through de novo synthesis of this gene with the appropriate end-product production genes.

The percentage of input carbons converted to fatty esters can be a cost driver. The more efficient the process is (i.e., the higher the percentage of input carbons converted to fatty esters), the less expensive the process will be. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other organic compounds and carbon sources. Typical efficiencies in the literature are approximately less than 5%. Host cells engineered to produce fatty esters can have greater than about 1, 3, 5, 10, 15, 20, 25, and 30% efficiency. In one example, host cells can exhibit an efficiency of about 10% to about 25%. In other examples, such host cells can exhibit an efficiency of about 25% to about 30%. In other examples, host cells can exhibit greater than 30% efficiency.

The host cell can be additionally engineered to express recombinant cellulosomes, such as those described in PCT application number PCT/US2007/003736. These cellulosomes can allow the host cell to use cellulosic material as a carbon source. For example, the host cell can be additionally engineered to express invertases (EC 3.2.1.26) so that sucrose can be used as a carbon source. Similarly, the host cell can be engineered using the teachings described in U.S. Pat. Nos. 5,000,000; 5,028,539; 5,424,202; 5,482,846; and 5,602,030; so that the host cell can assimilate carbon efficiently and use cellulosic materials as carbon sources.

In one example, the fermentation chamber can enclose a fermentation that is undergoing a continuous reduction. In this instance, a stable reductive environment can be created. The electron balance can be maintained by the release of carbon dioxide (in gaseous form). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of fatty acids.

For small scale production, the engineered host cells can be grown in batches of, for example, about 100 mL, 500 mL, 1 L, 2 L, 5 L, or 10 L; fermented; and induced to express desired biosynthetic genes based on the specific genes encoded in the appropriate plasmids. For large scale production, the engineered host cells can be grown in batches of about 10 L, 100 L, 1000 L, 10,000 L, 100,000 L, 1,000,000 L or larger; fermented; and induced to express desired biosynthetic genes based on the specific genes encoded in the appropriate plasmids or incorporated into the host cell's genome.

For example, a suitable production host, such as E. coli cells, harboring plasmids containing the desired biosynthetic genes or having the biosynthetic genes integrated in its chromosome can be incubated in a suitable reactor, for example a 1 L reactor, for 20 h at 37° C. in M9 medium supplemented with 2% glucose, carbenicillin, and chloramphenicol. When the OD₆₀₀ of the culture reaches 0.9, the production host can be induced with IPTG to activate the engineered gene systems for fatty ester production. After incubation, the spent media can be extracted and the organic phase can be examined for the presence of fatty esters using GC-MS.

In some instances, after the first hour of induction, aliquots of no more than about 10% of the total cell volume can be removed each hour and allowed to sit without agitation to allow the fatty esters to rise to the surface and undergo a spontaneous phase separation or precipitation. The fatty ester component can then be collected, and the aqueous phase returned to the reaction chamber. The reaction chamber can be operated continuously. When the OD₆₀₀ drops below 0.6, the cells can be replaced with a new batch grown from a seed culture.

### Glucose

In some instances, the methods disclosed herein are performed using glucose as a carbon source. In certain instances, microorganisms are grown in a culture medium containing an initial glucose concentration of about 2 g/L to about 50 g/L, such as about 5 g/L to about 20 g/L. In some instances, the glucose concentration of the culture medium decreases from the initial glucose concentration as the microorganisms consume the glucose, and a concentration of about 0 g/L to about 5 g/L glucose is maintained in the culture medium during the fatty ester production process. In certain instances, glucose is fed to the microorganisms in a solution of about 50% to about 65% glucose.

In some instances, the feed rate of glucose is set to match the cells' growth rate to avoid excess accumulation of glucose (i.e., >0% glucose) in the fermentor. In other instances, and a low concentration of excess glucose (e.g., about 2 g/L to about 5 g/L) is maintained.

In certain instances, fatty esters can be produced from carbohydrates other than glucose, including but not limited to fructose, hydrolyzed sucrose, hydrolyzed molasses and glycerol.

### Post-Production Processing

The fatty esters produced during fermentation can be separated from the fermentation media. Any known technique for separating fatty esters from aqueous media can be used. One exemplary separation process is a two phase (bi-phasic) separation process. This process involves fermenting the genetically engineered host cells under conditions sufficient to produce a fatty ester, allowing the fatty ester to collect in an organic phase, and separating the organic phase from the aqueous fermentation broth. This method can be practiced in both a batch and continuous fermentation processes.

Bi-phasic separation uses the relative immiscibility of fatty esters to facilitate separation. Immiscible refers to the relative inability of a compound to dissolve in water and is defined by the compound's partition coefficient. One of ordinary skill in the art will appreciate that by choosing a fermentation broth and organic phase, such that the fatty ester being produced has a high logP value, the fatty ester can separate into the organic phase, even at very low concentrations, in the fermentation vessel.

The fatty esters produced by the methods described herein can be relatively immiscible in the fermentation broth, as well as in the cytoplasm. Therefore, the fatty ester can collect in an organic phase either intracellularly or extracellularly. The collection of the products in the organic phase can lessen the impact of the fatty ester on cellular function and can allow the host cell to produce more product.

The methods described herein can result in the production of homogeneous compounds wherein at least about 60%, 70%, 80%, 90%, or 95% of the fatty esters produced will have carbon chain lengths that vary by less than about 6 carbons, less than about 4 carbons, or less than about 2 carbons. These compounds can also be produced with a relatively uniform degree of saturation. These compounds can be used directly as fuels, fuel additives, starting materials for production of other chemical compounds (e.g., polymers, surfactants, plastics, textiles, solvents, adhesives, etc.), or personal care additives. These compounds can also be used as feedstock for subsequent reactions, for example, hydrogenation, catalytic cracking (e.g., via hydrogenation, pyrolisis, or both), to make other products.

In some embodiments, the fatty esters produced using methods described herein can contain between about 50% and about 90% carbon; or between about 5% and about 25% hydrogen. In other embodiments, the fatty esters produced using methods described herein can contain between about 65% and about 85% carbon; or between about 10% and about 15% hydrogen.

### Bioproducts

Bioproducts (e.g., the fatty esters produced in accordance with the present disclosure) comprising biologically produced organic compounds, and in particular, the fatty esters biologically produced using the fatty acid biosynthetic pathway herein, have not been produced from renewable sources and, as such, are new compositions of matter. These new bioproducts can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting (see, e.g., U.S. Pat. No. 7,169,588, which is herein incorporated by reference).

The ability to distinguish bioproducts from petroleum based organic compounds is beneficial in tracking these materials in commerce. For example, organic compounds or chemicals comprising both biologically based and petroleum based carbon isotope profiles may be distinguished from organic compounds and chemicals made only of petroleum based materials. Hence, the bioproducts herein can be followed or tracked in commerce on the basis of their unique carbon isotope profile.

Bioproducts can be distinguished from petroleum based organic compounds by comparing the stable carbon isotope ratio (¹³C/¹²C) in each fuel. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C₃ plants (the broadleaf), C₄ plants (the grasses), and marine carbonates all show significant differences in ¹³C/¹²C and the corresponding δ¹³C values. Furthermore, lipid matter of C₃ and C₄ plants analyze differently than materials derived from the carbohydrate components of the same plants as a consequence of the metabolic pathway.

Within the precision of measurement, ¹³C shows large variations due to isotopic fractionation effects, the most significant of which for bioproducts is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C₃” (or Calvin-Benson) photosynthetic cycle and those that incorporate the “C₄” (or Hatch-Slack) photosynthetic cycle.

In C₃ plants, the primary CO₂ fixation or carboxylation reaction involves the enzyme ribulose-1,5-diphosphate carboxylase, and the first stable product is a 3-carbon compound. C₃ plants, such as hardwoods and conifers, are dominant in the temperate climate zones.

In C₄ plants, an additional carboxylation reaction involving another enzyme, phosphoenol-pyruvate carboxylase, is the primary carboxylation reaction. The first stable carbon compound is a 4-carbon acid that is subsequently decarboxylated. The CO₂ thus released is refixed by the C₃ cycle. Examples of C₄ plants are tropical grasses, corn, and sugar cane.

Both C₄ and C₃ plants exhibit a range of ¹³C/¹²C isotopic ratios, but typical values are about −7 to about −13 per mil for C₄ plants and about −19 to about −27 per mil for C₃ plants (see, e.g., Stuiver et al., Radiocarbon 19:355 (1977)). Coal and petroleum fall generally in this latter range. The ¹³C measurement scale was originally defined by a zero set by Pee Dee Belemnite (PDB) limestone, where values are given in parts per thousand deviations from this material. The “δ¹³C” values are expressed in parts per thousand (per mil), abbreviated, ‰, and are calculated as follows:

δ¹³C(‰)=[(¹³C/¹²C)ₛₐₘₚₗₑ−(¹³C/¹²C)_{standard}]/(¹³C/¹²C)_{standard}×1000

Since the PDB reference material (RM) has been exhausted, a series of alternative RMs have been developed in cooperation with the IAEA, USGS, NIST, and other selected international isotope laboratories. Notations for the per mil deviations from PDB is δ¹³C. Measurements are made on CO₂ by high precision stable ratio mass spectrometry (IRMS) on molecular ions of masses 44, 45, and 46.

The compositions described herein include bioproducts produced by any of the methods described herein, including, for example, the fatty ester products. Specifically, the bioproduct can have a δ¹³C of about −28 or greater, about −27 or greater, −20 or greater, −18 or greater, −15 or greater, −13 or greater, −10 or greater, or −8 or greater. For example, the bioproduct can have a δ¹³C of about −30 to about −15, about −27 to about −19, about −25 to about −21, about −15 to about −5, about −13 to about −7, or about −13 to about −10. In other instances, the bioproduct can have a δ¹³C of about −10, −11, −12, or −12.3.

Bioproducts, including the bioproducts produced in accordance with the disclosure herein, can also be distinguished from petroleum based organic compounds by comparing the amount of ¹⁴C in each compound. Because ¹⁴C has a nuclear half life of 5730 years, petroleum based fuels containing “older” carbon can be distinguished from bioproducts which contain “newer” carbon (see, e.g., Currie, “Source Apportionment of Atmospheric Particles”, Characterization of Environmental Particles, J. Buffle and H. P. van Leeuwen, Eds., 1 of Vol. I of the IUPAC Environmental Analytical Chemistry Series (Lewis Publishers, Inc) 3-74, (1992)).

The basic assumption in radiocarbon dating is that the constancy of ¹⁴C concentration in the atmosphere leads to the constancy of ¹⁴C in living organisms. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of about 1.2×10⁻¹², with an approximate relaxation “half-life” of 7-10 years. (This latter half-life must not be taken literally; rather, one must use the detailed atmospheric nuclear input/decay function to trace the variation of atmospheric and biospheric ¹⁴C since the onset of the nuclear age.)

It is this latter biospheric ¹⁴C time characteristic that holds out the promise of annual dating of recent biospheric carbon. ¹⁴C can be measured by accelerator mass spectrometry (AMS), with results given in units of “fraction of modern carbon” (f_{M}). f_{M} is defined by National Institute of Standards and Technology (NIST) Standard Reference Materials (SRMs) 4990B and 4990C. As used herein, “fraction of modern carbon” or “f_{M}” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

The invention provides a bioproduct comprising one or more fatty esters, which can have an f_{M} ¹⁴C of at least about 1. For example, the bioproduct of the invention can have an f_{M} ¹⁴C of at least about 1.01, an f_{M} ¹⁴C of about 1 to about 1.5, an f_{M} ¹⁴C of about 1.04 to about 1.18, or an f_{M} ¹⁴C of about 1.111 to about 1.124.

Another measurement of ¹⁴C is known as the percent of modern carbon (pMC). For an archaeologist or geologist using ¹⁴C dates, AD 1950 equals “zero years old”. This also represents 100 pMC. “Bomb carbon” in the atmosphere reached almost twice the normal level in 1963 at the peak of thermo-nuclear weapons. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum based compounds will have a pMC value of zero. Combining fossil carbon with present day carbon will result in a dilution of the present day pMC content. By presuming 107.5 pMC represents the ¹⁴C content of present day biomass materials and 0 pMC represents the ¹⁴C content of petroleum based products, the measured pMC value for that material will reflect the proportions of the two component types. For example, a material derived 100% from present day soybeans would give a radiocarbon signature near 107.5 pMC. If that material was diluted 50% with petroleum based products, it would give a radiocarbon signature of approximately 54 pMC.

A biologically based carbon content is derived by assigning “100%” equal to 107.5 pMC and “0%” equal to 0 pMC. For example, a sample measuring 99 pMC will give an equivalent biologically based carbon content of 93%. This value is referred to as the mean biologically based carbon result and assumes all the components within the analyzed material originated either from present day biological material or petroleum based material.

A bioproduct comprising one or more fatty esters as described herein can have a pMC of at least about 50, 60, 70, 75, 80, 85, 90, 95, 96, 97, 98, 99, or 100. In other instances, a bioproduct described herein can have a pMC of between about 50 and about 100; about 60 and about 100; about 70 and about 100; about 80 and about 100; about 85 and about 100; about 87 and about 98; or about 90 and about 95. In yet other instances, a bioproduct described herein can have a pMC of about 90, 91, 92, 93, 94, or 94.2.

Fatty esters produced by the methods described herein can be used as biofuels. For example, any fatty acid methyl ester described herein can be used solely or as a component of biodiesel.

The invention is further illustrated by the following examples. The examples are provided for illustrative purposes only. They are not to be construed as limiting the scope or content of the invention in any way.

### EXAMPLES

### Example 1

### Production of E. coli MG1655 ΔfadE

This example describes the construction of a genetically engineered microorganism wherein the expression of a fatty acid degradation enzyme is attenuated.

The fadE gene of E. coli MG1655 (an E. coli K strain) was deleted using the Lambda Red (also known as the Red-Driven Integration) system described in Datsenko et al., Proc. Natl. Acad. Sci. USA 97: 6640-6645 (2000), with the following modifications.

Two primers were used to create the deletion:

Del-fadE-F

(SEQ ID NO: 1)

5′-AAAAACAGCAACAATGTGAGCTTTGTTGTAATTATATTGTAAACATA
TTGATTCCGGGGATCCGTCGACC
Del-fadE-R

(SEQ ID NO: 2)

5′-AAACGGAGCCTTTCGGCTCCGTTATTCATTTACGCGGCTTCAACTTT
CCTGTAGGCTGGAGCTGCTTC

The Del-fadE-F and Del-fadE-R primers were used to amplify the Kanamycin resistance (Km^{R}) cassette from plasmid pKD13 (as described in Datsenko et al., supra) by PCR. The PCR product was then used to transform electrocompetent E. coli MG1655 cells containing pKD46 (described in Datsenko et al., supra). These cells had been previously induced with arabinose for 3-4 h. Following a 3-h outgrowth in a super optimal broth with catabolite repression (SOC) medium at 37° C., the cells were plated on Luria agar plates containing 50 μg/mL Kanamycin. Resistant colonies were identified and isolated after an overnight incubation at 37° C. Disruption of the fadE gene was confirmed in select colonies using PCR amplification with primers fadE-L2 and fadE-R1, which were designed to flank the fadE gene:

| | |
|---|---|
| fadE-L2 5′-CGGGCAGGTGCTATGACCAGGAC | (SEQ ID NO: 3) |
| fadE-R1 5′-CGCGGCGTTGACCGGCAGCCTGG | (SEQ ID NO: 4) |

After the fadE deletion was confirmed, a single colony was used to remove the Km^{R} marker, using the pCP20 plasmid as described in Datsenko et al., supra. The resulting MG1655 E. coli strain with the fadE gene deleted and the Km^{R} marker removed was named E. coli MG1655 ΔfadE, or E. coli MG1655 D1.

### Example 2

### Production of E. coli MG1655 ΔfadE ΔfhuA

This example describes the construction of a genetically engineered microorganism in which the expression of a fatty acid degradation enzyme and an outer membrane protein receptor are attenuated.

The fhuA (also known as tonA) gene of E. coli MG1655, which encodes a ferrichrome outer membrane transporter (GenBank Accession No. NP_{—}414692), was deleted from strain E. coli MG1655 D1 of Example 1 using the Lambda Red system described in Datsenko et al., supra, but with the following modifications.

Two primers were used to create the deletion:

Del-fhuA-F

(SEQ ID NO: 5)

5′-ATCATTCTCGTTTACGTTATCATTCACTTTACATCAGAGATATACCA
ATGATTCCGGGGATCCGTCGACC;
Del-fhuA-R

(SEQ ID NO: 6)

5′-GCACGGAAATCCGTGCCCCAAAAGAGAAATTAGAAACGGAAGGTTGC
GG TTGTAGGCTGGAGCTGCTTC

The Del-fhuA-F and Del-fhuA-R primers were used to amplify the Km^{R} cassette from plasmid pKD13 by PCR. The PCR product obtained was used to transform the electrocompetent E. coli MG1655 D1 cells containing pKD46 (see Example 1). These cells had been previously induced with arabinose for 3-4 h. Following a 3-h outgrowth in SOC medium at 37° C., the cells were plated on Luria agar plates containing 50 μg/mL Kanamycin. Kanamycin resistant colonies were identified and isolated after an overnight incubation at 37° C. Disruption of the fhuA gene was confirmed in select colonies by PCR amplification with primers fhuA-verF and fhuA-verR, which were designed to flank the fhuA gene.

Confirmation of the deletion was performed using the following primers:

| | |
|---|---|
| fhuA-verF 5′-CAACAGCAACCTGCTCAGCAA | (SEQ ID NO: 7) |
| fhuA-verR 5′-AAGCTGGAGCAGCAAAGCGTT | (SEQ ID NO: 8) |

After the fhuA deletion was confirmed, a single colony was used to remove the Km^{R} marker, using the pCP20 plasmid as described in Datsenko et al., supra. The resulting MG1655 E. coli strain having the fadE and fhuA gene deletions was named E. coli MG1655 ΔfadE ΔfhuA, or E. coli MG1655 DV2.

### Example 3

### Production of E. coli MG1655 ΔfadE, ΔfhuA, lacZ:: 'tesA fadD atfA1

This example describes the construction of a genetically engineered microorganism in which nucleotide sequences encoding a thioesterase, an acyl-CoA synthase, and an ester synthase are integrated into the microorganism's chromosome.

The following nucleotide sequences, 'tesA, fadD, and aftA1, were integrated into the chromosome of E. coli MG1655 ΔfadE ΔfhuA strain (or DV2 strain, see Example 2) at the lacZ locus. The sequences were integrated in the order of 'tesA, followed by fadD, and followed by aftA1.

'tesA is a nucleotide sequence comprising a leaderless E. coli tesA (GenBank entry AAC73596, refseq accession U00096.2). 'tesA encodes an E. coli thioesterase (EC 3.1.1.5, 3.1.2.-) in which the first twenty-five amino acids were deleted and the amino acid in position 26, alanine, was replaced with methionine. That methionine then became the first amino acid of 'tesA. See Cho et al., J. Biol. Chem., 270:4216-4219 (1995).

E. coli fadD (GenBank entry AAC74875; REFSEQ: accession U00096.2) encodes an acyl-CoA synthase.

Alcanivorax borkumensis strain SK2 atfA1 (GenBank entry YP_{—}694462; REFSEQ: accession NC_{—}008260.1) encodes an ester synthase.

'tesA, fadD, and atfA1 were integrated into the chromosome of E. coli MG1655 DV2 at the lacZ locus, all under the control of a Trc promoter, as described below.

Design and Creation of a 'tesA, fadD, atfA1 Integration Cassette
Construction of the 'tesA Plasmid

'tesA was amplified from a pETDuet-1-'tesA plasmid constructed as described below. (see also, e.g., WO 2007/136762 A2, which is incorporated by reference). The 'tesA gene was cloned into an NdeI/AvrII digested pETDuet-1 plasmid (Novagen, Madison, Wis.).

Construction of the fadD Plasmid

fadD was amplified from a pHZ1.61 plasmid constructed as described below. A fadD gene was cloned into a pCDFDuet-1 plasmid (Novagen, Madison, Wis.) under the control of a T7 promoter, generating a pHZ1.61 plasmid containing the following nucleotide sequence:

(SEQ ID NO: 9)

GGGGAATTGTGAGCGGATAACAATTCCCCTGTAGAAATAATTTTGTTTAA
CTTTAATAAGGAGATATACCATGGTGAAGAAGGTTTGGCTTAACCGTTAT
CCCGCGGACGTTCCGACGGAGATCAACCCTGACCGTTATCAATCTCTGGT
AGATATGTTTGAGCAGTCGGTCGCGCGCTACGCCGATCAACCTGCGTTTG
TGAATATGGGGGAGGTAATGACCTTCCGCAAGCTGGAAGAACGCAGTCGC
GCGTTTGCCGCTTATTTGCAACAAGGGTTGGGGCTGAAGAAAGGCGATCG
CGTTGCGTTGATGATGCCTAATTTATTGCAATATCCGGTGGCGCTGTTTG
GCATTTTGCGTGCCGGGATGATCGTCGTAAACGTTAACCCGTTGTATACC
CCGCGTGAGCTTGAGCATCAGCTTAACGATAGCGGCGCATCGGCGATTGT
TATCGTGTCTAACTTTGCTCACACACTGGAAAAAGTGGTTGATAAAACCG
CCGTTCAGCACGTAATTCTGACCCGTATGGGCGATCAGCTATCTACGGCA
AAAGGCACGGTAGTCAATTTCGTTGTTAAATACATCAAGCGTTTGGTGCC
GAAATACCATCTGCCAGATGCCATTTCATTTCGTAGCGCACTGCATAACG
GCTACCGGATGCAGTACGTCAAACCCGAACTGGTGCCGGAAGATTTAGCT
TTTCTGCAATACACCGGCGGCACCACTGGTGTGGCGAAAGGCGCGATGCT
GACTCACCGCAATATGCTGGCGAACCTGGAACAGGTTAACGCGACCTATG
GTCCGCTGTTGCATCCGGGCAAAGAGCTGGTGGTGACGGCGCTGCCGCTG
TATCACATTTTTGCCCTGACCATTAACTGCCTGCTGTTTATCGAACTGGG
TGGGCAGAACCTGCTTATCACTAACCCGCGCGATATTCCAGGGTTGGTAA
AAGAGTTAGCGAAATATCCGTTTACCGCTATCACGGGCGTTAACACCTTG
TTCAATGCGTTGCTGAACAATAAAGAGTTCCAGCAGCTGGATTTCTCCAG
TCTGCATCTTTCCGCAGGCGGAGGGATGCCAGTGCAGCAAGTGGTGGCAG
AGCGTTGGGTGAAACTGACAGGACAGTATCTGCTGGAAGGCTATGGCCTT
ACCGAGTGTGCGCCGCTGGTCAGCGTTAACCCATATGATATTGATTATCA
TAGTGGTAGCATCGGTTTGCCGGTGCCGTCGACGGAAGCCAAACTGGTGG
ATGATGATGATAATGAAGTACCACCGGGTCAACCGGGTGAGCTTTGTGTC
AAAGGACCGCAGGTGATGCTGGGTTACTGGCAGCGTCCGGATGCTACAGA
TGAGATCATCAAAAATGGCTGGTTACACACCGGCGACATCGCGGTGATGG
ATGAAGAAGGGTTCCTGCGCATTGTCGATCGTAAAAAAGACATGATTCTG
GTTTCCGGTTTTAACGTCTATCCCAACGAGATTGAAGATGTCGTCATGCA
GCATCCTGGCGTACAGGAAGTCGCGGCTGTTGGCGTACCTTCCGGCTCCA
GTGGTGAAGCGGTGAAAATCTTCGTAGTGAAAAAAGATCCATCGCTTACC
GAAGAGTCACTGGTGACCTTTTGCCGCCGTCAGCTCACGGGCTACAAAGT
ACCGAAGCTGGTGGAGTTTCGTGATGAGTTACCGAAATCTAACGTCGGAA
AAATTTTGCGACGAGAATTACGTGACGAAGCGCGCGGCAAAGTGGACAAT
AAAGCCTGAAAGCTTGCGGCCGCATAATGCTTAAGTCGAACAGAAAGTAA
TCGTATTGTACACGGCCGCATAATCGAAATTAATACGACTCACTATAGGG
GAATTGTGAGCGGATAACAATTCCCCATCTTAGTATATTAGTTAAGTATA
AGAAGGAGATATACATATGCGCCCATTACATCCGATTGATTTTATATTCC
TGTCACTAGAAAAAAGACAACAGCCTATGCATGTAGGTGGTTTATTTTTG
TTTCAGATTCCTGATAACGCCCCAGACACCTTTATTCAAGATCTGGTGAA
TGATATCCGGATATCAAAATCAATCCCTGTTCCACCATTCAACAATAAAC
TGAATGGGCTTTTTTGGGATGAAGATGAAGAGTTTGATTTAGATCATCAT
TTTCGTCATATTGCACTGCCTCATCCTGGTCGTATTCGTGAATTGCTTAT
TTATATTTCACAAGAGCACAGTACGCTGCTAGATCGGGCAAAGCCCTTGT
GGACCTGCAATATTATTGAAGGAATTGAAGGCAATCGTTTTGCCATGTAC
TTCAAAATTCACCATGCGATGGTCGATGGCGTTGCTGGTATGCGGTTAAT
TGAAAAATCACTCTCCCATGATGTAACAGAAAAAAGTATCGTGCCACCTT
GGTGTGTTGAGGGAAAACGTGCAAAGCGCTTAAGAGAACCTAAAACAGGT
AAAATTAAGAAAATCATGTCTGGTATTAAGAGTCAGCTTCAGGCGACACC
CACAGTCATTCAAGAGCTTTCTCAGACAGTATTTAAAGATATTGGACGTA
ATCCTGATCATGTTTCAAGCTTTCAGGCGCCTTGTTCTATTTTGAATCAG
CGTGTGAGCTCATCGCGACGTTTTGCAGCACAGTCTTTTGACCTAGATCG
TTTTCGTAATATTGCCAAATCGTTGAATGTGACCATTAATGATGTTGTAC
TAGCGGTATGTTCTGGTGCATTACGTGCGTATTTGATGAGTCATAATAGT
TTGCCTTCAAAACCATTAATTGCCATGGTTCCAGCCTCTATTCGCAATGA
CGATTCAGATGTCAGCAACCGTATTACGATGATTCTGGCAAATTTGGCAA
CCCACAAAGATGATCCTTTACAACGTCTTGAAATTATCCGCCGTAGTGTT
CAAAACTCAAAGCAACGCTTCAAACGTATGACCAGCGATCAGATTCTAAA
TTATAGTGCTGTCGTATATGGCCCTGCAGGACTCAACATAATTTCTGGCA
TGATGCCAAAACGCCAAGCCTTCAATCTGGTTATTTCCAATGTGCCTGGC
CCAAGAGAGCCACTTTACTGGAATGGTGCCAAACTTGATGCACTCTACCC
AGCTTCAATTGTATTAGACGGTCAAGCATTGAATATTACAATGACCAGTT
ATTTAGATAAACTTGAAGTTGGTTTGATTGCATGCCGTAATGCATTGCCA
AGAATGCAGAATTTACTGACACATTTAGAAGAAGAAATTCAACTATTTGA
AGGCGTAATTGCAAAGCAGGAAGATATTAAAACAGCCAATTAAAAACAAT
AAACTTGATTTTTTAATTTATCAGATAAAACTAAAGGGCTAAATTAGCCC
TCCTAGGCTGCTGCCACCGCTGAGCAATAACTAGCATAACCCCTTGGGGC
CTCTAAACGGGTCTTGAGGGGTTTTTTGCTGAAACCTCAGGCATTTGAGA
AGCACACGGTCACACTGCTTCCGGTAGTCAATAAACCGGTAAACCAGCAA
TAGACATAAGCGGCTATTTAACGACCCTGCCCTGAACCGACGACCGGGTC
ATCGTGGCCGGATCTTGCGGCCCCTCGGCTTGAACGAATTGTTAGACATT
ATTTGCCGACTACCTTGGTGATCTCGCCTTTCACGTAGTGGACAAATTCT
TCCAACTGATCTGCGCGCGAGGCCAAGCGATCTTCTTCTTGTCCAAGATA
AGCCTGTCTAGCTTCAAGTATGACGGGCTGATACTGGGCCGGCAGGCGCT
CCATTGCCCAGTCGGCAGCGACATCCTTCGGCGCGATTTTGCCGGTTACT
GCGCTGTACCAAATGCGGGACAACGTAAGCACTACATTTCGCTCATCGCC
AGCCCAGTCGGGCGGCGAGTTCCATAGCGTTAAGGTTTCATTTAGCGCCT
CAAATAGATCCTGTTCAGGAACCGGATCAAAGAGTTCCTCCGCCGCTGGA
CCTACCAAGGCAACGCTATGTTCTCTTGCTTTTGTCAGCAAGATAGCCAG
ATCAATGTCGATCGTGGCTGGCTCGAAGATACCTGCAAGAATGTCATTGC
GCTGCCATTCTCCAAATTGCAGTTCGCGCTTAGCTGGATAACGCCACGGA
ATGATGTCGTCGTGCACAACAATGGTGACTTCTACAGCGCGGAGAATCTC
GCTCTCTCCAGGGGAAGCCGAAGTTTCCAAAAGGTCGTTGATCAAAGCTC
GCCGCGTTGTTTCATCAAGCCTTACGGTCACCGTAACCAGCAAATCAATA
TCACTGTGTGGCTTCAGGCCGCCATCCACTGCGGAGCCGTACAAATGTAC
GGCCAGCAACGTCGGTTCGAGATGGCGCTCGATGACGCCAACTACCTCTG
ATAGTTGAGTCGATACTTCGGCGATCACCGCTTCCCTCATACTCTTCCTT
TTTCAATATTATTGAAGCATTTATCAGGGTTATTGTCTCATGAGCGGATA
CATATTTGAATGTATTTAGAAAAATAAACAAATAGCTAGCTCACTCGGTC
GCTACGCTCCGGGCGTGAGACTGCGGCGGGCGCTGCGGACACATACAAAG
TTACCCACAGATTCCGTGGATAAGCAGGGGACTAACATGTGAGGCAAAAC
AGCAGGGCCGCGCCGGTGGCGTTTTTCCATAGGCTCCGCCCTCCTGCCAG
AGTTCACATAAACAGACGCTTTTCCGGTGCATCTGTGGGAGCCGTGAGGC
TCAACCATGAATCTGACAGTACGGGCGAAACCCGACAGGACTTAAAGATC
CCCACCGTTTCCGGCGGGTCGCTCCCTCTTGCGCTCTCCTGTTCCGACCC
TGCCGTTTACCGGATACCTGTTCCGCCTTTCTCCCTTACGGGAAGTGTGG
CGCTTTCTCATAGCTCACACACTGGTATCTCGGCTCGGTGTAGGTCGTTC
GCTCCAAGCTGGGCTGTAAGCAAGAACTCCCCGTTCAGCCCGACTGCTGC
GCCTTATCCGGTAACTGTTCACTTGAGTCCAACCCGGAAAAGCACGGTAA
AACGCCACTGGCAGCAGCCATTGGTAACTGGGAGTTCGCAGAGGATTTGT
TTAGCTAAACACGCGGTTGCTCTTGAAGTGTGCGCCAAAGTCCGGCTACA
CTGGAAGGACAGATTTGGTTGCTGTGCTCTGCGAAAGCCAGTTACCACGG
TTAAGCAGTTCCCCAACTGACTTAACCTTCGATCAAACCACCTCCCCAGG
TGGTTTTTTCGTTTACAGGGCAAAAGATTACGCGCAGAAAAAAAGGATCT
CAAGAAGATCCTTTGATCTTTTCTACTGAACCGCTCTAGATTTCAGTGCA
ATTTATCTCTTCAAATGTAGCACCTGAAGTCAGCCCCATACGATATAAGT
TGTAATTCTCATGTTAGTCATGCCCCGCGCCCACCGGAAGGAGCTGACTG
GGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGG
AAACCTGTCGTGCCAGCTGCATTAATGAATCGGCCAACGCGCGGGGAGAG
GCGGTTTGCGTATTGGGCGCCAGGGTGGTTTTTCTTTTCACCAGTGAGAC
GGGCAACAGCTGATTGCCCTTCACCGCCTGGCCCTGAGAGAGTTGCAGCA
AGCGGTCCACGCTGGTTTGCCCCAGCAGGCGAAAATCCTGTTTGATGGTG
GTTAACGGCGGGATATAACATGAGCTGTCTTCGGTATCGTCGTATCCCAC
TACCGAGATGTCCGCACCAACGCGCAGCCCGGACTCGGTAATGGCGCGCA
TTGCGCCCAGCGCCATCTGATCGTTGGCAACCAGCATCGCAGTGGGAACG
ATGCCCTCATTCAGCATTTGCATGGTTTGTTGAAAACCGGACATGGCACT
CCAGTCGCCTTCCCGTTCCGCTATCGGCTGAATTTGATTGCGAGTGAGAT
ATTTATGCCAGCCAGCCAGACGCAGACGCGCCGAGACAGAACTTAATGGG
CCCGCTAACAGCGCGATTTGCTGGTGACCCAATGCGACCAGATGCTCCAC
GCCCAGTCGCGTACCGTCTTCATGGGAGAAAATAATACTGTTGATGGGTG
TCTGGTCAGAGACATCAAGAAATAACGCCGGAACATTAGTGCAGGCAGCT
TCCACAGCAATGGCATCCTGGTCATCCAGCGGATAGTTAATGATCAGCCC
ACTGACGCGTTGCGCGAGAAGATTGTGCACCGCCGCTTTACAGGCTTCGA
CGCCGCTTCGTTCTACCATCGACACCACCACGCTGGCACCCAGTTGATCG
GCGCGAGATTTAATCGCCGCGACAATTTGCGACGGCGCGTGCAGGGCCAG
ACTGGAGGTGGCAACGCCAATCAGCAACGACTGTTTGCCCGCCAGTTGTT
GTGCCACGCGGTTGGGAATGTAATTCAGCTCCGCCATCGCCGCTTCCACT
TTTTCCCGCGTTTTCGCAGAAACGTGGCTGGCCTGGTTCACCACGCGGGA
AACGGTCTGATAAGAGACACCGGCATACTCTGCGACATCGTATAACGTTA
CTGGTTTCACATTCACCACCCTGAATTGACTCTCTTCCGGGCGCTATCAT
GCCATACCGCGAAAGGTTTTGCGCCATTCGATGGTGTCCGGGATCTCGAC
GCTCTCCCTTATGCGACTCCTGCATTAGGAAATTAATACGACTCACTATA

Construction of the atfA1 Plasmid

atfA1 was amplified from a pHZ1.97-atfA1 plasmid constructed as described below. The atfA1 gene was synthesized by DNA2.0 (Menlo Park, Calif.) and cloned into an NdeI and AvrII digested pCOLA-Duet-1 plasmid (Novagen, Madison, Wis.), generating a pHZ1.97-atfA1 plasmid having the following nucleotide sequence:

(SEQ ID NO: 10)

GGGGAATTGTGAGCGGATAACAATTCCCCTGTAGAAATAATTTTGTTTAA
CTTTAATAAGGAGATATACCATGGGCAGCAGCCATCACCATCATCACCAC
AGCCAGGATCCGAATTCGAGCTCGGCGCGCCTGCAGGTCGACAAGCTTGC
GGCCGCATAATGCTTAAGTCGAACAGAAAGTAATCGTATTGTACACGGCC
GCATAATCGAAATTAATACGACTCACTATAGGGGAATTGTGAGCGGATAA
CAATTCCCCATCTTAGTATATTAGTTAAGTATAAGAAGGAGATATACATA
TGAAAGCGCTTAGCCCAGTGGATCAACTGTTCCTGTGGCTGGAAAAACGA
CAGCAACCCATGCACGTAGGCGGTTTGCAGCTGTTTTCCTTCCCGGAAGG
TGCCGGCCCCAAGTATGTGAGTGAGCTGGCCCAGCAAATGCGGGATTACT
GCCACCCAGTGGCGCCATTCAACCAGCGCCTGACCCGTCGACTCGGCCAG
TATTACTGGACTAGAGACAAACAGTTCGATATCGACCACCACTTCCGCCA
CGAAGCACTCCCCAAACCCGGTCGCATTCGCGAACTGCTTTCTTTGGTCT
CCGCCGAACATTCCAACCTGCTGGACCGGGAGCGCCCCATGTGGGAAGCC
CATTTGATCGAAGGGATCCGCGGTCGCCAGTTCGCTCTCTATTATAAGAT
CCACCATTCGGTGATGGATGGCATATCCGCCATGCGTATCGCCTCCAAAA
CGCTTTCCACTGACCCCAGTGAACGTGAAATGGCTCCGGCTTGGGCGTTC
AACACCAAAAAACGCTCCCGCTCACTGCCCAGCAACCCGGTTGACATGGC
CTCCAGCATGGCGCGCCTAACCGCGAGCATAAGCAAACAAGCTGCCACAG
TGCCCGGTCTCGCGCGGGAGGTTTACAAAGTCACCCAAAAAGCCAAAAAA
GATGAAAACTATGTGTCTATTTTTCAGGCTCCCGACACGATTCTGAATAA
TACCATCACCGGTTCACGCCGCTTTGCCGCCCAGAGCTTTCCATTACCGC
GCCTGAAAGTTATCGCCAAGGCCTATAACTGCACCATTAACACCGTGGTG
CTCTCCATGTGTGGCCACGCTCTGCGCGAATACTTGATTAGCCAACACGC
GCTGCCCGATGAGCCACTGATTGCAATGGTGCCCATGAGCCTGCGGCAGG
ACGACAGCACTGGCGGCAACCAGATCGGTATGATCTTGGCTAACCTGGGC
ACCCACATCTGTGATCCAGCTAATCGCCTGCGCGTCATCCACGATTCCGT
CGAGGAAGCCAAATCCCGCTTCTCGCAGATGAGCCCGGAAGAAATTCTCA
ATTTCACCGCCCTCACTATGGCTCCCACCGGCTTGAACTTACTGACCGGC
CTAGCGCCAAAATGGCGGGCCTTCAACGTGGTGATTTCCAACATACCCGG
GCCGAAAGAGCCGCTGTACTGGAATGGTGCACAGCTGCAAGGAGTGTATC
CAGTATCCATTGCCTTGGATCGCATCGCCCTAAATATCACCCTCACCAGT
TATGTAGACCAGATGGAATTTGGGCTTATCGCCTGCCGCCGTACTCTGCC
TTCCATGCAGCGACTACTGGATTACCTGGAACAGTCCATCCGCGAATTGG
AAATCGGTGCAGGAATTAAATAGTAACCTAGGCTGCTGCCACCGCTGAGC
AATAACTAGCATAACCCCTTGGGGCCTCTAAACGGGTCTTGAGGGGTTTT
TTGCTGAAACCTCAGGCATTTGAGAAGCACACGGTCACACTGCTTCCGGT
AGTCAATAAACCGGTAAACCAGCAATAGACATAAGCGGCTATTTAACGAC
CCTGCCCTGAACCGACGACAAGCTGACGACCGGGTCTCCGCAAGTGGCAC
TTTTCGGGGAAATGTGCGCGGAACCCCTATTTGTTTATTTTTCTAAATAC
ATTCAAATATGTATCCGCTCATGAATTAATTCTTAGAAAAACTCATCGAG
CATCAAATGAAACTGCAATTTATTCATATCAGGATTATCAATACCATATT
TTTGAAAAAGCCGTTTCTGTAATGAAGGAGAAAACTCACCGAGGCAGTTC
CATAGGATGGCAAGATCCTGGTATCGGTCTGCGATTCCGACTCGTCCAAC
ATCAATACAACCTATTAATTTCCCCTCGTCAAAAATAAGGTTATCAAGTG
AGAAATCACCATGAGTGACGACTGAATCCGGTGAGAATGGCAAAAGTTTA
TGCATTTCTTTCCAGACTTGTTCAACAGGCCAGCCATTACGCTCGTCATC
AAAATCACTCGCATCAACCAAACCGTTATTCATTCGTGATTGCGCCTGAG
CGAGACGAAATACGCGGTCGCTGTTAAAAGGACAATTACAAACAGGAATC
GAATGCAACCGGCGCAGGAACACTGCCAGCGCATCAACAATATTTTCACC
TGAATCAGGATATTCTTCTAATACCTGGAATGCTGTTTTCCCGGGGATCG
CAGTGGTGAGTAACCATGCATCATCAGGAGTACGGATAAAATGCTTGATG
GTCGGAAGAGGCATAAATTCCGTCAGCCAGTTTAGTCTGACCATCTCATC
TGTAACATCATTGGCAACGCTACCTTTGCCATGTTTCAGAAACAACTCTG
GCGCATCGGGCTTCCCATACAATCGATAGATTGTCGCACCTGATTGCCCG
ACATTATCGCGAGCCCATTTATACCCATATAAATCAGCATCCATGTTGGA
ATTTAATCGCGGCCTAGAGCAAGACGTTTCCCGTTGAATATGGCTCATAC
TCTTCCTTTTTCAATATTATTGAAGCATTTATCAGGGTTATTGTCTCATG
AGCGGATACATATTTGAATGTATTTAGAAAAATAAACAAATAGGCATGCT
AGCGCAGAAACGTCCTAGAAGATGCCAGGAGGATACTTAGCAGAGAGACA
ATAAGGCCGGAGCGAAGCCGTTTTTCCATAGGCTCCGCCCCCCTGACGAA
CATCACGAAATCTGACGCTCAAATCAGTGGTGGCGAAACCCGACAGGACT
ATAAAGATACCAGGCGTTTCCCCCTGATGGCTCCCTCTTGCGCTCTCCTG
TTCCCGTCCTGCGGCGTCCGTGTTGTGGTGGAGGCTTTACCCAAATCACC
ACGTCCCGTTCCGTGTAGACAGTTCGCTCCAAGCTGGGCTGTGTGCAAGA
ACCCCCCGTTCAGCCCGACTGCTGCGCCTTATCCGGTAACTATCATCTTG
AGTCCAACCCGGAAAGACACGACAAAACGCCACTGGCAGCAGCCATTGGT
AACTGAGAATTAGTGGATTTAGATATCGAGAGTCTTGAAGTGGTGGCCTA
ACAGAGGCTACACTGAAAGGACAGTATTTGGTATCTGCGCTCCACTAAAG
CCAGTTACCAGGTTAAGCAGTTCCCCAACTGACTTAACCTTCGATCAAAC
CGCCTCCCCAGGCGGTTTTTTCGTTTACAGAGCAGGAGATTACGACGATC
GTAAAAGGATCTCAAGAAGATCCTTTACGGATTCCCGACACCATCACTCT
AGATTTCAGTGCAATTTATCTCTTCAAATGTAGCACCTGAAGTCAGCCCC
ATACGATATAAGTTGTAATTCTCATGTTAGTCATGCCCCGCGCCCACCGG
AAGGAGCTGACTGGGTTGAAGGCTCTCAAGGGCATCGGTCGAGATCCCGG
TGCCTAATGAGTGAGCTAACTTACATTAATTGCGTTGCGCTCACTGCCCG
CTTTCCAGTCGGGAAACCTGTCGTGCCAGCTGCATTAATGAATCGGCCAA
CGCGCGGGGAGAGGCGGTTTGCGTATTGGGCGCCAGGGTGGTTTTTCTTT
TCACCAGTGAGACGGGCAACAGCTGATTGCCCTTCACCGCCTGGCCCTGA
GAGAGTTGCAGCAAGCGGTCCACGCTGGTTTGCCCCAGCAGGCGAAAATC
CTGTTTGATGGTGGTTAACGGCGGGATATAACATGAGCTGTCTTCGGTAT
CGTCGTATCCCACTACCGAGATGTCCGCACCAACGCGCAGCCCGGACTCG
GTAATGGCGCGCATTGCGCCCAGCGCCATCTGATCGTTGGCAACCAGCAT
CGCAGTGGGAACGATGCCCTCATTCAGCATTTGCATGGTTTGTTGAAAAC
CGGACATGGCACTCCAGTCGCCTTCCCGTTCCGCTATCGGCTGAATTTGA
TTGCGAGTGAGATATTTATGCCAGCCAGCCAGACGCAGACGCGCCGAGAC
AGAACTTAATGGGCCCGCTAACAGCGCGATTTGCTGGTGACCCAATGCGA
CCAGATGCTCCACGCCCAGTCGCGTACCGTCTTCATGGGAGAAAATAATA
CTGTTGATGGGTGTCTGGTCAGAGACATCAAGAAATAACGCCGGAACATT
AGTGCAGGCAGCTTCCACAGCAATGGCATCCTGGTCATCCAGCGGATAGT
TAATGATCAGCCCACTGACGCGTTGCGCGAGAAGATTGTGCACCGCCGCT
TTACAGGCTTCGACGCCGCTTCGTTCTACCATCGACACCACCACGCTGGC
ACCCAGTTGATCGGCGCGAGATTTAATCGCCGCGACAATTTGCGACGGCG
CGTGCAGGGCCAGACTGGAGGTGGCAACGCCAATCAGCAACGACTGTTTG
CCCGCCAGTTGTTGTGCCACGCGGTTGGGAATGTAATTCAGCTCCGCCAT
CGCCGCTTCCACTTTTTCCCGCGTTTTCGCAGAAACGTGGCTGGCCTGGT
TCACCACGCGGGAAACGGTCTGATAAGAGACACCGGCATACTCTGCGACA
TCGTATAACGTTACTGGTTTCACATTCACCACCCTGAATTGACTCTCTTC
CGGGCGCTATCATGCCATACCGCGAAAGGTTTTGCGCCATTCGATGGTGT
CCGGGATCTCGACGCTCTCCCTTATGCGACTCCTGCATTAGGAAATTAAT
ACGACTCACTATA

Construction of pACYC-PTrc Plasmid Containing 'tesA, fadD, and atfA1

A pACYC-PTrc vector having the following sequence was used to construct a pACYC-PTrc-'tesA-fadD-atfA1 plasmid. The nucleotide sequence of the pACYC-PTrc vector is as follows:

(SEQ ID NO: 11)

ACTCACCAGTCACAGAAAAGCATCTTACGGATGGCATGACAGTAAGAGAA
TTATGCAGTGCTGCCATAACCATGAGTGATAACACTGCGGCCAACTTACT
TCTGACAACGATCGGAGGACCGAAGGAGCTAACCGCTTTTTTGCACAACA
TGGGGGATCATGTAACTCGCCTTGATCGTTGGGAACCGGAGCTGAATGAA
GCCATACCAAACGACGAGCGTGACACCACGATGCCTGCAGCAATGGCAAC
AACGTTGCGCAAACTATTAACTGGCGAACTACTTACTCTAGCTTCCCGGC
AACAATTAATAGACTGGATGGAGGCGGATAAAGTTGCAGGACCACTTCTG
CGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGC
CCTCCCGTATCGTAGTTATCTACACGACGGGGAGTCAGGCAACTATGGAT
GAACGAAATAGACAGATCGCTGAGATAGGTGCCTCACTGATTAAGCATTG
GTAACTGTCAGACCAAGTTTACTCATATATACTTTAGATTGATTTAAAAC
TTCATTTTTAATTTAAAAGGATCTAGGTGAAGATCCTTTTTGATAATCTC
ATGACCAAAATCCCTTAACGTGAGTTTTCGTTCCACTGAGCGTCAGACCC
CTTAATAAGATGATCTTCTTGAGATCGTTTTGGTCTGCGCGTAATCTCTT
GCTCTGAAAACGAAAAAACCGCCTTGCAGGGCGGTTTTTCGAAGGTTCTC
TGAGCTACCAACTCTTTGAACCGAGGTAACTGGCTTGGAGGAGCGCAGTC
ACCAAAACTTGTCCTTTCAGTTTAGCCTTAACCGGCGCATGACTTCAAGA
CTAACTCCTCTAAATCAATTACCAGTGGCTGCTGCCAGTGGTGCTTTTGC
ATGTCTTTCCGGGTTGGACTCAAGACGATAGTTACCGGATAAGGCGCAGC
GGTCGGACTGAACGGGGGGTTCGTGCATACAGTCCAGCTTGGAGCGAACT
GCCTACCCGGAACTGAGTGTCAGGCGTGGAATGAGACAAACGCGGCCATA
ACAGCGGAATGACACCGGTAAACCGAAAGGCAGGAACAGGAGAGCGCACG
AGGGAGCCGCCAGGGGGAAACGCCTGGTATCTTTATAGTCCTGTCGGGTT
TCGCCACCACTGATTTGAGCGTCAGATTTCGTGATGCTTGTCAGGGGGGC
GGAGCCTATGGAAAAACGGCTTTGCCGCGGCCCTCTCACTTCCCTGTTAA
GTATCTTCCTGGCATCTTCCAGGAAATCTCCGCCCCGTTCGTAAGCCATT
TCCGCTCGCCGCAGTCGAACGACCGAGCGTAGCGAGTCAGTGAGCGAGGA
AGCGGAATATATCCTGTATCACATATTCTGCTGACGCACCGGTGCAGCCT
TTTTTCTCCTGCCACATGAAGCACTTCACTGACACCCTCATCAGTGCCAA
CATAGTAAGCCAGTATACACTCCGCTAGCGCTGAGGTCTGCCTCGTGAAG
AAGGTGTTGCTGACTCATACCAGGCCTGAATCGCCCCATCATCCAGCCAG
AAAGTGAGGGAGCCACGGTTGATGAGAGCTTTGTTGTAGGTGGACCAGTT
GGTGATTTTGAACTTTTGCTTTGCCACGGAACGGTCTGCGTTGTCGGGAA
GATGCGTGATCTGATCCTTCAACTCAGCAAAAGTTCGATTTATTCAACAA
AGCCACGTTGTGTCTCAAAATCTCTGATGTTACATTGCACAAGATAAAAA
TATATCATCATGAACAATAAAACTGTCTGCTTACATAAACAGTAATACAA
GGGGTGTTATGAGCCATATTCAACGGGAAACGTCTTGCTCGAGGCCGCGA
TTAAATTCCAACATGGATGCTGATTTATATGGGTATAAATGGGCTCGCGA
TAATGTCGGGCAATCAGGTGCGACAATCTATCGATTGTATGGGAAGCCCG
ATGCGCCAGAGTTGTTTCTGAAACATGGCAAAGGTAGCGTTGCCAATGAT
GTTACAGATGAGATGGTCAGACTAAACTGGCTGACGGAATTTATGCCTCT
TCCGACCATCAAGCATTTTATCCGTACTCCTGATGATGCATGGTTACTCA
CCACTGCGATCCCCGGGAAAACAGCATTCCAGGTATTAGAAGAATATCCT
GATTCAGGTGAAAATATTGTTGATGCGCTGGCAGTGTTCCTGCGCCGGTT
GCATTCGATTCCTGTTTGTAATTGTCCTTTTAACAGCGATCGCGTATTTC
GTCTCGCTCAGGCGCAATCACGAATGAATAACGGTTTGGTTGATGCGAGT
GATTTTGATGACGAGCGTAATGGCTGGCCTGTTGAACAAGTCTGGAAAGA
AATGCATAAGCTTTTGCCATTCTCACCGGATTCAGTCGTCACTCATGGTG
ATTTCTCACTTGATAACCTTATTTTTGACGAGGGGAAATTAATAGGTTGT
ATTGATGTTGGACGAGTCGGAATCGCAGACCGATACCAGGATCTTGCCAT
CCTATGGAACTGCCTCGGTGAGTTTTCTCCTTCATTACAGAAACGGCTTT
TTCAAAAATATGGTATTGATAATCCTGATATGAATAAATTGCAGTTTCAT
TTGATGCTCGATGAGTTTTTCTAATCAGAATTGGTTAATTGGTTGTAACA
CTGGCAGAGCATTACGCTGACTTGACGGGACGGCGGCTTTGTTGAATAAA
TCGAACTTTTGCTGAGTTGAAGGATCAGATCACGCATCTTCCCGACAACG
CAGACCGTTCCGTGGCAAAGCAAAAGTTCAAAATCACCAACTGGTCCACC
TACAACAAAGCTCTCATCAACCGTGGCTCCCTCACTTTCTGGCTGGATGA
TGGGGCGATTCAGGCCTGGTATGAGTCAGCAACACCTTCTTCACGAGGCA
GACCTCAGCGCTCAAAGATGCAGGGGTAAAAGCTAACCGCATCTTTACCG
ACAAGGCATCCGGCAGTTCAACAGATCGGGAAGGGCTGGATTTGCTGAGG
ATGAAGGTGGAGGAAGGTGATGTCATTCTGGTGAAGAAGCTCGACCGTCT
TGGCCGCGACACCGCCGACATGATCCAACTGATAAAAGAGTTTGATGCTC
AGGGTGTAGCGGTTCGGTTTATTGACGACGGGATCAGTACCGACGGTGAT
ATGGGGCAAATGGTGGTCACCATCCTGTCGGCTGTGGCACAGGCTGAACG
CCGGAGGATCCTAGAGCGCACGAATGAGGGCCGACAGGAAGCAAAGCTGA
AAGGAATCAAATTTGGCCGCAGGCGTACCGTGGACAGGAACGTCGTGCTG
ACGCTTCATCAGAAGGGCACTGGTGCAACGGAAATTGCTCATCAGCTCAG
TATTGCCCGCTCCACGGTTTATAAAATTCTTGAAGACGAAAGGGCCTCGT
GATACGCCTATTTTTATAGGTTAATGTCATGATAATAATGGTTTCTTAGA
CGTCTTAATTAATCAGGAGAGCGTTCACCGACAAACAACAGATAAAACGA
AAGGCCCAGTCTTTCGACTGAGCCTTTCGTTTTATTTGATGCCTGGCAGT
TCCCTACTCTCGCATGGGGAGACCCCACACTACCATCGGCGCTACGGCGT
TTCACTTCTGAGTTCGGCATGGGGTCAGGTGGGACCACCGCGCTACTGCC
GCCAGGCAAATTCTGTTTTATCAGACCGCTTCTGCGTTCTGATTTAATCT
GTATCAGGCTGAAAATCTTCTCTCATCCGCCAAAACAGCCAAGCTGGAGA
CCGTTTAAACTCAATGATGATGATGATGATGGTCGACGGCGCTATTCAGA
TCCTCTTCTGAGATGAGTTTTTGTTCGGGCCCAAGCTTCGAATTCCCATA
TGGTACCAGCTGCAGATCTCGAGCTCGGATCCATGGTTTATTCCTCCTTA
TTTAATCGATACATTAATATATACCTCTTTAATTTTTAATAATAAAGTTA
ATCGATAATTCCGGTCGAGTGCCCACACAGATTGTCTGATAAATTGTTAA
AGAGCAGTGCCGCTTCGCTTTTTCTCAGCGGCGCTGTTTCCTGTGTGAAA
TTGTTATCCGCTCACAATTCCACACATTATACGAGCCGGATGATTAATTG
TCAACAGCTCATTTCAGAATATTTGCCAGAACCGTTATGATGTCGGCGCA
AAAAACATTATCCAGAACGGGAGTGCGCCTTGAGCGACACGAATTATGCA
GTGATTTACGACCTGCACAGCCATACCACAGCTTCCGATGGCTGCCTGAC
GCCAGAAGCATTGGTGCACCGTGCAGTCGATGATAAGCTGTCAAACCAGA
TCAATTCGCGCTAACTCACATTAATTGCGTTGCGCTCACTGCCCGCTTTC
CAGTCGGGAAACCTGTCGTGCCAGCTGCATTAATGAATCGGCCAACGCGC
GGGGAGAGGCGGTTTGCGTATTGGGCGCCAGGGTGGTTTTTCTTTTCACC
AGTGAGACGGGCAACAGCTGATTGCCCTTCACCGCCTGGCCCTGAGAGAG
TTGCAGCAAGCGGTCCACGCTGGTTTGCCCCAGCAGGCGAAAATCCTGTT
TGATGGTGGTTGACGGCGGGATATAACATGAGCTGTCTTCGGTATCGTCG
TATCCCACTACCGAGATATCCGCACCAACGCGCAGCCCGGACTCGGTAAT
GGCGCGCATTGCGCCCAGCGCCATCTGATCGTTGGCAACCAGCATCGCAG
TGGGAACGATGCCCTCATTCAGCATTTGCATGGTTTGTTGAAAACCGGAC
ATGGCACTCCAGTCGCCTTCCCGTTCCGCTATCGGCTGAATTTGATTGCG
AGTGAGATATTTATGCCAGCCAGCCAGACGCAGACGCGCCGAGACAGAAC
TTAATGGGCCCGCTAACAGCGCGATTTGCTGGTGACCCAATGCGACCAGA
TGCTCCACGCCCAGTCGCGTACCGTCTTCATGGGAGAAAATAATACTGTT
GATGGGTGTCTGGTCAGAGACATCAAGAAATAACGCCGGAACATTAGTGC
AGGCAGCTTCCACAGCAATGGCATCCTGGTCATCCAGCGGATAGTTAATG
ATCAGCCCACTGACGCGTTGCGCGAGAAGATTGTGCACCGCCGCTTTACA
GGCTTCGACGCCGCTTCGTTCTACCATCGACACCACCACGCTGGCACCCA
GTTGATCGGCGCGAGATTTAATCGCCGCGACAATTTGCGACGGCGCGTGC
AGGGCCAGACTGGAGGTGGCAACGCCAATCAGCAACGACTGTTTGCCCGC
CAGTTGTTGTGCCACGCGGTTGGGAATGTAATTCAGCTCCGCCATCGCCG
CTTCCACTTTTTCCCGCGTTTTCGCAGAAACGTGGCTGGCCTGGTTCACC
ACGCGGGAAACGGTCTGATAAGAGACACCGGCATACTCTGCGACATCGTA
TAACGTTACTGGTTTCACATTCACCACCCTGAATTGACTCTCTTCCGGGC
GCTATCATGCCATACCGCGAAAGGTTTTGCACCATTCGATGGTGTCAACG
TAAATGCATGCCGCTTCGCCTTCGCGCGCGAATTGATCTGCTGCCTCGCG
CGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGG
GCGCGTCAGCGGGTGTTGGCGGGGCCGGCCTCG

The 'tesA, fadD, and atfA1 genes were amplified using high fidelity Phusion™ polymerase (New England Biolabs, Inc., Ipswich, Mass.), with the following primers from their respective plasmids, pETDuet-1-'tesA, pHZ1.61, and pHZ1.97-atfA1:

‘tesAForward-

(SEQ ID NO: 12)

5′-CTCTAGAAATAATTTAACTTTAAGTAGGAGAUAGGTACCCATGGCGG
ACACGTTATTGAT
‘tesAReverse-

(SEQ ID NO: 13)

5′-CTTCGAATTCCATTTAAATTATTTCTAGAGTCATTATGAGTCATGAT
TTACTAAAGGC
fadDForward-

(SEQ ID NO: 14)

5′-CTCTAGAAATAATTTTAGTTAAGTATAAGAAGGAGATATACCATGGT
GAAGAAGGTTTGGCTTAA
fadDReverse-

(SEQ ID NO: 15)

5′-CTTCGAATTCCATTTAAATTATTTCTAGAGTTATCAGGCTTTATTGT
CCAC
atfA1Forward-

(SEQ ID NO: 16)

5′-CTCTAGAAATAATTTAGTTAAGTATAAGAAGGAGATATACAT
atfA1Reverse-

(SEQ ID NO: 17)

5′-CTTCGAATTCCATTTAAATTATTTCTAGAGTTACTATTTAATTCCTG
CACCGATTTCC

Insertion of 'tesA into pACYC-Ptrc Plasmid

Using NcoI and EcoRI sites on both the insert and vector, the 'tesA PCR product amplified from pETDuet-1-'tesA was cloned into the initial position of pACYC-PTrc vector (SEQ ID NO:11). A T4 DNA ligase (New England Biolabs, Ipswich, Mass.) was then used to ligate the pACYC-PTrc vector and 'tesA, producing a pACYC-PTrc-'tesA plasmid. Following overnight ligation, the DNA product was transformed into Top 10 One Shot® cells (Invitrogen, Carlsbad, Calif.). The 'tesA insertion into the pACYC-PTrc vector was confirmed by restriction digestion. An SwaI restriction site as well as overlapping fragments for In-Fusion™ cloning (Clontech, Mountain View, Calif.) was also created at the 3′ end of the 'tesA insert.

Construction of pACYC-PTrc-'tesA-fadD-atfA1

The pACYC-PTrc-'tesA plasmid was then subject to an overnight digestion by SwaI. fadD amplified from pHZ1.61 was cloned after the 'tesA gene using In-Fusion™ cloning. This insertion of fadD was verified with restriction digestion. The insertion of fadD destroys the SwaI site following the 'tesA gene, but recreates a new SwaI site at the 3′ end of fadD.

The pACYC-PTrc-'tesA fadD plasmid was again linearized by SwaI, and atfA1 amplified from pHZ1.97-atfA1 was cloned after the fadD gene using In-Fusion™ cloning. The proper insertion of atfA1 was verified by restriction digestion.

Construction of the pOP-80 (pCL) plasmid

A low copy plasmid pCL1920 (in accordance with Lerner et al., Nucleic Acids Res. 18:4631 (1990)) carrying a strong transcriptional promoter was digested with restriction enzymes AflII and SfoI (New England BioLabs Inc. Ipswich, Mass.). Three DNA sequence fragments were produced by this digestion, among which a 3737 by fragment was gel-purified using a gel-purification kit (Qiagen, Inc. Valencia, Calif.).

In parallel, a fragment containing the Trc-promoter and lad region from the commercial plasmid pTrcHis2 (Invitrogen, Carlsbad, Calif.) was amplified by PCR using the following primers:

LF302:

(SEQ ID NO: 18)

5′-ATATGACGTCGGCATCCGCTTACAGACA-3′
LF303

(SEQ ID NO: 19)

(5′-AATTCTTAAGTCAGGAGAGCGTTCACCGACAA-3′.

These two primers also introduced recognition sites for ZraI (gacgtc) and AflII (cttaag), at the end of the PCR product. The PCR product was purified using a PCR-purification kit (Qiagen, Inc. Valencia, Calif.) and digested with ZraI and AflII following the recommendations of the supplier (New England BioLabs Inc., Ipswich, Mass.). The digested PCR product was then gel-purified and ligated with the 3737 by DNA sequence fragment derived from pCL1920. The ligation mixture was transformed in TOP10® chemically competent cells (Invitrogen, Carlsbad, Calif.), and the transformants were plated on Luria agar plates containing 100 μg/mL spectinomycin. After overnight incubation at 37° C., a number of colonies were visible. A select number of these colonies were purified, analyzed with restriction enzymes, and sequenced. One of the plasmids was retained and given the name pOP-80.

Construction of pCL-TFW-atfA1

The operon 'tesA-fadD-atfA1 was removed from pACYC-'tesA-fadD-atfA1 using restriction digestion with MluI and EcoRI (New England Biolabs, Inc., Ipswich, Mass.). It was then cloned into complementary sites on pOP-80 to create the plasmid pCL-TFW-atfA1.

Integration of the PTrc-'tesA-fadD-atfA1 operon into the E. coli MG1655 ΔfadE ΔfhuA Chromosome at the lacI-lacZ Locus

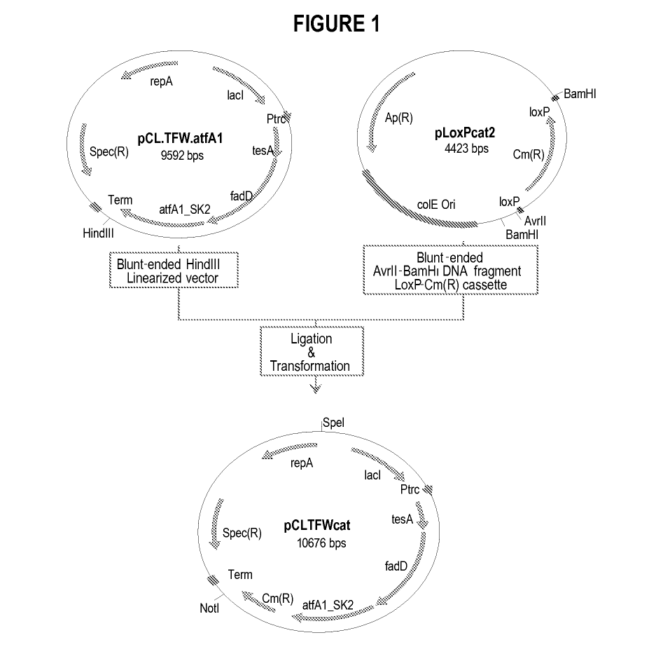

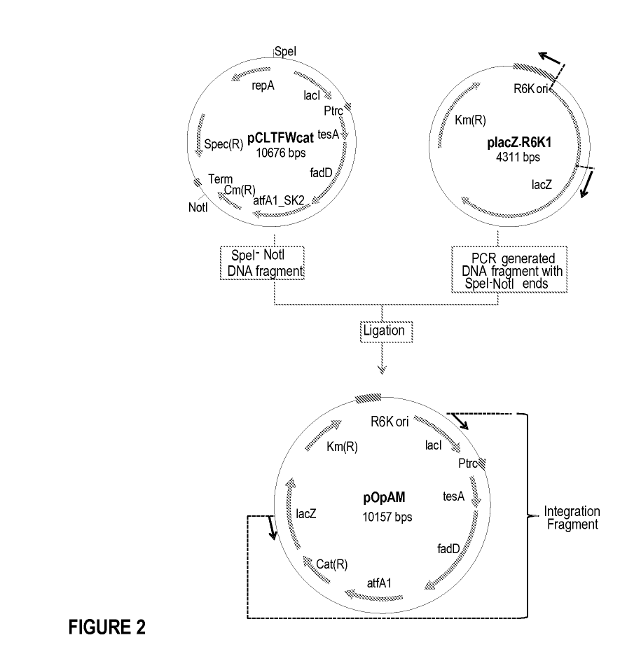

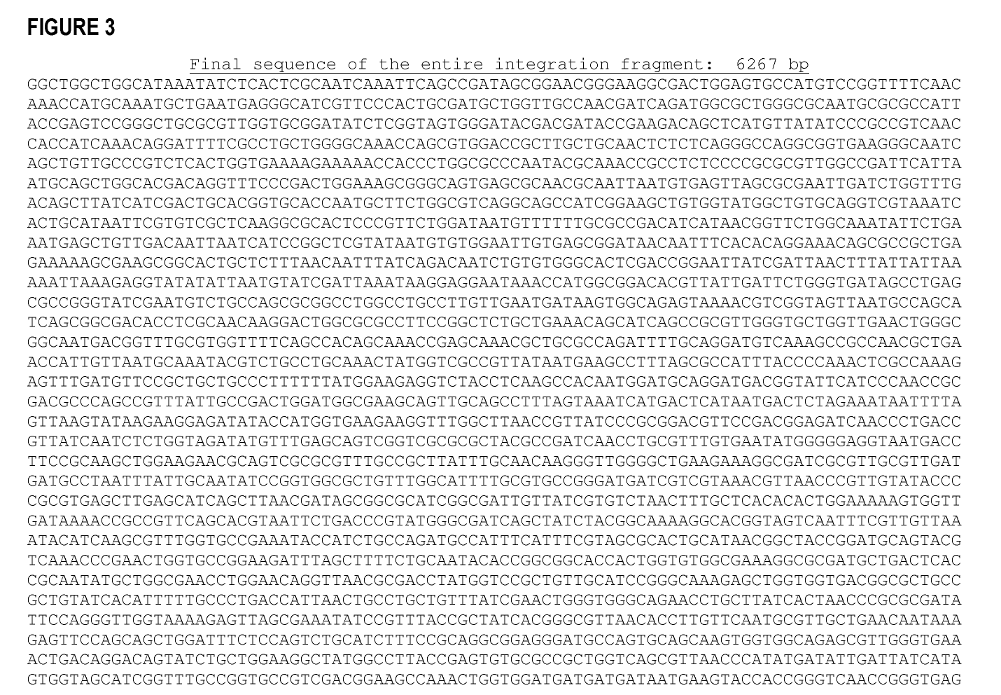

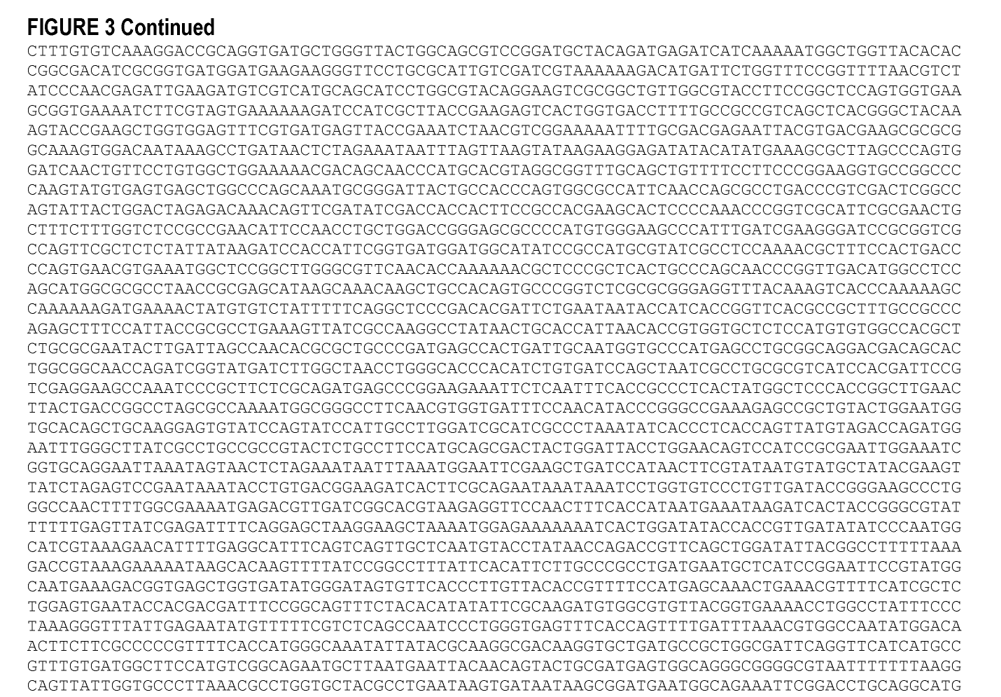

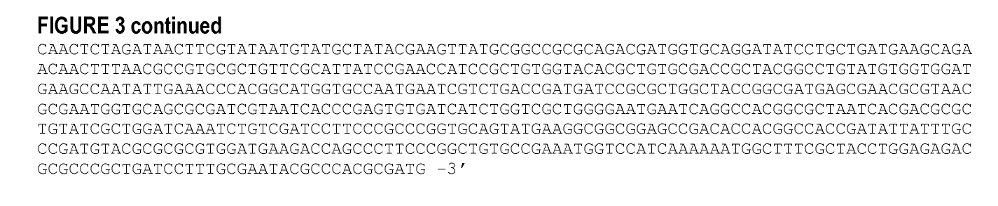

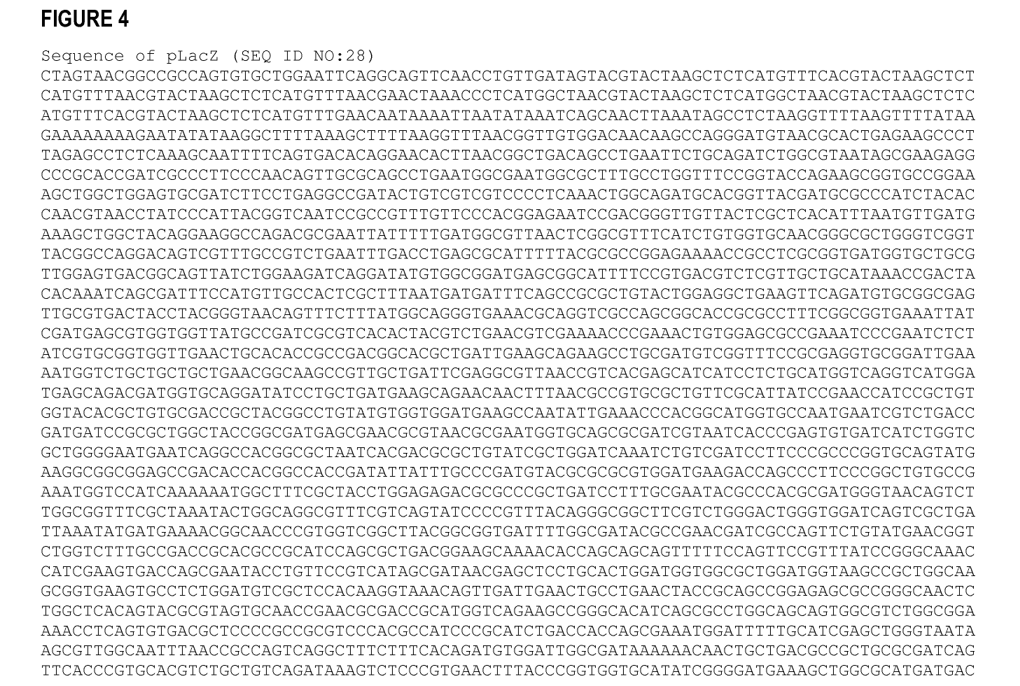

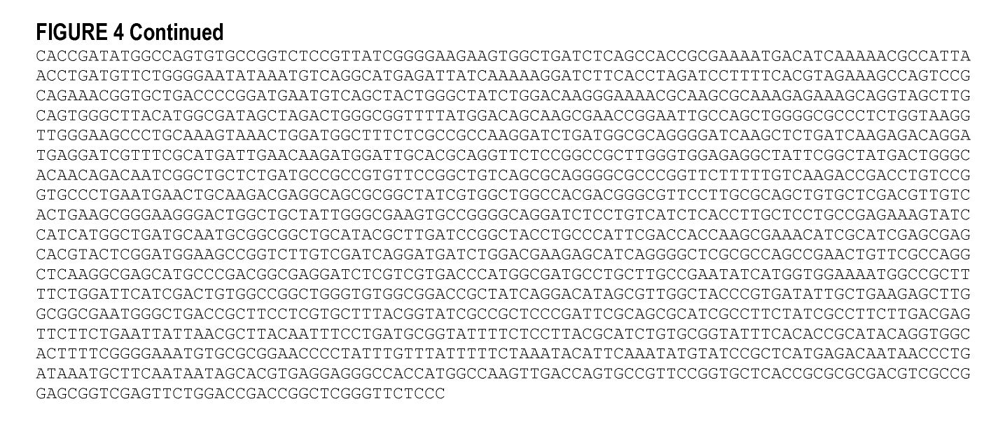

Plasmid pCL-TFW-atfA1 was digested with restriction enzyme HindIII (New England Biolabs, Inc., Ipswich). In parallel, a chloramphenicol gene cassette was obtained from plasmid pLoxPcat2 (Genbank Accession No. AJ401047) by digestion with restriction enzymes BamHI and AvrII (New England Biolabs, Inc., Ipswich, Mass.). Both DNA fragments were blunt-ended using the DNA polymerase Klenow fragment. The resulting fragments were ligated and transformed to generate plasmid pCLTFWcat (see, FIG. 1).

Plasmid placZ constructed and synthesized by DNA2.0 (Menlo Park, Calif.) in accordance to the sequence of SEQ ID NO:28 was used as a template for PCR amplification. PCR primers LacZFnotl and pKDRspeI were designed to create restriction sites for the NotI and SpeI, respectively:

(SEQ ID NO: 20)

| | |
|---|---|
| LacZFnotI | 5′-CAACCAGCGGCCGCGCAGACGATGGTGCAGGATATC |

(SEQ ID NO: 21)

| | |
|---|---|
| pKDRspeI | 5′-CCACACACTAGTCAGATCTGCAGAATTCAGGCTGTC |

The resulting DNA fragment was ligated with a DNA fragment from plasmid pCLTFWcat digested with SpeI and NotI enzymes.

The ligation mixture was used as a template for another PCR reaction using primers lacIF and lacZR located on the lad and lacZ regions.

| | |
|---|---|
| lacIF 5′-GGCTGGCTGGCATAAATATCTC | (SEQ ID NO: 22) |
| lacZR 5′-CATCGCGTGGGCGTATTCG | (SEQ ID NO: 23) |

The resulting PCR product (“Integration Cassette”) contains approximately 500 bases of homology to lad or lacZ at each end. This PCR product was used to transform E. coli MG1655 ΔfadE ΔfhuA (DV2) cells that were made hypercompetent with plasmid pKD46 (see, Example 2).

This example demonstrate the construction of E. coli MG1655 ΔfadE, ΔfhuA, lacZ:: 'tesA fadD atfA1, which is a genetically engineered microorganism in which a fatty acid degradation enzyme and an outer membrane protein receptor for ferrichrome are attenuated and nucleotide sequences encoding a thioesterase, an acyl-CoA synthase, and an ester synthase are integrated into the microorganism's chromosome. This strain was given the name “IDV2.”

### Example 4

### Production of E. coli MG1655 ΔfadE ΔfhuA ΔfabR

This example describes the construction of a genetically engineered microorganism in which the expression of a DNA-binding transcriptional repressor was attenuated.

The fabR gene of E. coli MG1655, which encodes a DNA-binding transcriptional repressor (GenBank Accession No. AAC76945; REFSEQ: accession U00096.2), was deleted from strain DV2 (see, Example 2), using the Lambda Red system described in Dastsenko et al., supra, with the following modifications:

Two primers were used to create the deletion, along with a new Km^{R} marker:

Del-fabR1:

(SEQ ID NO: 24)

5′-CGTACCTCTATCTTGATTTGCTTGTTTCATTACTCGTCCTTCACATT
TCCGTGTAGGCTGGAGCTGCTTCG
Del-fabR2:

(SEQ ID NO: 25)

5′-ATGTTTTATTGCGTTACCGTTCATTCACAATACTGGAGCAATCCAGT
ATGATTCCGGGGATCCGTCGACC

Confirmation of the deletion of fabR was performed using the following primers:

| | |
|---|---|
| fabR-F 5′-AACCGGCCAAAGAATTGCAG | (SEQ ID NO: 26) |
| fabR-R 5′-TAAGCCAGCAACTAACGCCA | (SEQ ID NO: 27) |

After the fabR deletion was confirmed, the Km^{R} marker was removed from one colony using the pCP20 plasmid as described in Datsenko et al., supra. The resulting MG1655 E. coli strain having fadE, fhuA, and fabR gene deletions was named E. coli MG1655 ΔfadE ΔfhuA ΔfabR, or E. coli MG1655 DV2 ΔfabR.

### Example 5

### Production of E. coli MG1655 ΔfadE ΔfhuA ΔfabR, lacZ:: tesA fadD atfA1

This example describes the construction of a genetically engineered microorganism in which the nucleotide sequences encoding a thioesterase, an acyl-CoA synthase, and an ester synthase are integrated into the microorganism's chromosome. In addition, the genes of the microorganism encoding a fatty acid degradation enzyme, an outer membrane protein receptor for ferrichrome, and a DNA-binding transcriptional repressor are attenuated.

The operon 'tesA-fadD-atfA1 can be obtained from the pACYC-PTrc-'tesA-fadD-atfA1 plasmid described in Example 3. This operon was integrated into the E. coli MG1655 DV2-fabR strain of Example 4. The resulting strain was named E. coli MG1655 ΔfadE ΔfhuA ΔfabR, lacZ:: tesA fadD atfA1, or E. coli MG1655 IDV2 ΔfabR.

### Example 6

### Construction of a Genetically Engineered Cyanobacterium Host Cell

A vector can be constructed for homologous recombination into the Synechococcus sp. PCC7002 plasmid pAQ1 (genbank accession NC_{—}0050525) using 500 by homologous regions corresponding to positions 3301-3800 and 3801-4300 of pAQ1.

As a selectable marker, a spectinomycin resistance cassette (containing aminoglycoside 3′ adenylyltransferase, aad, promoter, gene and terminator), for example, one obtained from plasmid pCL1920 (in accordance with Lerner et al., Nucleic Acids Res. 18:4631 (1990), described in Example 3), can be added between the homologous regions.

A plasmid, such as pACYC177, can be prepared in accordance with Chang, et al. J. Bacteriol. 134: 1141-1156 (1978). The promoter and ribosome binding site of aminoglycoside phosphotransferase, aph, can be added followed by an appropriate and unique cloning sites that are, for example, NdeI and EcoRI recognition sequences. This complete integration cassette can be synthesized and cloned into a pUC19 vector (New England Biolabs, Inc., Ipswich, Mass.). The resulting plasmid, pLS9-7002, will allows (i) cloning and expression of a foreign gene and (ii) delivery and stable in-vivo integration into Synechococcus sp. PCC7002 plasmid pAQ1.

A synthetic operon consisting of a thioesterase gene (e.g., 'tesA from E. coli), a fatty acyl-CoA synthetase gene (e.g., fadD from E. coli) and an ester synthase gene (e.g., atfA1 from Alcanivorax borkumensis strain SK2) can be created in accordance with the disclosure of Example 3. This operon can then be cloned into the NdeI and EcoRI sites of pLS9-7002 downstream of the aph promoter and ribosome binding site.

The resulting plasmid can then be transformed into Synechococcus sp. PCC7002 using a method described by Stevens et al., PNAS 77:6052-56 (1980). Stable integrants can be identified and selected for on ATCC 1047 medium supplemented with 15 μg/mL spectinomycin. Specifically, 1 L of ATCC 1047 medium contains: 40 mg MgSO₄×7H₂O, 20 mg CaCl₂×2H₂O, 750 mg NaNO₃, 2 mg K₂HPO₄, 3.0 mg citric acid, 3.0 mg ferric ammonium citrate, 0.5 mg EDTA, 20 mg Na₂CO₃, 2.86 mg H₃BO₃, 1.81 mg MnCl₂, 0.22 mg ZnSO₄, 0.04 mg Na₂MoO₄, 0.08 mg CuSO₄, 0.05 mg Co(NO₃)₂, 0.02 mg vitamin B12, 10 g agar and 750 mL sea water. Spectinomycin resistant colonies can be restreaked several times on the ATCC medium 1047 supplemented with spectinomycin and tested for isogenic integration of the 'tesA-fadD-atfA1 operon using PCR with the following primers:

| | |
|---|---|
| pAQ1-U: ATGTCTGACAAGGGGTTTGACCCCT | (SEQ ID NO: 29) |
| pAQ1-D: GCACATCCTTATCCAATTGCTCTAG | (SEQ ID NO: 30) |

Complete isogenic 'tesA-fadD-atfA1 integrants can then be grown in a 50 mL liquid ATCC 1047 medium containing spectinomycin in a 500 mL shake flasks with appropriate aeration and illumination at 30° C. for up to 7 d. Methanol (2% v/v) can be added 5-15 h after inoculation. Culture aliquots can be extracted at various intervals with ethyl acetate, and the extracts can be analyzed for fatty ester production

### Example 7

### Photoautotrophic Production of Fatty Esters in the Cyanobacterium Synechococcus elongatus PCC7942

A vector can be constructed for homologous recombination into the Synechococcus elongatus PCC7942 genome (genbank accession CP_{—}000100) using 800 by homologous regions corresponding to positions 2577844-2578659 and 2578660-2579467 of CP_{—}000100. This chromosomal location is known as neutral site one (NS1) (Mackey et al., Meth. Mol. Biol. 362:115-129 (2007).

As a selectable marker, a spectinomycin resistance cassette (containing aminoglycoside 3′-adenylyltransferase, aad, promoter, gene and terminator), for example, one obtained from plasmid pCL1920 (in accordance with Lerner et al., Nucleic Acids Res. 18:4631 (1990), described in Example 3), can be added between the homologous regions. Additionally, appropriate and unique cloning sites, for example NdeI and EcoRI recognition sites, can be added.

This integration cassette can be synthesized and cloned into pUC19 (New England Biolabs, Inc., Ipswich, Mass.). The resulting plasmid, pLS9-7942-NS1, allows (i) cloning and expression of a foreign gene and (ii) delivery and stable integration into the Synechococcus elongatus PCC7942 genome.

A synthetic operon consisting of a thioesterase gene (e.g., 'tesA from E. coli), a fatty acyl-CoA synthetase gene (e.g., fadD from E. coli) and an ester synthase gene (e.g., atfA1 from Alcanivorax borkumensis strain SK2) can be created, including a PTrc promoter and ribosomal binding site, in accordance with the disclosure of Example 3. This operon can then be cloned into the NdeI or EcoRI site of pLS9-7942-NS1. The resulting plasmid can be transformed into S. elongatus PCC7942 in accordance with a method described by Mackey et al., supra.

Stable integrants can be identified and selected for on a BG-11 medium supplemented with 4 μg/mL spectinomycin. Specifically, 1 L of BG-11 medium contains: 75 mg MgSO₄×7H₂O, 36 mg CaCl₂×2H₂O, 1.5 g NaNO₃, 40 mg K₂HPO₄, 6.0 mg citric acid, 6.0 mg ferric ammonium citrate, 1.0 mg EDTA, 20 mg Na₂CO₃, 2.86 mg H₃BO₃, 1.81 mg MnCl₂, 0.22 mg ZnSO₄, 0.04 mg Na₂MoO₄, 0.08 mg CuSO₄, 0.05 mg Co(NO₃)₂ and 10 g agar. Spectinomycin resistant colonies can be restreaked several times on the BG-11 medium supplemented with spectinomycin and tested for isogenic integration of the PTrc-'tesA-fadD-atfA1 operon by PCR, using the following primers:

| | |
|---|---|
| NS1-U: GATCAAACAGGTGCAGCAGCAACTT | (SEQ ID NO: 31) |
| NS1-D: ATTCTTGACAAGCGATCGCGGTCAC | (SEQ ID NO: 32) |

Complete isogenic PTrc-'tesA-fadD-AtfA1 integrants can then grown in a 50 mL liquid BG-11 medium supplemented with spectinomycin in a 500 mL shake flask with appropriate aeration and illumination at 30° C. for 5-7d. Methanol (2% v/v) can be added 5-15 h after inoculation. Culture aliquots can be extracted at various intervals with ethyl acetate, and the extracts can be analyzed for fatty ester production.

### Example 8

### Photoautotrophic Production of Fatty Esters in the Cyanobacterium Synechocystis sp. PCC6803

A vector can be constructed for homologous recombination into the Synechocystis sp. PCC6803 genome (genbank accession BA_{—}000022) using 1300 to 1700 by homologous regions corresponding to positions 2299015-2300690 and 2300691-2302056 of BA_{—}000022, respectively. This chromosomal location is known as neutral site RS1/2 (Shao et al., Appl. Environ. Microbiol. 68:5026-33 (2002)).

A plasmid, such as pACYC177, can be prepared in accordance with Chang, et al. J. Bacteriol. 134: 1141-1156 (1978). As a selectable marker, a kanamycin resistance cassette (containing aminoglycoside phosphotransferase, aph, promoter, gene and terminator) can be derived from the pACY177 plasmid, and it can be added between the homologous regions. Additionally, appropriate and unique cloning sites, for example, NdeI and XbaI recognitions sites can be added. This integration cassette can be synthesized and cloned into pUC19 (New England Biolabs, Inc., Ipswich, Mass.). The resulting plasmid, pLS9-6803-RS, allows (i) cloning and expression of a foreign gene and (ii) delivery and stable integration into the Synechocystis sp. PCC6803 genome.

A synthetic operon consisting of a thioesterase gene (e.g., 'tesA from E. coli), a fatty acyl-CoA synthetase gene (e.g., fadD from E. coli) and an ester synthase gene (e.g., atfA1 from Alcanivorax borkumensis strain SK2) can be created, including a PTrc promoter and ribosomal binding site, in accordance with the disclosure of Example 3. This operon can then be cloned into the NdeI or XbaI site of pLS9-6803-RS. The resulting plasmid can be transformed into Synechocystis sp. PCC6803 in accordance with a method described by Zang et al. J. Microbiol., 45:241-45 (2007).

Stable integrants are selected for on a BG-11 medium supplemented with 10 μg/mL kanamycin. Specifically, 1 L of the BG-11 medium contains: 75 mg MgSO₄×7H₂O, 36 mg CaCl₂×2H₂O, 1.5 g NaNO₃, 40 mg K₂HPO₄, 6.0 mg citric acid, 6.0 mg ferric ammonium citrate, 1.0 mg EDTA, 20 mg Na₂CO₃, 2.86 mg H₃BO₃, 1.81 mg MnCl₂, 0.22 mg ZnSO₄, 0.04 mg Na₂MoO₄, 0.08 mg CuSO₄, 0.05 mg Co(NO₃)₂ and 10 g agar. Kanamycin resistant colonies are restreaked several times on the BG-11 medium supplemented with kanamycin and tested for isogenic integration of the PTrc-'tesA-fadD-atfA1 operon using PCR with the following primers:

| | |
|---|---|
| RS1: ATTCAATACACCCCCCTAGCCGATC | (SEQ ID NO: 33) |
| RS2: TAAGGGTGGTGGGAAAAATGGGCCA | (SEQ ID NO: 34) |

Complete isogenic PTrc-'tesA-fadD-AtfA1 integrants are then grown in a 50 mL liquid BG-11 medium supplemented with kanamycin in a 500 mL shake flask with appropriate aeration and illumination at 30° C. for 5-7d. Methanol (2% v/v) can be added 5-15 h after inoculation. Culture aliquots can be extracted at various time points with ethyl acetate, and the extracts can be analyzed for fatty ester production.

### Example 9

### Production of Biodiesel by Fermentation

This example demonstrates processes that can be used to produce a fatty ester composition using the genetically modified microorganisms described herein. A fermentation and recovery process was used to produce biodiesel of commercial grade quality by fermentation of carbohydrates. The fermentation process produced a mix of fatty acid methyl esters (FAME) and fatty acid ethyl esters (FAEE) for use as a biodiesel using the genetically engineered microorganisms described in Examples 1-5.

### Fermentation

The following fermentation protocol was developed for the production of FAME and fatty acid ethyl esters (FAEE) in lab scale fermentors of 2 L to 5 L of volume. These exemplary protocols can be scaled up as any other E. coli fermentation, using methods well known to one of ordinary skill in the art.

Fermentations were carried out in 2 L fermentors. E. coli cells from a frozen stock were grown in a defined media consisting of: 4.54 g/L of K₂HPO₄ trihydrate, 4 g/L of (NH₄)₂SO₄, 0.15 g/L of MgSO₄ heptahydrate, 20 g/L of glucose, 200 mM of Bis-Tris buffer (pH 7.2), 1.25 mL/L of trace mineral solution and 1.25 mL/L of a vitamin solution. The trace metals solution was composed of 27 g/L of FeCl₃.6H₂O, 2 g/L of ZnCl₂.4H₂O, 2 g/L of CaCl₂.6H₂O, 2 g/L of Na₂MoO₄.2H₂O, 1.9 g/L of CuSO₄.5H₂O, 0.5 g/L of H₃BO₃, and 100 mL/L of concentrated HCl. The vitamin solution was composed of 0.42 g/L of riboflavin, 5.4 g/L of pantothenic acid, 6 g/L of niacin, 1.4 g/L of pyridoxine, 0.06 g/L of biotin, and 0.04 g/L of folic acid.

50 mL of cultures of IDV2 and IDV2 ΔfabR strains (described in Examples 3 and 5, respectively) were grown overnight and subsequently used to inoculate 1 L of medium containing: 0.5 g/L (NH₄)₂SO₄, 4.9 g/L KH₂PO₄, 0.15 g/L MgSO₄ heptahydrate, 2 g/L glucose, 1.25 mL/L of trace mineral solution and 1.25 mL/L of vitamin solution, in a fermentor with temperature, pH, agitation, aeration and dissolved oxygen control. The preferred conditions for the fermentation were 32° C., pH 6.8 and dissolved oxygen (DO) equal to 30% of saturation. The pH was maintained by addition of NH₄OH, which also served as nitrogen source for cell growth. The glucose level in the culture was monitored using methods known to those skilled in the art. When the initial glucose was almost consumed, a feed consisting of 600 g/L glucose, 3.9 g/L MgSO₄ heptahydrate and 10 mL/L of the trace minerals solution was supplied to the fermentor. The feed rate was set up to allow for a cell growth rate of 0.3 h⁻¹, up to a maximum of 10 g glucose/L/h, at which point it was fixed. This rate was maintained for the remaining duration of the fermentation as long as glucose did not accumulate in the fermentor. By avoiding glucose accumulation, it was possible to reduce or eliminate the formation of by-products such as acetate, formate and ethanol, which are commonly produced by E. coli. In the early phases of the growth, the production of FAME was induced by the addition of 1 mM IPTG and 20 mL/L of pure methanol. The fermentation was continued for a period of 3 days. Methanol was added several times during the run to replenish what was consumed by the cells for the production of FAME, but mostly to replace methanol lost by evaporation in the off-gas. The additions were targeted to maintain the concentration of methanol in the fermentation broth between 10 and 30 mL/L, to allow efficient production while avoiding inhibition of cell growth.

The progression of the fermentation was followed by measurements of OD₆₀₀ (optical density at 600 nm), glucose consumption, and ester production. This fermentation protocol was scaled up to a 700 L fermentor using methods well known in the art.

### Analysis

Glucose consumption throughout the fermentation was analyzed by High Pressure Liquid Chromatography (HPLC). The HPLC analysis was performed according to methods commonly used for some sugars and organic acids in the art, using the following conditions: Agilent HPLC 1200 Series with Refractive Index detector; Column: Aminex HPX-87H, 300 mm×7.8 mm; column temperature: 350 C; mobile phase: 0.01M H₂SO₄ (aqueous); flow rate: 0.6 mL/min; injection volume: 20 μL.

The production of fatty acid methyl and ethyl esters was analyzed by gas chromatography with flame ionization detector (GC-FID). Samples from fermentation broth were extracted with ethyl acetate in a ratio of 1:1 vol/vol. After strong vortexing, the samples were centrifuged, and the organic phase was analyzed by gas chromatography (GC). The analysis conditions were as follows: instrument: Trace GC Ultra, Thermo Electron Corporation with Flame ionization detector (FID) detector; column: DB-1 (1% diphenyl siloxane; 99% dimethyl siloxane) CO1 UFM 1/0.1/5 01 DET from Thermo Electron Corporation, phase pH 5, FT: 0.4 μm, length 5 m, id: 0.1 mm; inlet conditions: 250° C. splitless, 3.8 min 1/25 split method used depending upon sample concentration with split flow of 75 mL/min; carrier gas, flow rate: Helium, 3.0 mL/min; block temperature: 330° C.; oven temperature: 0.5 minute hold at 50° C., 100° C./minute to 330° C. 0.5 minute hold at 330° C.; detector temperature: 300° C.; injection volume: 2 μL; run time/flow rate: 6.3 min/3.0 mL/min (splitless method), 3.8 min/1.5 mL/min (split 1/25 method), 3.04 min/1.2 mL/min (split 1/50 method).

### Recovery

After fermentation, the fatty ester composition can be separated from the fermentation broth by several different methods well known in the art. After the completion of the fermentation, the broth was centrifuged to separate a first light phase containing the methylesters from a first heavy phase consisting of water, salts and the microbial biomass. The first light phase was centrifuged a second time to recover the biodiesel and to separate a second light phase (which consisted of a mixture of ethers) from a second heavy phase.

Centrifugation was performed in disk-stacked continuous centrifuges of pilot scale capacity (fixed centrifugal force about 10,000 g) with flows from about 1 to about 5 L per min. The same centrifuge was used each time for the first and second steps. Normal adjustments known in the art to centrifugation configuration and conditions (gravity ring size, back pressure in outlets, flow) were undertaken in each case to achieve the most favorable separation in terms of recovery efficiency and cleanness of the product. For the first centrifugation step, the fermentation broth was sent directly from the fermentor to the centrifuge without any physical or chemical adjustments.

In instances where it was more difficult to break the emulsion and obtain clear oil, additional pretreatments were applied to the light phase to help with the separation during the second centrifugation step. These treatments consisted of the following: heating to 60 to 80° C., adjusting the pH to 2.0 to 2.5 with sulfuric acid, and addition of demulsifiers (ARB-8285 (Baker Hughes, Houston, Tex.), less than 1% of the emulsion/light phase volume). The temperature was held for 1 to 2 h before the second centrifugation.

### Polishing

The composition obtained from the harvesting step had characteristics that are very close to the regulatory standards for biodiesel. The inherent properties of this composition, as well as the properties related to purity, met the regulatory standards for biodiesel, including cetane number, kinematic viscosity, flash point, oxidation stability, copper corrosion, free and total glycerin, methanol, phosphorous, sulfate, K⁺ and Na⁺ content. However, the composition obtained from the harvesting step can, in certain embodiments, be subjected to optional minor further purification steps to eliminate other impurities.

The following optional operations were performed on the clarified composition: elimination of free fatty acids by lime washing, removal of excess calcium by dilute acid wash, removal of potential excess free acid by water washing, and final drying using Magnesol™ D60 (The Dallas Group, Inc., Whitehouse, N.J.).

### Results

Fermentation runs: the IDV2 and IDV2 ΔfabR strains were grown according to the protocol herein in 2 L, 5 L and 700 L fermentors. Representative results obtained are presented in Table 7. The yield is expressed as the grams of product obtained per 100 g of carbon source used.

TABLE 7

| Parameter | IDV2 | IDV2 ΔfabR |
|---|---|---|
| FAME concentration (g/L) | 34.1 | 35.3 |
| FFA concentration (g/L) | 3.5 | NA |
| Yield of FAME on glucose (%) | 11.8 | 12.9 |
| Yield of FAME + FFA on glucose (%) | 13.0 | NA |
| Percentage unsaturated | 32.8 | 51.5% |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing a fatty acid derivative the method comprising culturing a host cell in the presence of a carbon source, wherein the host cell is genetically engineered to overexpress a gene encoding a thioesterase, a gene encoding an acyl-CoA synthase, and a gene encoding an ester synthase.

2. The method of claim 1, further comprising isolating the fatty acid derivative.

3. The method of claim 1, further comprising culturing the host cell in the presence of an alcohol.

4. The method of claim 1, wherein the gene encoding a thioesterase is tesA, 'tesA, fatB, fatB2, fatB3, fatA1, or fatA.

5. The method of claim 1, wherein the gene encoding an acyl-CoA synthase is fadD, fadK, BH3103, pfl-4354, EAV15023, fadD1, fadD2, RPC_{—}4074, fadDD, faa39, the gene that encodes the protein of GenBank Accession No. ZP_{—}01644875, or yhfL.

6. The method of claim 1, wherein the gene encoding an ester synthase is obtained from Acinetobacter sp., Alcanivorax borkumensis, Alcaligenes eutrophus, Mortierella alpina, Cryptococcus curvatus, Arabidopsis thaliana, Fundibacter jadensis, Pseudomonas aeruginosa, Rhodococcus opacus, Marinobacter hydrocarbonoclastics, Saccharomyces cerevisiae, Homo sapiens, or Simmondis chinensis.

7. The method of claim 6, wherein the gene encoding an ester synthase is wax/dgat, a gene encoding a wax synthase, or a gene encoding a bifunctional ester synthase/acyl-CoA:diacylglycerol acyltransferase.

8. The method of claim 1, wherein the host cell is genetically engineered to express, relative to a wild type host cell, a decreased level of at least one of a gene encoding an acyl-CoA dehydrogenase, a gene encoding an outer membrane protein receptor, and a gene encoding a transcriptional regulator of fatty acid biosynthesis.

9. The method of claim 1, wherein the host cell is genetically engineered such that at least one of a gene encoding an acyl-CoA dehydrogenase, a gene encoding an outer membrane protein receptor, and a gene encoding a transcriptional regulator of fatty acid biosynthesis is deleted.

10. The method of claim 8 or 9, wherein the gene encoding an acyl-CoA dehydrogenase is fadE.

11. The method of claim 8 or 9, wherein the gene encoding the outer membrane protein receptor is a gene encoding an outer membrane ferrichrome transporter.

12. The method of claim 11, wherein the gene encoding the outer membrane ferrichrome transporter is fhuA.

13. The method of claim 8 or 9, wherein the gene encoding the transcriptional regulator of fatty acid biosynthesis encodes a DNA transcriptional repressor.

14. The method of claim 13, wherein the gene encoding the DNA transcriptional repressor is fadR.

15. The method of any one of claims 1-14, wherein the fatty acid derivative is a fatty ester.

16. The method of any one of claims 1-15, wherein the host cell is cultured in a culture medium comprising an initial concentration of the carbon source of about 2 g/L or higher.

17. (canceled)

18. The method of any one of claims 1-15, wherein the host cell is cultured in a culture medium comprising an initial concentration of the carbon source of about 100 g/L or lower.

19. (canceled)

20. (canceled)

21. (canceled)

22. The method of any one of claims 1-15, further comprising monitoring the level of the carbon source in the culture medium.

23. The method of claim 22, wherein a supplemental carbon source is added to maintain a carbon source concentration of about 2 g/L or more.

24. (canceled)

25. The method of claim 22, wherein a supplemental carbon source is added to maintain a carbon source concentration of about 5 g/L or less.

26. (canceled)

27. (canceled)

28. The method of claim 1, wherein the carbon source is glucose.

29. The method of claim 3, wherein the alcohol is methanol or ethanol.

30. The method of claim 29, wherein the methanol or ethanol is present at a concentration of about 1 mL/L or more.

31. (canceled)

32. The method of claim 29, wherein the methanol or ethanol is present at a concentration of about 100 mL/L or less.

33. (canceled)

34. (canceled)

35. The method of any one of claims 1-16, 18, 22-23, 25, 28-30, and 32, wherein the fatty acid derivative is produced at a concentration of about 1 g/L or more.

36. (canceled)

37. (canceled)

38. (canceled)

39. The method of any one of claims 1-16, 18, 22-23, 25, 28-30, and 32, wherein the fatty acid derivative is produced at a concentration of about 1 g/L to about 200 g/L.

40. (canceled)

41. The method of claim 15, wherein the fatty ester is a fatty acid methyl ester.

42. The method of claim 41, wherein the fatty acid methyl ester is produced at a concentration of about 1 g/L or more.

43. (canceled)

44. (canceled)

45. (canceled)

46. The method of claim 41, wherein the fatty acid methyl ester is produced at a concentration of about 1 g/L to about 200 g/L.

47. (canceled)

48. The method of any one of claims 1-47, wherein the host cell is selected from the group consisting of a mammalian cell, plant cell, insect cell, yeast cell, fungus cell, filamentous fungi cell, cyanobacterial cell, and bacterial cell.

49. The method of claim 48, wherein the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

50. The method of claim 48, wherein the host cell is a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, a Mucor michei cell, a Streptomyces lividans cell or a Streptomyces murinus cell, an Actinomycetes cell, a Saccharomyces cerevisiae cell, a CHO cell, a COS cell, a VERO cell, a BHK cell, a HeLa cell, a Cv1 cell, an MDCK cell, a 293 cell, a 3T3 cell, or a PC12 cell.

51. The method of claim 48, wherein the host cell is an E. coli cell.

52. The method of claim 51, wherein the E. coli cell is a strain B, a strain C, a strain K, or a strain W E. coli cell.

53. The method of claim 48, wherein the host cell is a Synechococcus sp. PCC7002 cell, Synechococcus elongatus PCC7942 cell, or a Synechocystis sp. PCC6803 cell.

54. A fatty acid derivative produced by the method of any one of claims 1-16, 18, 22-23, 25, 28-30, 32, 35, 39, 41-42, 46, and 48-53.

55. A fatty ester produced by the method of any one of claims 1-16, 18, 22-23, 25, 28-30, 32, 35, 39, 41-42, 46, and 48-53.

56. The fatty ester of claim 55, wherein the fatty ester comprises an A side and a B side.

57. The fatty ester of claim 56, wherein the B side of the fatty ester is at least about 4 carbons in length.

58. (canceled)

59. (canceled)

60. (canceled)

61. (canceled)

62. (canceled)

63. (canceled)

64. (canceled)

65. The fatty ester of claim 55, selected from methyl dodecanoate, methyl 5-dodecenoate, methyl tetradecanoate, methyl 7-tetradecenoate, methyl hexadecanoate, methyl 9-hexadecenoate, methyl octadecanoate, methyl 11-octadecenoate, or combinations thereof.

66. The method of claim 15, wherein the fatty ester is a fatty acid ethyl ester.

67. The method of claim 28, wherein the fatty acid derivative is produced at a yield of about 0.5 g per 100 g of glucose or more in the culture medium.

68. (canceled)

69. (canceled)

70. (canceled)

71. The method of claim 28, wherein the fatty acid derivative is produced at a yield of about 0.5 g to about 30 g per 100 g of glucose in the culture medium.

72. (canceled)

73. The method of claim 1, wherein the fatty acid derivative is produced at a yield of about 10% or more by mass of carbon in the carbon source.

74. (canceled)

75. (canceled)

76. The method of claim 1, wherein the fatty acid derivative is produced at a yield of about 10% to about 95% by mass of carbon in the carbon source.

77. (canceled)

78. The method of claim 28, wherein the fatty acid derivative is produced at a yield of about 0.5% or more by mass of glucose in the culture medium.

79. (canceled)

80. The method of claim 28, wherein the fatty acid derivative is produced at a yield of about 0.5% to about 50% by mass of glucose in the culture medium.

81. (canceled)

82. A genetically engineered microorganism comprising at least one of a gene encoding a thioesterase, a gene encoding an acyl-CoA synthase, and a gene encoding an ester synthase, wherein the microorganism produces an increased level of a fatty ester relative to a wild-type microorganism.

83. A genetically engineered microorganism comprising an exogenous control sequence stably incorporated into the genomic DNA of the microorganism upstream of one or more of at least one of a gene encoding a thioesterase, a gene encoding an acyl-CoA synthase, and a gene encoding an ester synthase, wherein the microorganism produces an increased level of a fatty ester relative to a wild-type microorganism.

84. The genetically engineered microorganism of claim 83, wherein the exogenous control sequence is a promoter.

85. The genetically engineered microorganism of claim 84, wherein the promoter is a developmentally-regulated, organelle-specific, tissue-specific, inducible, constitutive, or cell-specific promoter.

86. The genetically engineered microorganism of claim 82 or 83, wherein the microorganism is genetically engineered to express, relative to a wild type microorganism, a decreased level of at least one of a gene encoding an acyl-CoA dehydrogenase, a gene encoding an outer membrane protein receptor, and a gene encoding a transcriptional regulator of fatty acid biosynthesis.

87. The genetically engineered microorganism of claim 82 or 83, wherein the microorganism is genetically engineered such that at least one of a gene encoding an acyl-CoA dehydrogenase, a gene encoding an outer membrane protein receptor, and a gene encoding a transcriptional regulator of fatty acid biosynthesis is deleted.

88. The genetically engineered microorganism of claim 86 or 87, wherein the gene encoding an acyl-CoA dehydrogenase is fadE.

89. The genetically engineered microorganism of claim 86 or 87, wherein the gene encoding the outer membrane protein receptor encodes a ferrichrome outer membrane transporter.

90. The genetically engineered microorganism of claim 89, wherein the gene encoding the ferrichrome outer membrane transporter is fhuA.

91. The genetically engineered microorganism of claim 86 or 87, wherein the gene encoding the transcriptional regulator encodes a DNA binding transcriptional repressor.

92. The genetically engineered microorganism of claim 91, wherein the gene encoding the DNA binding transcriptional repressor is fabR.

93. The genetically engineered microorganism of any one of claims 82-92, selected from a Gram-negative or a Gram-positive bacterium.

94. The genetically engineered microorganism of claim 93, selected from an E. coli, mycrobacterium, Nocardia sp., Nocardia farcinica, Streptomyces griseus, Salinispora arenicola, Clavibacter michiganenesis, Acinetobacter, Alcanivorax, Alcaligenes, Arabidopsis, Fundibacter, Marinobacter, Mus musculus, Pseudomonas, or Simmodsia, Yarrowia, Candida, Rhodotorula, Rhodosporidium, Cryptococcus, Trichosporon, or Lipomyces.

95. The genetically engineered microorganism of any one of claim 94, selected from an E. coli strain B, strain C, strain K, or strain W.

96. The genetically engineered microorganism of any one of claims 82-92, selected from a Synechococcus sp. PCC7002, Synechoccus elongatus PCC7942, or Synechocystis sp. PCC6803.

97. A method of producing a fatty acid derivative comprising culturing the genetically engineered microorganism of any one of claims 82-96, in the presence of an alcohol.

98. A fatty acid derivative produced by the method of claim 97.

99. The fatty acid derivative of claim 98, wherein the fatty acid derivative is a fatty ester.

100. A biofuel composition comprising the fatty acid derivative of any one of claims 54-57, 65, and 98-99.


SEQUENCE LISTING (34 sequences)

SEQ ID NO: 1 - DNA, 70 bp, Artificial Sequence
  Synthetic primer
aaaaacagcaacaatgtgagctttgttgtaattatattgtaaacatattgattccggggatccgtcgacc

SEQ ID NO: 2 - DNA, 68 bp, Artificial Sequence
  Synthetic primer
aaacggagcctttcggctccgttattcatttacgcggcttcaactttcctgtaggctggagctgcttc

SEQ ID NO: 3 - DNA, 23 bp, Artificial Sequence
  Synthetic primer
cgggcaggtgctatgaccaggac

SEQ ID NO: 4 - DNA, 23 bp, Artificial Sequence
  Synthetic primer
cgcggcgttgaccggcagcctgg

SEQ ID NO: 5 - DNA, 70 bp, Artificial Sequence
  Synthetic primer
atcattctcgtttacgttatcattcactttacatcagagatataccaatgattccggggatccgtcgacc

SEQ ID NO: 6 - DNA, 69 bp, Artificial Sequence
  Synthetic primer
gcacggaaatccgtgccccaaaagagaaattagaaacggaaggttgcggttgtaggctggagctgcttc

SEQ ID NO: 7 - DNA, 21 bp, Artificial Sequence
  Synthetic primer
caacagcaacctgctcagcaa

SEQ ID NO: 8 - DNA, 21 bp, Artificial Sequence
  Synthetic primer
aagctggagcagcaaagcgtt

SEQ ID NO: 9 - DNA, 6700 bp, Artificial Sequence
  pHZ1.61 plasmid
ggggaattgtgagcggataacaattcccctgtagaaataattttgtttaactttaataaggagatataccatggtgaagaaggtttggcttaaccgttatcccgcggacgttccgacggagatcaaccctgaccgttatcaatctctggtagatatgtttgagcagtcggtcgcgcgctacgccgatcaacctgcgtttgtgaatatgggggaggtaatgaccttccgcaagctggaagaacgcagtcgcgcgtttgccgcttatttgcaacaagggttggggctgaagaaaggcgatcgcgttgcgttgatgatgcctaatttattgcaatatccggtggcgctgtttggcattttgcgtgccgggatgatcgtcgtaaacgttaacccgttgtataccccgcgtgagcttgagcatcagcttaacgatagcggcgcatcggcgattgttatcgtgtctaactttgctcacacactggaaaaagtggttgataaaaccgccgttcagcacgtaattctgacccgtatgggcgatcagctatctacggcaaaaggcacggtagtcaatttcgttgttaaatacatcaagcgtttggtgccgaaataccatctgccagatgccatttcatttcgtagcgcactgcataacggctaccggatgcagtacgtcaaacccgaactggtgccggaagatttagcttttctgcaatacaccggcggcaccactggtgtggcgaaaggcgcgatgctgactcaccgcaatatgctggcgaacctggaacaggttaacgcgacctatggtccgctgttgcatccgggcaaagagctggtggtgacggcgctgccgctgtatcacatttttgccctgaccattaactgcctgctgtttatcgaactgggtgggcagaacctgcttatcactaacccgcgcgatattccagggttggtaaaagagttagcgaaatatccgtttaccgctatcacgggcgttaacaccttgttcaatgcgttgctgaacaataaagagttccagcagctggatttctccagtctgcatctttccgcaggcggagggatgccagtgcagcaagtggtggcagagcgttgggtgaaactgacaggacagtatctgctggaaggctatggccttaccgagtgtgcgccgctggtcagcgttaacccatatgatattgattatcatagtggtagcatcggtttgccggtgccgtcgacggaagccaaactggtggatgatgatgataatgaagtaccaccgggtcaaccgggtgagctttgtgtcaaaggaccgcaggtgatgctgggttactggcagcgtccggatgctacagatgagatcatcaaaaatggctggttacacaccggcgacatcgcggtgatggatgaagaagggttcctgcgcattgtcgatcgtaaaaaagacatgattctggtttccggttttaacgtctatcccaacgagattgaagatgtcgtcatgcagcatcctggcgtacaggaagtcgcggctgttggcgtaccttccggctccagtggtgaagcggtgaaaatcttcgtagtgaaaaaagatccatcgcttaccgaagagtcactggtgaccttttgccgccgtcagctcacgggctacaaagtaccgaagctggtggagtttcgtgatgagttaccgaaatctaacgtcggaaaaattttgcgacgagaattacgtgacgaagcgcgcggcaaagtggacaataaagcctgaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcgcccattacatccgattgattttatattcctgtcactagaaaaaagacaacagcctatgcatgtaggtggtttatttttgtttcagattcctgataacgccccagacacctttattcaagatctggtgaatgatatccggatatcaaaatcaatccctgttccaccattcaacaataaactgaatgggcttttttgggatgaagatgaagagtttgatttagatcatcattttcgtcatattgcactgcctcatcctggtcgtattcgtgaattgcttatttatatttcacaagagcacagtacgctgctagatcgggcaaagcccttgtggacctgcaatattattgaaggaattgaaggcaatcgttttgccatgtacttcaaaattcaccatgcgatggtcgatggcgttgctggtatgcggttaattgaaaaatcactctcccatgatgtaacagaaaaaagtatcgtgccaccttggtgtgttgagggaaaacgtgcaaagcgcttaagagaacctaaaacaggtaaaattaagaaaatcatgtctggtattaagagtcagcttcaggcgacacccacagtcattcaagagctttctcagacagtatttaaagatattggacgtaatcctgatcatgtttcaagctttcaggcgccttgttctattttgaatcagcgtgtgagctcatcgcgacgttttgcagcacagtcttttgacctagatcgttttcgtaatattgccaaatcgttgaatgtgaccattaatgatgttgtactagcggtatgttctggtgcattacgtgcgtatttgatgagtcataatagtttgccttcaaaaccattaattgccatggttccagcctctattcgcaatgacgattcagatgtcagcaaccgtattacgatgattctggcaaatttggcaacccacaaagatgatcctttacaacgtcttgaaattatccgccgtagtgttcaaaactcaaagcaacgcttcaaacgtatgaccagcgatcagattctaaattatagtgctgtcgtatatggccctgcaggactcaacataatttctggcatgatgccaaaacgccaagccttcaatctggttatttccaatgtgcctggcccaagagagccactttactggaatggtgccaaacttgatgcactctacccagcttcaattgtattagacggtcaagcattgaatattacaatgaccagttatttagataaacttgaagttggtttgattgcatgccgtaatgcattgccaagaatgcagaatttactgacacatttagaagaagaaattcaactatttgaaggcgtaattgcaaagcaggaagatattaaaacagccaattaaaaacaataaacttgattttttaatttatcagataaaactaaagggctaaattagccctcctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 10 - DNA, 4963 bp, Artificial Sequence
  pHZ1.97-atfA1 plasmid
ggggaattgtgagcggataacaattcccctgtagaaataattttgtttaactttaataaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaaagcgcttagcccagtggatcaactgttcctgtggctggaaaaacgacagcaacccatgcacgtaggcggtttgcagctgttttccttcccggaaggtgccggccccaagtatgtgagtgagctggcccagcaaatgcgggattactgccacccagtggcgccattcaaccagcgcctgacccgtcgactcggccagtattactggactagagacaaacagttcgatatcgaccaccacttccgccacgaagcactccccaaacccggtcgcattcgcgaactgctttctttggtctccgccgaacattccaacctgctggaccgggagcgccccatgtgggaagcccatttgatcgaagggatccgcggtcgccagttcgctctctattataagatccaccattcggtgatggatggcatatccgccatgcgtatcgcctccaaaacgctttccactgaccccagtgaacgtgaaatggctccggcttgggcgttcaacaccaaaaaacgctcccgctcactgcccagcaacccggttgacatggcctccagcatggcgcgcctaaccgcgagcataagcaaacaagctgccacagtgcccggtctcgcgcgggaggtttacaaagtcacccaaaaagccaaaaaagatgaaaactatgtgtctatttttcaggctcccgacacgattctgaataataccatcaccggttcacgccgctttgccgcccagagctttccattaccgcgcctgaaagttatcgccaaggcctataactgcaccattaacaccgtggtgctctccatgtgtggccacgctctgcgcgaatacttgattagccaacacgcgctgcccgatgagccactgattgcaatggtgcccatgagcctgcggcaggacgacagcactggcggcaaccagatcggtatgatcttggctaacctgggcacccacatctgtgatccagctaatcgcctgcgcgtcatccacgattccgtcgaggaagccaaatcccgcttctcgcagatgagcccggaagaaattctcaatttcaccgccctcactatggctcccaccggcttgaacttactgaccggcctagcgccaaaatggcgggccttcaacgtggtgatttccaacatacccgggccgaaagagccgctgtactggaatggtgcacagctgcaaggagtgtatccagtatccattgccttggatcgcatcgccctaaatatcaccctcaccagttatgtagaccagatggaatttgggcttatcgcctgccgccgtactctgccttccatgcagcgactactggattacctggaacagtccatccgcgaattggaaatcggtgcaggaattaaatagtaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 11 - DNA, 5733 bp, Artificial Sequence
  pACYC-PTrc vector
actcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgatacgcctatttttataggttaatgtcatgataataatggtttcttagacgtcttaattaatcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctggagaccgtttaaactcaatgatgatgatgatgatggtcgacggcgctattcagatcctcttctgagatgagtttttgttcgggcccaagcttcgaattcccatatggtaccagctgcagatctcgagctcggatccatggtttattcctccttatttaatcgatacattaatatatacctctttaatttttaataataaagttaatcgataattccggtcgagtgcccacacagattgtctgataaattgttaaagagcagtgccgcttcgctttttctcagcggcgctgtttcctgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgatgataagctgtcaaaccagatcaattcgcgctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcggggccggcctcg

SEQ ID NO: 12 - DNA, 60 bp, Artificial Sequence
  Synthetic primer
ctctagaaataatttaactttaagtaggagauaggtacccatggcggacacgttattgat

SEQ ID NO: 13 - DNA, 58 bp, Artificial Sequence
  Synthetic primer
cttcgaattccatttaaattatttctagagtcattatgagtcatgatttactaaaggc

SEQ ID NO: 14 - DNA, 65 bp, Artificial Sequence
  Synthetic primer
ctctagaaataattttagttaagtataagaaggagatataccatggtgaagaaggtttggcttaa

SEQ ID NO: 15 - DNA, 51 bp, Artificial Sequence
  Synthetic primer
cttcgaattccatttaaattatttctagagttatcaggctttattgtccac

SEQ ID NO: 16 - DNA, 42 bp, Artificial Sequence
  Synthetic primer
ctctagaaataatttagttaagtataagaaggagatatacat

SEQ ID NO: 17 - DNA, 58 bp, Artificial Sequence
  Synthetic primer
cttcgaattccatttaaattatttctagagttactatttaattcctgcaccgatttcc

SEQ ID NO: 18 - DNA, 28 bp, Artificial Sequence
  Synthetic primer
atatgacgtcggcatccgcttacagaca

SEQ ID NO: 19 - DNA, 32 bp, Artificial Sequence
  Synthetic primer
aattcttaagtcaggagagcgttcaccgacaa

SEQ ID NO: 20 - DNA, 36 bp, Artificial Sequence
  Synthetic primer
caaccagcggccgcgcagacgatggtgcaggatatc

SEQ ID NO: 21 - DNA, 36 bp, Artificial Sequence
  Synthetic primer
ccacacactagtcagatctgcagaattcaggctgtc

SEQ ID NO: 22 - DNA, 22 bp, Artificial Sequence
  Synthetic primer
ggctggctggcataaatatctc

SEQ ID NO: 23 - DNA, 19 bp, Artificial Sequence
  Synthetic primer
catcgcgtgggcgtattcg

SEQ ID NO: 24 - DNA, 71 bp, Artificial Sequence
  Synthetic primer
cgtacctctatcttgatttgcttgtttcattactcgtccttcacatttccgtgtaggctggagctgcttcg

SEQ ID NO: 25 - DNA, 70 bp, Artificial Sequence
  Synthetic primer
atgttttattgcgttaccgttcattcacaatactggagcaatccagtatgattccggggatccgtcgacc

SEQ ID NO: 26 - DNA, 20 bp, Artificial Sequence
  Synthetic primer
aaccggccaaagaattgcag

SEQ ID NO: 27 - DNA, 20 bp, Artificial Sequence
  Synthetic primer
taagccagcaactaacgcca

SEQ ID NO: 28 - DNA, 4311 bp, Artificial Sequence
  pLacZ plasmid
ctagtaacggccgccagtgtgctggaattcaggcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagcctgaattctgcagatctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctccc

SEQ ID NO: 29 - DNA, 25 bp, Artificial Sequence
  Synthetic primer
atgtctgacaaggggtttgacccct

SEQ ID NO: 30 - DNA, 25 bp, Artificial Sequence
  Synthetic primer
gcacatccttatccaattgctctag

SEQ ID NO: 31 - DNA, 25 bp, Artificial Sequence
  Synthetic primer
gatcaaacaggtgcagcagcaactt

SEQ ID NO: 32 - DNA, 25 bp, Artificial Sequence
  Synthetic primer
attcttgacaagcgatcgcggtcac

SEQ ID NO: 33 - DNA, 25 bp, Artificial Sequence
  Synthetic primer
attcaatacacccccctagccgatc

SEQ ID NO: 34 - DNA, 25 bp, Artificial Sequence
  Synthetic primer
taagggtggtgggaaaaatgggcca